(19) United States
(12) Patent Application Publication
(10) Pub. No.: US 2026/0306421 A1
(43) Pub. Date: Oct. 8, 2026

(54) SYSTEMS AND METHODS FOR TRANSDERMAN PATCH DELIVERY

(71) Applicant: VITAMAX PATCH WHOLESALER LLC, Jupiter, FL (US)

(72) Inventors: Stuart JONES, Hertfordshire (GB); Mohamed ALHNAN, Jupiter, FL (US)

(21) Appl. No.: 19/100,463

(22) PCT Filed: Jul. 31, 2023
(86) PCT No.: PCT/US2023/071320

§ 371 Date: Jan. 31, 2025

Related U.S. Application Data
(60) Provisional application No. 63/394,219, filed on Aug. 1, 2022.

(51) Int. Cl. A61K 9/70; A61K 31/295; A61K 31/30; A61K 31/315; A61K 31/593

(52) U.S. Cl. CPC A61K 9/7061; A61K 31/295; A61K 31/30; A61K 31/315; A61K 31/593

(57) ABSTRACT

A patch for transdermal delivery of a compound, including: a backing layer; an active agent layer comprising at least one compound; and a permeable-membrane release layer configured to transfer the at least one compound to a skin surface.

### CROSS-REFERENCE TO RELATED APPLICATIONS

The present application claims priority to U.S. Provisional Patent Application No. 63/394,219, filed Aug. 1, 2022, the entire contents of which are hereby incorporated by reference.

### STATEMENT REGARDING FEDERALLY FUNDED RESEARCH

Not applicable.

### BACKGROUND

Zinc, iron, and vitamin D are essential nutrients that are actively absorbed and transported in the body. Despite the availability of zinc, iron, and vitamin D in several foods, inadequate intake, malabsorption, lifestyle and environmental factors which reduce exposure to sunlight, or increased requirements for zinc, iron, and Vitamin D can all cause deficiencies in one or more of these essential nutrients.

Zinc, iron, and vitamin D supplements are available commercially alone or in combination with other minerals in the form of oral tablets or capsules. There are a number of barriers to efficient oral uptake of zinc, iron, and vitamin D, however. For example, zinc readily forms coordination complexes with hydroxide ions in the small intestines. These complexes can hinder the metal's delivery and its biological activity. Existing vitamin D supplements have limited absorption of vitamin D via oral administration. Absorption of iron by oral supplementation is not efficient (typically between 10-15%) and often cause gastric side-effects and this renders them unpopular with many subjects in need.

Transdermal administration of therapeutically beneficial amounts of zinc, iron, and vitamin D is highly attractive in terms of tolerability, patient compliance, and ease of administration. However, studies have reported that the skin is a significant barrier to charged molecules and, as a result, delivering a therapeutically beneficial amount of a charged nutrients through the skin is not trivial. For example, existing vitamin D transdermal patches fail to readily release vitamin D and, in combination with low rates of skin permeation, require the subject in need to wear the patches for long periods of time. Common coordination complexes of zinc do not readily cross human skin. Previous work on transdermal delivery of iron shows that the both the stratum corneum and the epidermis/dermis present a barrier to the delivery of iron and that optimization of iron compounds for transdermal delivery are necessary.

Therefore, a need exists for patches, methods of using, and methods of synthesizing nutrients capable of efficient transdermal delivery of therapeutic amounts of nutrients, such as zinc, iron, and vitamin D, to a subject in need.

### BRIEF SUMMARY OF THE INVENTION

In one embodiment, a patch for transdermal delivery of a compound, including: a backing layer; an active agent layer including at least one compound; and a permeable-membrane release layer configured to transfer the at least one compound to a skin surface.

In another embodiment, a method of delivering a compound to a subject in need, including: applying a patch for transdermal delivery of a compound, including: an outer backing layer; an intermediate active agent layer including at least one compound; and a permeable-membrane release layer configured to transfer the at least one compound to a skin surface.

In still another embodiment, a method of synthesizing a nutrient-counter ion salt for transdermal delivery, including: providing at least one nutrient; providing at least one counter ion; solubilizing the at least one nutrient and the at least one counter ion in at least one solvent to produce a solution; optionally adjusting a pH of the solution; and removing the solvent from the solution to produce a nutrient-counter ion salt.

In yet another embodiment, a patch for transdermal delivery of a compound, including: an outer backing layer including polyester; an intermediate active agent layer including a supersaturated solution of zinc ethyl maltol and PEG400:PEG 1000 (8% w/w:2% w/w); and a permeable-membrane release layer configured to transfer the at least one compound to a skin surface, wherein the permeable-membrane release layer is nylon.

In still another embodiment, a patch for transdermal delivery of a compound, including: an outer backing layer including polyester; an intermediate active agent layer including a solution of 18 mg/mL iron maltol in polyethylene glycol; and a permeable-membrane release layer configured to transfer the at least one compound to a skin surface, wherein the permeable membrane release layer is nylon.

In yet another embodiment, a patch for transdermal delivery of a compound, including: an outer backing layer including polyester; an intermediate active agent layer including: a compound including cholecalciferol phosphate sodium; a penetration enhancer including polyethylene glycol; and an adhesive including acrylates copolymer; and a permeable-membrane release layer configured to transfer the at least one compound to a skin surface, wherein the permeable-membrane release layer is ethylene vinyl acetate.

### BRIEF DESCRIPTIONS OF THE DRAWINGS

FIG. 1A is a top view of a non-limiting example transdermal patch that may be used in accordance with the present disclosure.

FIG. 1B is an exploded side perspective view of the transdermal patch of FIG. 1A.

FIG. 2 is a flowchart of non-limiting example steps of a method for optimization of a transdermal patch.

FIG. 3 depicts structures for non-limiting example zinc complexes that may be used in accordance with the present invention.

FIG. 4 depicts a schematic representation of a Franz cell.

FIG. 5 shows the effect of different post-column reagent (PAR) flow rates on zinc acetate samples' absorbance in HPLC. The number of injections was 3 for each flow rate.

FIG. 6 shows recovery of zinc chloride and zinc ethyl maltol from porcine skin. Recovery percentage calculated using A) zinc acetate calibration curve in water, B) zinc chloride and zinc ethyl maltol in PG/PDCA. Data represents the mean±one standard deviation (n=3).

FIG. 7 shows Zinc Chloride and Zinc Ethyl Maltol in PG, Skin deposition and transdermal permeation. Data represents the mean±one standard deviation (n=5).

FIG. 8 shows Zinc Chloride in PG and Zinc Ethyl Maltol in PEG400, Skin deposition and transdermal permeation. Data represents the mean±one standard deviation (n=5).

FIG. 9 shows skin permeation and deposition studies of Zinc Ethyl Maltol in PEG400 and PEG400:PEG1000 (8% w/w:2% w/w). Data represents the mean±one standard deviation (n=5).

FIG. 10 depicts a schematic of the work-flow for the development of drug in adhesive patch.

FIG. 11 shows a photograph of (A) inner side and (B) outer side of reservoir based on supersaturated solution of zinc ethyl maltol in PEG400:PEG1000 (60 mg/mL).

FIG. 12 shows transdermal permeation and deposition of elemental zinc from Zinc Ethyl Maltol reservoir patch. Data represents mean±one standard deviation (n=5).

FIG. 13 shows a schematic representation of in vitro and ex vivo permeation studies using Franz cells.

FIG. 14 depicts structures for non-limiting example iron complexes that may be used in accordance with the present invention.

FIG. 15 shows skin necrosis post iron chloride hexahydrate permeation.

FIG. 16 shows iron chloride hexahydrate skin permeation and deposition after 24 h.

FIG. 17 shows iron ethyl maltol skin permeation and deposition after 24 h.

FIG. 18 shows iron oleate permeation and deposition after 24 h.

FIG. 19 shows permeation and deposition of iron (ferric) propionate and iron maltol solutions.

FIG. 20 shows photographs of skin after the 24 h permeation study, a) iron propionate, b) iron maltol (made at pH 4) in 7.5:1.5:1:1 water:PG:dimethicone:ethanol, c) iron maltol (made at pH 4) in PG, and d) iron maltol (made at pH 1.6) in Transcutol.

FIG. 21 shows workflow for the development of drug in adhesive patch.

FIG. 22 a) 4% loading (2 mg FeEM, 45 mg 9301 and 2 mg PG), b) 7.5% loading (4 mg FeEM, 45 mg 9301 and 4 mg PG), and c) 10% loading (5.5 mg FeEM, 45 mg Duro-TAK 9301 and 5.5 mg PG).

FIG. 23 shows an inner face of a 34 mm diameter patch based on iron ethyl maltol in acrylate adhesive.

FIG. 24 shows adhesive patch deposition of iron using iron (ferric) complexes.

FIG. 25 shows an example of reservoir patch R3 including 11 mg/mL iron maltol in PG using a Durapore membrane).

FIG. 26 shows reservoir patches permeation and deposition. R1 FeProp is iron proprionate dissolved in propylene glycol, R2 included 18 mg/mL iron maltol complex in PG using a nylon rate controlling membrane, R3 included 11 mg/mL iron maltol in PG using a Durapore membrane and R4 included 16 mg/mL iron maltol in 7.5:1.5:1:1 water:PG:dimethicone using a Durapore membrane.

FIG. 27 shows a photograph of a patch (left) and skin (right) post 24 hours permeation study; a) Patch R2, b) Patch R3, and c) Patch R4.

FIG. 28 shows a summary of formulations showing the best solution (iron maltol made at pH 4 in PG), formulating it into a nylon membrane reservoir patch, the best iron maltol adhesive patch and an existing commercial iron transdermal patch.

FIG. 29 shows chemical synthesis of cholecalciferol phosphate disodium from cholecalciferol.

FIG. 30 shows stability of cholecalciferol or cholecalciferol phosphate disodium in different solvents over 24 hours. Data represents the mean±one standard deviation (n=3).

FIG. 31 shows cholecalciferol and cholecalciferol phosphate skin permeation and deposition. Data represents the mean±one standard deviation (n=3).

FIG. 32 shows an example of optimizing vitamin D phosphate concentration in Oppanol-based patch (F2).

FIG. 33 shows the release of cholecalciferol phosphate disodium through cellulose membrane using different combination of solvents for donor and receiver solvents. n=5. Data is a mean±one standard deviation.

FIG. 34 shows cholecalciferol phosphate release from DURO-TAK 387-2516 patch (patch) compared to release from a propylene glycol solution (PG sol) n=5. Data is a mean±one standard deviation.

FIG. 35 shows the release of six different cholecalciferol phosphate acid patches over 24 h. Data is mean±one standard deviation (n=5).

FIG. 36 shows stability of cholecalciferol phosphate acid over 24 hours in the donor, extraction and receiver solvents.

FIG. 37 shows cholecalciferol phosphate acid patch permeation. Data represents mean±one standard deviation (n=5).

### DETAILED DESCRIPTION OF THE INVENTION

Disclosed herein are patches and compositions for transdermal delivery of a compound, methods of using the same, and methods of synthesizing a nutrient-counter ion salt for transdermal delivery. As used herein, the term ‘transdermal delivery’ refers to the delivery of at least one compound through skin (i.e., epidermis, dermis, hypodermis) with the intent for the compound to be pass through skin layers and exert a systemic effect on a subject in need. Transdermal delivery may involve transferring (i.e., exposing, bringing into contact with, adsorbing into, permeating, or otherwise making available for systemic effect) a compound to a skin surface.

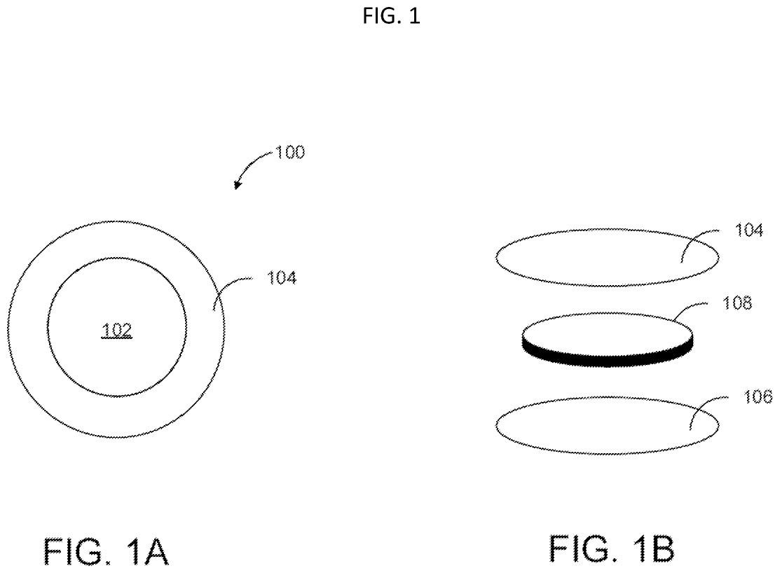

Disclosed herein is a patch for transdermal delivery of a compound, including a backing layer, an active agent layer including at least one compound, and a permeable-membrane release layer configured to transfer the at least one compound to a skin surface. Referring to FIGS. 1A and 1, a non-limiting example of a transdermal patch 100 is shown in a top view (FIG. 1A) and an exploded side perspective (FIG. 1). The transdermal patch may include a backing layer 104. In some instances, the backing layer 104 may be configured to provide structural support for the transdermal patch 100, to protect the contents of an active agent layer 102, to provide for an aesthetic surface when the transdermal patch 100 is placed on the skin of a subject. In some configurations, the backing layer 104 may be a carrier foil layer. In some cases, the backing layer may include films of polyesters, polyolefins, polyurethanes, or any combinations thereof. Suitable commercially available backing layer may include 3M™ Scotchpak™ Polyester Backing Film Laminate 9730.

An active agent layer 102 may be formed between backing layer 104 and a permeable-membrane release layer 106 and may contain at least one compound 108. In some cases, the permeable-membrane release layer 106 may be a release foil or may act as a protecting layer between the skin of a subject and the transdermal patch 100. The permeable-membrane release layer may be permeable or may include openings to allow transfer between the at least one compound 108 and the skin of a user. In some cases, the permeable-membrane release layer may include nylon, regenerated cellulose, hydrophobic polyvinylidene fluoride (PVDF), ethylene vinyl acetate, or fluoropolymer on polyester film. Suitable commercially available permeable-membrane release layer may include regenerated cellulose (12-14 k molecular weight cut-off Medical International), Whatman nylon (0.20 μm), and 3M™ CoTran 9728.

The patches disclosed herein include an active agent layer (102) including at least one compound (108) (i.e., a composition for transdermal delivery). The at least one compound may include zinc, iron, vitamin D (i.e., vitamin D1 as a mixture of ergocalciferol with lumisterol, vitamin D2 as ergocalciferol, vitamin D3 as cholecalciferol, vitamin D4 as 22-dihydroergocalciferol, vitamin D5 as sitocalciferol, and any derivatives thereof, including cholecalciferol phosphate), copper, selenium, magnesium, or any combinations thereof. In some instances, the at least one compound is a salt including a nutrient and at least one counter ion. For example, the nutrient may be Zn⁺, Zn²⁺, Fe²⁺, Fe³⁺, Fe⁴⁺, Fe⁶⁺, cholecalciferol PO₄²⁻, or any combinations thereof. Counter ions may be selected based upon their ability to neutralize the charge of the nutrient. In some embodiments the counter ion may include ethyl maltol, maltol, kojic acid, propionate, hexanoate, oleate, chloride, phosphate, or any combination thereof. In some cases, the at least one compound may be a nutrient-counter ion salt including iron ethyl maltol, iron maltol, iron kojic acid, iron propionate, iron hexanoate, iron oleate, zinc ethyl maltol, zinc maltol, zinc kojic acid, zinc propionate, or any combinations thereof.

A non-limiting example patch or composition configured for vitamin D delivery may include vitamin D that has been phosphorylated to provide for permeation through the skin. The synthesis and resulting compound may be optimized for skin delivery.

It may be desirable to have a combination of compounds or compositions present in the active agent layer of the same patch. By way of example, an active agent layer may contain zinc and iron, zinc and vitamin D, iron and vitamin D, or zinc and iron and vitamin D. It may be desirable to include combinations of compounds or compositions which are synergistically absorbed or have a particular health benefit when administered at the same time, such as a patch which contains a combination of vitamin D and calcium.

The patches disclosed herein include an active agent layer (102) including at least one compound (108). In some embodiments, the active agent layer includes at least one compound or composition, and further includes a solvent. These embodiments may also be referred to as ‘reservoir patches.’ The solvent may be selected and optimized for solubilizing the at least one compound or composition. In some cases, it may be desirable to fully solubilize the at least one compound or composition. In other cases, it may be desirable to partially solubilize the at least one compound or composition (i.e., some solids remain undissolved). It may be desirable for the active agent layer to include a supersaturated solution of the at least one compound or composition in a solution. A supersaturated solution is a solution which contains more than the maximum concentration of solute (i.e., the at least one compound or composition) than is capable of being dissolved in a given volume of solvent at a given temperature. Suitable solvents may include propylene glycol, methanol, ethyl acetate, ethanol, hexane, polyethylene glycol, diethylene glycol monoethyl ether, glycerol, triacetin, miglyol, liquid paraffin, sodium laureth sulfate, Tween 85, Span 20, Kolliphore®, Natrsol M250, dimethicone, or any combination thereof.

A non-limiting example patch configured as a zinc ethyl maltol reservoir patch may be made from a composition of 60 mg of zinc ethyl maltol dissolved in 3 mL of methanol, followed by adding 1 mL of PEG 400:PEG1000 (8% w/w:2% w/w). Significant transdermal permeation may be provided by a supersaturated solution of PEG400:PEG1000. A supersaturated solution based on PEG400/PG6000 may be used to achieve 28.8 mg/mL of elemental zinc.

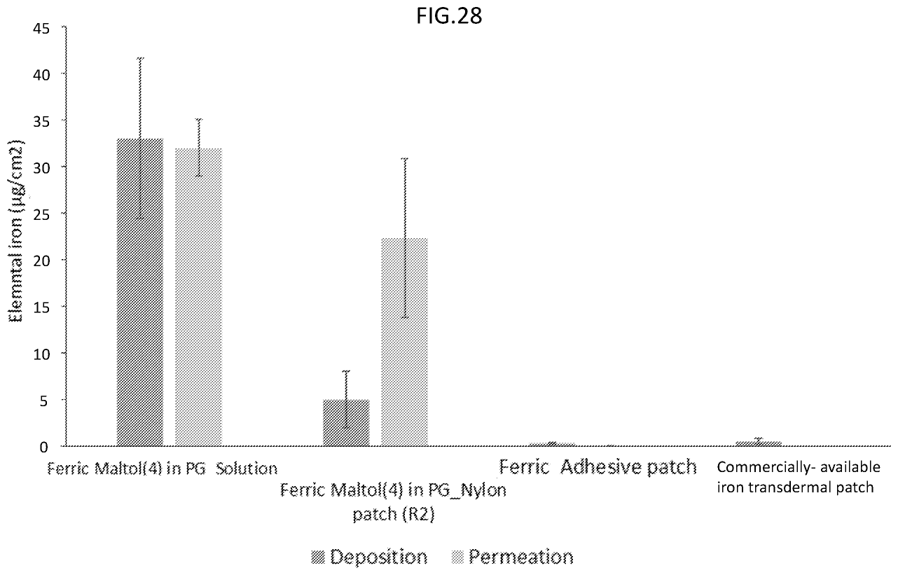

A non-limiting example patch configured as an iron delivery patch or composition may include a range of suitable excipients and solvents to formulate a solution of high concentration that could be loaded into the patch reservoir. Potential solvents include: ethanol, methanol, PG, PEG400, SDS, Tween 85, Span 20, Kolliphore®, Natrsol M250 and dimethicone. The reservoir patch or composition may be made via heat sealing a packing material onto a controlled release membrane. To achieve this, the patch backing (3M™ Scotchpak™ Polyester Backing Film Laminate 9730, UK) may be placed on the surface of a heat press machine (HP230B, China), a 200 μL solution of the iron nutrient-counterion complex composition may be loaded onto the backing and then may be sealed using heat onto the controlled release membrane. Examples of iron reservoir patches and their performance as described herein are provided throughout the Examples and particularly in FIG. 28.

The patches disclosed herein include an active agent layer (102) including at least one compound (108) (i.e., a composition for transdermal delivery). In some embodiments, the active agent layer includes at least one compound or composition and further includes an adhesive. These embodiments may also be referred to as ‘drug-in-adhesive’ or ‘adhesive’ patches. In some embodiments, the at least one compound or composition is solubilized within the adhesive and is dried in the adhesive without crystallization (i.e., crystals, precipitates, particulates, solids). Suitable adhesives may be selected and optimized for solubilizing the at least one compound or composition. In some embodiments adhesives may include polyisobutene, acrylates copolymer, amine-compatible silicone, polymethacrylate, or any combination thereof.

The patches, methods, compounds, and compositions disclosed herein may be formulated to further include one or more polymers, penetration enhancers, stabilizers, plasticizers, pH regulators, thickeners, colorants, desiccants, solubility enhancers (i.e., solubilizer), or any combinations thereof. In some cases, the penetration enhancer may include polyethylene glycol. In some cases, the patch may further include one or more antioxidants. In some cases, the antioxidant may be vitamin E, vitamin E derivatives, butylated hydroxytoluene (BHT), butylated hydroxyanisole (BHA), L-cysteine, and propyl gallate. In some cases, the solubility enhancer may be Tween, such as Tween-85, or dodecyl amine.

The patches, methods, compounds, and compositions disclosed herein may also be further formulated to include one or more binding agents, filling agents, lubricating agents, suspending agents, flavoring agents, preservatives, buffers, wetting agents, disintegrants, buffers, bacteriostats, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

The transdermal patch 100 may be placed on a skin surface of a subject in need. As used herein, a “subject” may be interchangeable with “patient” or “individual” and means an animal, which may be a human or non-human animal. A “subject in need” may include a subject having a disease, disorder, or condition that is responsive to therapy with a patch for transdermal delivery of at least one compound, the patch including a backing layer, an active agent layer including at least one compound, and a permeable-membrane release layer configured to transfer the at least one compound to a skin surface. The at least one compound may include any one of or any combination of the presently disclosed compounds described above and below. For example, a “subject in need” may include a subject having inadequate nutritional intake, malabsorption, lifestyle and environmental factors which reduce exposure to sunlight, or increased requirements for the at least one compound. In some cases, the subject in need may have a disease associated with a nutrient deficiency. In other cases, the subject in need may have a disease which has at least one symptom, or at least one root cause, alleviated by transdermal delivery of at least one compound or composition. The subject in need may also include a subject for which there is a desire to increase levels of at least one compound (i.e., a composition for transdermal delivery), independent of whether the subject has a particular disease, disorder, or condition.

As used herein, the phrase “effective amount” or a “therapeutically beneficial amount” means that compound or composition dosage that provides the specific pharmacological response or amount delivered for which the compound or composition is administered in a significant number of subjects in need of such treatment. An effective amount of at least one compound or composition that is administered to a particular subject in a particular instance will not always be effective in treating the conditions/diseases described herein, even though such dosage is deemed to be a therapeutically effective amount by those of skill in the art.

The compounds and compositions utilized in the patches and methods disclosed herein may be formulated to include: (a) a therapeutically effective amount of at least one compound as disclosed herein; and (b) one or more pharmaceutically acceptable carriers, excipients, or diluents. The methods, patches, and compositions may be administered to provide the one or more compounds at a daily dose of about 0.05 mg, about 0.1 mg, about 0.2 mg, about 0.3 mg, about 0.5 mg, about 1.0 mg, about 1.5 mg, about 2.0 mg, about 2.5 mg, about 3.0 mg, about 3.5 mg, about 4.0 mg, about 4.5 mg, about 5.0 mg over 24 hours from approximately a 1 inch×1 inch or 3.1×3.1 cm patch.

The transdermal patch may position on the subject in need in any suitable placement, such as on an arm, leg, or trunk of the subject in need. Preparation of the area of skin where the patch will be placed may be prepared in order to optimize the adherence or contact of the patch to the skin. Skin preparations may include, cleansing, shaving, softening, dermaplaning (sometimes referred to as ‘dermatoming’), soaking, or any combinations thereof.

Disclosed herein is a method of delivering a compound or composition to a subject in need, including applying a patch for transdermal delivery of a compound, as described above and below. ‘Applying’ may include adhering, placing, laying, smoothing, or otherwise contacting the patch with the skin so that the permeable-membrane release layer, which permits the release of the at least one compound or composition out of the patch, lays in close proximity with an area of skin on the subject in need. It may be necessary for the skin of the subject in need to remain in contact with the patch for a duration of time in order for a therapeutically beneficial amount of the at least one compound or composition to transfer onto the skin. In some cases, the patch may remain in contact with the skin of the subject in need for about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, or about 24 hours.

Disclosed herein are compositions and methods of synthesizing a nutrient-counter ion salt for transdermal delivery including providing at least one nutrient, providing at least one counter ion, solubilizing the at least one nutrient and the at least one counter ion in at least one solvent to produce a solution, optionally adjusting a pH of the solution, and removing the solvent from the solution to produce a nutrient-counter ion salt. Suitable nutrients include those previously disclosed above and in Examples below, including zinc and iron. Suitable counter ions include those disclosed above and in Examples below. In some cases, the methods described herein may also include synthesis of a mixture of two or more nutrients with the same counter ion. Alternatively, the synthesis may include the same nutrient and a mixture of two or more counter ions.

Solubilizing the at least one nutrient and the at least one counter ion in at least one solvent includes at least partially solvating the at least one nutrient and the at least on counter ion. Suitable solvents may be chosen based upon the individual solubilities of the one or more nutrients and one or more counter ions. Suitable solvents may include aqueous solvent and non-aqueous solvents. In some cases, the solvent is water. In other cases, the solvent is combination of water and methanol.

It may be desirable to adjust the pH of the solution (i.e., a composition for transdermal delivery) containing at least one nutrient and at least one counter ion. Methods of measuring pH already well established in the art, such as indicator methods, metal-electrode methods, glass-electrode methods, or semiconductor sensor methods, may be used to measure the pH of the solution. The pH of the solution may be adjusted with methods already established in the art, including providing a source of OH⁻ or providing a source of H⁺. Suitable sources of OH⁻ include sodium hydroxide. Suitable sources of H⁺ include hydrochloric acid. In some cases, the pH may be adjusted to between about 1.0 to about 8.0. In other cases, the pH may be adjusted to about 1.6, about 4.0, or about 7.4. In some cases, the pH of the as-combined solution of nutrient and counterion and solvent may already be the desired pH and no pH adjustment is desired.

In another aspect of the method, the solvent is removed from the solution to produce a nutrient-counter ion salt (i.e., a composition for transdermal delivery). Methods of removing solvents from a solution already known in the art, including air drying, oven drying, lyophilization, and vacuum drying, may be suitable. Some cases, the nutrient-counter ion salt produced as a result of the solvent removal may include any combination of nutrients and counter ions disclosed herein, including iron ethyl maltol, iron maltol, iron kojic acid, iron propionate, iron hexanoate, iron oleate, zinc ethyl maltol, zinc maltol, zinc kojic acid, zinc propionate, or any combinations thereof. In some cases, the nutrient-counter ion salt may be a solid (i.e., crystalline, nanocrystalline, amorphous), a liquid, an emulsion (i.e., a microemulsion), a gel, a colloid, or any combinations thereof.

In one non-limiting example of the composition and method of making, the at least one nutrient is iron and the at least one counter ion is maltol. The iron and maltol may form a solution in water and the pH may be measured and adjusted, if necessary, to about pH 4. The water may be removed by lyophilization and iron maltol, a nutrient-counter ion salt, may be produced. Without wishing to be bound by theory, the pH of the aqueous solution of iron and maltol may be impactful to the stoichiometry of the nutrient-counter ion salt. In some cases, the stoichiometry of the nutrient-counter ion salt may result in optimized solubility of the nutrient-counter ion salt in solvents used in reservoir patches for the transdermal delivery of the nutrient to the skin of a subject in need. In other cases, the stoichiometry of the nutrient-counter ion salt may result in optimized transport of the nutrient-counter ion salt to the skin and/or within the skin.

In some cases, a vitamin D3 phosphate and pharmaceutically acceptable salts thereof and pharmaceutical compositions comprising the compound, composition for transdermal delivery, and methods for preparing the same, which are suitable for use in the transdermal patches disclosed herein, is detailed in International Publication Number WO 2022/084669, which is incorporated by reference herein, for any purpose.

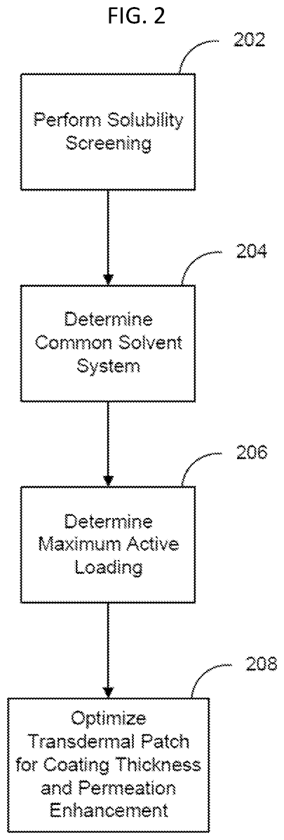

Referring to FIG. 2, a flowchart of non-limiting example steps of a method for optimization of a transdermal patch or composition for transdermal delivery is shown. A solubility screening may be performed at step 202 to identify or determine a common solvent system at step 204 for the at least one compound. A maximum active loading may be determined at step 206, where active loading may determine the maximum amount of the at least one compound (i.e., a composition for transdermal delivery) that may be included in the patch system. A transdermal patch or composition may be optimized for coating thickness or permeation enhancement at step 208. Other optimization may include optimizing adhesive properties, adhesive layer thickness, inclusion/percentage of permeation enhancers, and the like.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Unless otherwise specified or indicated by context, the terms “a”, “an”, and “the” mean “one or more.” For example, “a compound” should be interpreted to mean “one or more compounds.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

### EXAMPLES

### Example 1. Non-Limiting Zinc Example

### 1.1 Materials

Zinc chloride (conforms to Ph Eur, BP, USP) and concentrated hydrochloric acid, (HCl) (37%) were purchased from VWR International (UK). Zinc acetate, ammonium hydroxide, 2-dimethylaminoethanol, trifluoracetic acid, and triacetin were purchased from Merk (UK). Pyridine-2,6-dicarboxylic acid (PDCA), 4-(2-pyridylazol) resorcinol (PAR), Dionex™ PC10 post-column pneumatic delivery packages, and Dionex™ IonPac™ CS5A IC column and pre-column were purchased from Thermo Fisher Scientific (UK). Helium Gas was supplied by BOC (UK). Ethyl maltol (FCC, food-grade), maltol (FCC, food grade), sodium propionate (≥99%), propylene glycol (99%), and diethylene glycol monoethyl ether (Transcutol) (99%) and deuterated methanol (methanol-d4) were purchased from Sigma Aldrich (UK). Kojic acid (99%) was purchased from Alfa Aesar. Glycerol was purchased from Fisher Scientific (UK). Polyethylene glycol 400 (PEG400) was purchased from Fluka (UK). Phosphate buffered saline (Dulbecco A) (PBS) was purchased from Oxoid (UK). Liquid paraffin BP was purchased from Pinewood (UK).

### 1.2 Part 1. Co-Ordination Complexes Manufacture and Characterisation

### 1.2.1 Preparation of Co-Ordination Complexes

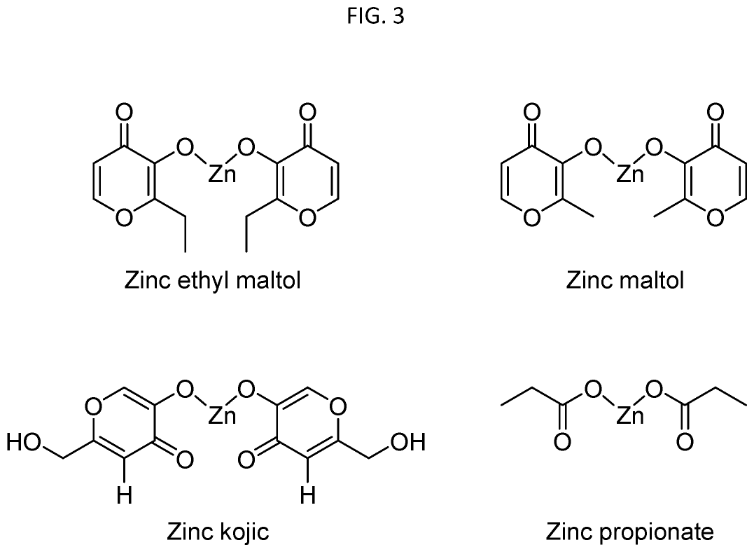

Zinc chloride was used as the zinc donor and it was combined with the organic ligands in excess (molar ratio of 1:2.5) to ensure it was possible to form the 1:2 Zinc:coordination complexes (FIG. 3). To make zinc ethyl maltol, zinc maltol, zinc kojic acid, and zinc propionate complexes, 40 mM of zinc chloride solution was prepared by weighing 545 mg into 100 mL with deionised water. Zinc chloride solution was then mixed with 300 mL of a 33.33 mM solution of the required ligand. The 300 mL stock solution at a concentration of 33.33 mM of the ligand was prepared by weighing 1401 mg, 1261 mg, 1421 mg, or 960.60 mg of ethyl maltol, maltol, kojic acid, or sodium propionate, respectively and adding it to 300 ml of deionised water (Error! Reference source not found.). The pH of the zinc-ligand solution was adjusted using 1 M NaOH or 0.1 M HCl to achieve the target pH. The pH of zinc ethyl maltol and zinc maltol was adjusted to 7.4, while the pH of zinc kojic acid was not adjusted. The pH of the zinc propionate complex was adjusted to 7.4, 6.4, 5.4, 4.4, 3.4, and 2.4. Aliquots of 8 ml of the final pH-adjusted mixtures were loaded into 27 mL glass vials and stored in the freezer for a minimum of 24 h. The samples were then placed in a Lyotrap freeze dryer (LTE Scientific Ltd, Oldham, Greater Manchester, UK) with a freeze-drying temperature of 218±5 K and a pressure of 0.03 mBar using an RV3 vacuum pump (Edwards Ltd, Burgess Hill, West Sussex, UK). The freeze-drying cycle length was 2-3 days. The freeze-dried products were visually checked for complete solvent evaporation and stored in the freezer until used.

TABLE 1
The preparation protocol of Zinc complexes. The solution
was prepared by mixing 100 mL of zinc chloride (solution
A) with 300 mL of solution B (ethyl maltol, maltol, kojic
acid, or sodium propionate) to achieve a molar ratio of
1:2.5 (Zinc chloride: counter ion).

| | Molar | Target | Mass of | Volume |
|---|---|---|---|---|
| | Mass | Mol- | sub- | of |
| | (g/ | arity | stance | solution |
| Material | mol) | (mM) | (mg) | (mL) |
| Zinc Chloride | 136.28 | 40 | 545.14 | 100 |
| (Solution A) | | | | |
| Ethyl Maltol | 140.14 | 33.3 | 1401.26 | 300 |
| (Solution B1) | | | | |
| Maltol | 126.11 | 33.3 | 1260.97 | 300 |
| (Solution B2) | | | | |
| Kojic Acid | 142.11 | 33.3 | 1420.95 | 300 |
| (Solution B3) | | | | |
| Sodium | 96.07 | 33.3 | 960.6 | 300 |
| Propionate | | | | |
| (Solution B4) | | | | |

### 1.2.2 Structural Characterisation of the Zinc Complexes

### 1.2.2.1 Nuclear Magnetic Resonance (NMR) Spectroscopy

¹H NMR spectra were generated using a DRX 400 (Bruker, UK) with Topspin software for data analysis. All zinc complexes were prepared by dissolving 8 mg in 0.8 mL of deuterated methanol (methanol-D₄). Proton NMR used a sweep width of 8250.825 Hz, an acquisition time of 4.0 s, an interpulse delay time of 1 s, and 16 scans.

### 1.2.2.2 Ultraviolent (UV) Scan

Solutions of zinc chloride, the ligands, and the complexes in methanol were prepared. UV scans were acquired for materials in the range of 250-750 nm using a plate reader Spark (Tecan, UK). An overlay of each complex with zinc chloride and its corresponding ligand was plotted to establish the shift in the λ max and consequently verify the production of the zinc-ligand complex.

### 1.2.3 Zinc Assay

### 1.2.3.1 Instrumentation

A 50×4 mm I.D. IonPac CG5A guard column connected to a 250×4 mm I.D. IonPac CS5A analytical column maintained at 60° C. was used for heavy metals separation. Data collection was handled with Waters Millennium chromatographic software. A two-eluent system was used for zinc determination with pyridine-2,6-dicarboxylic acid (PDCA) in the eluent buffer (7.0 mM PDCA, 66 mM KOH, 5.6 mM K₂SO₄, 74 mM HCOOH) employing a 1.2 mL/min flow rate and 4-(2-Pyridylazo) resorcinol (PAR) in the post-column derivatization buffer (0.2 mM PAR, 1.0 M 2-dimethylaminoethanol, 0.50 M NH₄OH, and 0.30 M CH₃COOH) at flow rate 0.6 mL/min. The PDCA eluent was filtered through a 0.2 μm Whatman filter paper. Both solvents were ultrasonicated for at least 20 min before use. Chromatographic analysis was performed on a high-performance liquid chromatography (HPLC) instrument including a 100 pump (Jasco, UK), 717 autosampler (Waters, UK), a 400 diode-array detector set at 530 nm (Waters, UK), and a PC10 pneumatic controller for post-column reagent (Dionex, UK).

### 1.2.3.2 Method Development

The ion chromatography method utilised the bidentate chelating agent, pyridine-2,6-dicarboxylic acid (PDCA), to form negatively charged 1:2 complexes with Zn²⁺ [1]. Once separated, the detection of zinc was performed using the colorimetric reagent, 4-(2-pyridylazo) resorcinol (PAR), mixed in excess with the effluent. PAR formed 1:2 complexes with Zn²⁺ in aqueous and aqueous-organic solutions. To optimise the post-column reaction robustness between Zn²⁺ and PAR the effect of different post-column reagent (PAR) flow rates on the absorbance of zinc acetate samples in HPLC was tested. The manufacturer recommended setting the flow rate of PAR at 0.6 mL/min. Thus, a higher flow of 0.8 mL/min and a lower flow rate of 0.4 mL/min were tested, using zinc acetate 10 g/mL as standard. The prepared zinc acetate was then injected in HPLC in triplicates for each flow rate.

### 1.2.3.3 Method Verification

Zinc acetate standard solutions were prepared by serial dilution in the range 1.25-20.0 g/mL (corresponding to 0.44-7.13 g/mL elemental zinc) and assayed employing five five-point calibration curves (three injections per each calibration point) run on three separate occasions. Linearity and range were determined by plotting the data points from all five calibration curves and calculating the regression line by the method of least squares, to obtain a value for the linear correlation coefficient (R) and the coefficient of determination (R²). The limit of detection (LOD) and the limit of quantitation (LOQ) were determined from all five calibration curves utilising Equations 1 and 2:

$$LOD = {3}^{*} SB / m \qquad ( Equation 1 )$$

$$LOQ = {10}^{*} SB / m \qquad ( Equation 2 )$$

where m is the slope of the regression line, and SB is the standard error of the regression. Intra-assay precision was determined from the relative standard deviations (RSD) of the analyte peak areas from three calibration curves of the same standard preparation run consecutively on the first occasion. Inter-assay precision was determined in the same way except from three calibration curves, each from a different standard preparation and run on three separate occasions. Accuracy was determined from five prepared sample concentrations in the range 13.40-19.58 g/mL zinc. The actual concentrations (x values) of the five sample concentrations were calculated from the response (y-values) and the equation of the regression line. The accuracy was then calculated utilising Equation 3.

$$\% Accuracy = A \times 100 T \qquad ( Equation 3 )$$

where A is the actual concentration (g/mL), and T is the theoretical concentration (g/mL) of the sample.

### 1.3 Part 2: Pre-Formulation Studies

### 1.3.1 Solubility Studies

Aliquots of zinc ethyl maltol, zinc maltol, and zinc propionate were added to a variety of solvents, including propylene glycol (PG), methanol, ethyl acetate, ethanol, hexane, polyethylene glycol (PEG400), diethylene glycol monoethyl ether, glycerol, triacetin, Miglyol, and liquid paraffin, to determine the visual solubility. Accurate measurement of the solubility was then repeated for some of the solvent systems by HPLC. In the HPLC solubility studies, an excess of the test agents was loaded into 2 mL of each test solvent. The solutions were left stirring overnight at 32° C. The visual appearance of the undissolved powder proved the saturation of the solutions. The drug suspensions were then centrifuged, the supernatant diluted, and each sample concentration was measured using HPLC.

### 1.3.2 Skin Permeation Studies

1.3.2.1 Method Development and Verification of Drug Recovery from Skin

To verify the percentage of drug that could be recovered from porcine skin, a 2.19±0.21 cm² piece of the skin was cut from a porcine ear. The piece of the skin was cut into small pieces using a scalpel. The cut piece of skin was then placed into glass vials, each of which was spiked with 80 μL of zinc chloride solution or 95 μL of zinc ethyl maltol dissolved in PG. The elemental zinc concentration of zinc chloride and zinc ethyl maltol in PG was 2.45 and 2.1 mg/mL, respectively. The vials containing the test agents and skin were left for 1 h for the agents to absorb into the tissues. Then, 4 mL of extraction fluid (mobile phase (PDCA)) was added to the skin. The samples were left for 1 h and transferred to Eppendorf tubes with the addition of one metal ball and placed into the TissuLyser II (Qiagen) for 20 min at 30 Hz. The samples were centrifuged (10 min at 13,000 rpm), filtered, and assayed. Control samples were prepared using the same procedure without the presence of the skin. The recovery study results were quantified using different calibration curves including: 1) zinc acetate calibration curve in water, 2) zinc chloride and zinc ethyl maltol in PG/PDCA calibration curve, and 3) zinc chloride and zinc ethyl maltol in skin matrix calibration curve.

### 1.3.2.2 Skin Permeation Studies

Porcine skin permeation studies were performed to compare the permeation of zinc chloride and the zinc ethyl maltol complex. The permeation of zinc chloride vs zinc ethyl maltol from PG saturated solutions (zinc chloride and zinc ethyl maltol solutions in PG was 2.45 and 2.10 mg/mL, respectively) and permeation of zinc chloride from PG statured solution vs zinc ethyl maltol from PEG400 saturated solutions were investigated (zinc ethyl maltol in PEG400 100 mg/mL). In addition, the permeation of the zinc ethyl complex from PEG400 saturated solution and PEG400:PEG1000 (8:2% w/w) was compared. For the PEG mixture, the zinc ethyl maltol donor was prepared by dissolving 60 mg of the complex in 3 mL of methanol and 1 mL of 80% w/w PEG400:20% w/w PEG 1000 then added. The solution was left overnight to allow the evaporation of methanol.

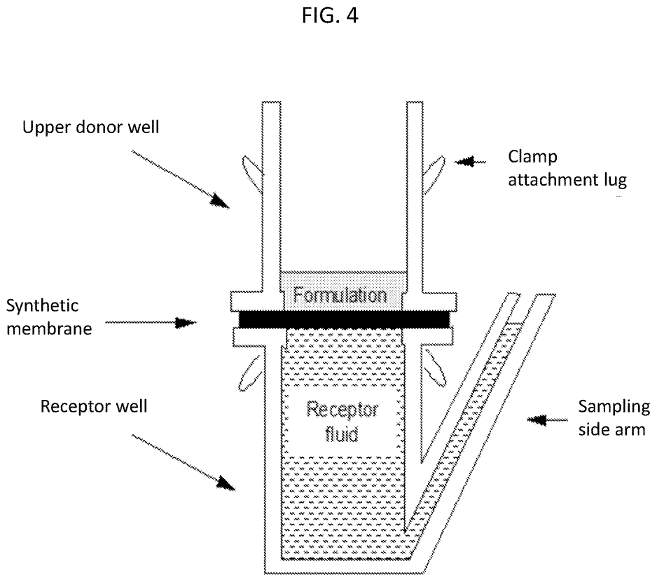

Permeation studies were conducted using previously validated Franz cell method based on the principles of the FDA, SUPAC-SS guidelines [FDA (CDER), 1997, Guidance for industry—SUPAC-SS Non-sterile Semisolid Dosage Form, Scale-up and post-approval changes: chemistry, manufacturing, and controls; in vitro release testing and in vivo bioequivalence documentation](FIG. 4). On the day of the experiment, the porcine skin was defrosted and either prepared as full-thickness sheets but removing the fat or dermatome to a 1 mm thickness (Nouvag, AG). The tissue was cut into 34 mm circles to allow an exposure area of 2.19±0.21 cm² and positioned between donor and receiver compartments of Franz diffusion cells. Each piece of skin was sealed between the two compartments using parafilm (Bemis Company, Neenah, WI). A 12 mm magnetic stir bar was placed into the receiver compartment of each Franz cell. The receiver compartment was loaded with 50 mM phosphate-buffered saline (pH 7.4). The cell sealing was verified by Franz cell inversion (6 times) and monitoring solvent back diffusion. The temperature of the Franz diffusion was set at 37° C. in a water bath to reach a skin temperature of 32° C. After 1 h equilibration at 37° C. in the water bath, 1 mL of receiver fluid was withdrawn (time point=0 min), and a fresh receiver solution was replaced into the cell to keep the volume consistent. Each formulation (1 mL) was applied in the donor compartment that was then covered with parafilm to avoid evaporation. After 24 h, the receiver fluid was collected and analysed by HPLC.

At the end of the permeation studies, the Franz cells were dismantled. The donor solutions were collected from each cell and transferred into 5-mL glass bottles containing 2 mL of PDCA as extraction fluid. This step was followed by wiping the skin surface and the donor compartment with two wet (wetted with extraction fluid) and then two dry cotton buds. The skin's permeation area was then cut into small pieces by scalpels and placed into a 5 mL glass bottle containing 4 mL of extraction fluid. All skin samples were kept at the bench with extraction fluid for 1 hour. This step was followed by separating each vial's content (cut skin and extraction fluid) into 2 mL Eppendorf tubes that were then loaded in TissuLyser machine for 20 minutes. Samples were then centrifuged for 15 minutes at 13,000 rpm. The content of each Eppendorf tube was filtered using 0.45 m syringe filters, collected, and analysed by HPLC.

### 1.4 Patch Development and Performance Testing

### 1.4.1 Development of Zinc Ethyl Maltol in Adhesive Patch

Pre-formulation studies were performed to determine the compatibility of zinc ethyl maltol with a variety of adhesives that could be used to manufacture a transdermal patch (Table 2). The compatibility of the zinc complexes with the patch excipients was evaluated by examining the clarity of the mixture upon mixing. The adhesive solutions that did not dissolve the drug with or without the inclusion of co-solvents were excluded from the study. Then with the compatible mixtures, the maximum drug loading in the adhesive following solvent evaporation was evaluated by mixing the drug, adhesive, and co-solvents until a clear solution was achieved at a different drug:adhesive ratios and then placing a drop of solution onto patch backing sheet where it was left to dry for 3-4 h. The formed film on the surface of the backings was examined for consistency and film clarity (clear films showed the drug was not crystalising in the adhesive). Following patch optimisation, the optimised drug-adhesive solutions were cast in 10×10 cm² trays made of the patch backing (3M™ Scotchpak™ Polyester Backing Film Laminate 9730, 3M, Germany). An adequate volume of the adhesive was applied to achieve a target coating level of 8 mg/cm² (equivalent to 80 g/m²). Following complete drying of the solvents, the adhesive layer was covered by an M3 Release liner (SCOTCHPAK 9755), compressed, and then cut using a 34 mm circular manual die cutter (SCP Super Cutting Press).

TABLE 2
Type of patch adhesives and their properties.

| Type of | Name of | | Solvent |
|---|---|---|---|
| adhesive | adhesive | Properties | Compatibility |
| Acrylates | DURO- | No | Ethyl |
| copolymer | TAK | functional | acetate/ |
| (pressure | 87-9301 | groups | methanol |
| adhesives) | (Henkel) | 36.5% | PG |
| | | solids | Isopropanol |
| | | | Propanol |
| | DURO- | —OH | Ethyl |
| | TAK | functional | acetate |
| | 87-2516 | group | Ethyl |
| | (Henkel) | Contains | acetate/PG |
| | | cross- | Ethyl |
| | | linker | acetate/ |
| | | 41.5% | Methanol |
| | | solids | |
| | DURO- | —COOH | Hexane |
| | TAK | functional | |
| | 87-2052 | group | |
| | (Henkel) | Contains | |
| | | cross- | |
| | | linker | |
| | | Contains | |
| | | vinyl | |
| | | acetate | |
| | | 47.5% | |
| | | solids | |
| Polyiso- | Oppanol | Neutral | Hexane |
| butene | B12 | Low | |
| | (BASF) | molecular | |
| | | weight | |

### 1.4.2 Zinc Ethyl Maltol Adhesive Patch Manufacturing

The zinc ethyl maltol patch was made using the DURO-TAK 87-9301 adhesive (Table 3). Zinc ethyl maltol was dissolved in methanol at a concentration of 16 mg/mL. The adhesive was diluted in ethyl acetate in a ratio of 1% v/v of adhesive to 2% v/v ethyl acetate. The formulation was prepared by mixing the dry equivalent of 59.2 mg of zinc ethyl maltol, 666 mg of DURO-TAK 87-9301, and 74 mg of Transcutol (Table 3). The patch formulations were then cast into an in-house rectangular tray made from backing film material measuring 10×10 cm. The mixture was left to dry overnight. The laminate was then applied to the adhesive, and circles of 34 mm diameter were cut using a dye cutting machine.

TABLE 3
Zinc ethyl maltol adhesive patch composition for
deposition and transdermal permeation studies.

| | Ingredients | | Wet | | Dry |
|---|---|---|---|---|---|
| | (Per 100 | Weight | weight | Ingredient | weight |
| Patch | cm²) | (mg) | (%) | conversion | (%) |
| F1 | Zinc | 59.20 | 0.84% | 59.20 | 7.4% |
| | Ethyl | | | mg | |
| | Maltol | | | | |
| | DURO- | 666.00 | 9.43% | 666.00 | 83.3% |
| | TAK | | | mg | |
| | 87-9301 | | | | |
| | Transcutol | 74.00 | 1.05% | 74.00 | 9.3% |
| | | | | mg | |
| | Ethyl | 3337.40 | 47.25% | 3.70 | |
| | Acetate | | | mL | |
| | Methanol | 2926.70 | 41.44% | 3.70 | |
| | | | | mL | |
| | Sum | 7063.30 | 100% | 799.20 | 100% |
| | | mg | | mg | |

### 1.4.3 Zinc Ethyl Maltol in Adhesive Patch Transdermal Permeation and Deposition Studies

The skin permeation and deposition studies were performed as per Section 2, but the skin was glued to the receiver chamber of the Franz diffusion cell with the stratum corneum facing the donor compartment to allow the patches to be placed on the surface of the skin. The receiver fluid was PEG400:saline pH 7.4 (80% v/v: 20% v/v).

### 1.4.4 Development of Zinc Ethyl Maltol Reservoir Patch

Pre-formulation studies were completed to optimise the solubility of zinc ethyl maltol using a variety of co-solvents that could be applied to the skin, such as Transcutol and ethanol. Polymers such as PEG 1000, Natrsol M250, and PVP were used to aid the solubilisation process. The compatibility of the complex with the solvents was evaluated by examining the clarity of the mixture. Following the optimisation of the complex solubility, the reservoir patch was developed using the backing layer, drug in solution, and liner release layer. The backing used was 3M™ Scotchpak™ Polyester Backing Film Laminate 9730. Different liner release membranes were investigated including, regenerated cellulose (12-14 k molecular weight cut-off Medical International UK), nylon (0.20 μm, Whatman, UK), and CoTran 9728 (3M, Germany). A 200 μL aliquot of zinc ethyl maltol complex in solution was applied on the liner release membrane, then covered by the backing layer and all layers thermally sealed (HeatPress machine HP230B).

### 1.4.5 Zinc Ethyl Maltol Reservoir Patch Manufacturing

The zinc ethyl maltol reservoir patch was made by dissolving 60 mg of zinc ethyl maltol in 3 mL of methanol, followed by adding 1 mL of PEG 400:PEG1000 (8% w/w: 2% w/w). The solution left to evaporate overnight to form a supersaturated solution (Table 4). This solution was then loaded into the reservoir patch.

TABLE 1
Zinc ethyl maltol reservoir patch composition for
deposition and transdermal permeation studies.

| | | | Wet | | Final |
|---|---|---|---|---|---|
| | | Weight | weight | Ingredient | weight |
| Patch | Ingredient | (mg) | (%) | conversion | (%) |
| F2 | Zinc | 60 | 1.75% | 60 | 5.7% |
| | Ethyl | | | mg | |
| | Maltol | | | | |
| | PEG | 800 | 23.3% | 800 | 75.5% |
| | 400 | | | mg | |
| | PEG | 200 | 5.8% | 200 | 18.9% |
| | 1000 | | | mg | |
| | Methanol | 2373 | 69.1% | 3 | NA |
| | | | | mL | |
| | Sum | 3433 | 100% | 1060 | 100% |

### 1.4.6 Zinc Ethyl Maltol Reservoir Patch Transdermal Permeation and Deposition Studies

Permeation and deposition studies were conducted for F2 patch, with three different liner release membranes, regenerated cellulose, nylon, and CoTran 9728. The patch was produced by applying 200 μL of the supersaturated solution on the backing (3M™ Scotchpak™ Polyester Backing Film Laminate 9730, 3M, Germany). This was followed by applying thermal sealing using a stainless steel circular die (5.2 cm diameter) and thermal press at 90° C. for 1.5 minutes. Transdermal permeation and deposition studies are conducted following the same procedure referenced above.

### 1.5 Results

### 1.5.1 Part 1: Zinc Coordination Complexes Manufacture and Characterisation

### 1.5.1.1 Preparation of the Zinc Complexes

All the complexes were successfully prepared as described in Table 1 (Section 1.2.1), but the zinc propionate solutions a pH of 7.4 and 6.4 were excluded as they started to precipitate upon pH adjustment. All zinc complexes batches were generated freeze-dried white powder. The percentage yield of zinc ethyl maltol, zinc maltol, zinc kojic, and zinc propionate was 77%, 83%, 76.29%, and 90%, respectively. The percentage of the elemental zinc was calculated based on zinc chloride and complexes' molecular weight (Table 1). The elemental zinc percentage in zinc ethyl maltol, zinc maltol, zinc kojic, and zinc propionate was 23%, 26%, 23%, and 34%, respectively.

### 1.5.1.2 Characterisation of Zinc Complexes

### NMR Analysis

NMR analysis was conducted to confirm the structure of the synthesised zinc complexes. As predicted, the NMR of ethyl maltol showed a triplet at 1.23 ppm, quartet at 2.75 ppm, doublet at 6.38 ppm, and a doublet at 7.96. In all NMR spectra, peaks around 2 ppm, 3.3 ppm, and 4.7 ppm are related to acetone, methanol D₄, and water. The NMR spectra of zinc ethyl maltol showed a similar splitting pattern and chemical shifts, but the small downfield shifts of the CH₂ and CH groups in the NMR indicated the formation of zinc ethyl maltol complex. Similarly, the NMR spectra of maltol and zinc maltol showed the chemical shifts from 2.36 to 2.45 ppm (singlet), from 6.40 to 6.62 ppm (doublet), and from 7.95 ppm to 8.03 ppm (doublet) that indicated complex formation. The NMR spectra of the proposed zinc kojic acid showed no chemical shift and similar chemical pattern, suggesting that the complex was not formed. The NMR spectra of sodium propionate and zinc propionate (pH 5.4) showed a chemical shift from 1.08 to 1.10 ppm (triplet) and from 2.15 to 2.25 ppm (quartet). Chemical shifting was also noticed with zinc propionate complexes produced at different pH values (Error! Reference source not found.). In conclusion, the NMR spectra confirmed the formation of zinc ethyl maltol, zinc maltol, and zinc propionate complexes only.

TABLE 5
Comparison between the splitting pattern and chemical
shift (ppm) of sodium propionate and zinc propionate
complex prepared at different pH values; 5.4, 4.4, and 3.4.

| | Sodium | |
|---|---|---|
| | Propionate | |
| Splitting | (ppm) | |
| | | Zinc Propionate - |
| | | pH 5.4 (ppm) |
| Triplet | 1.081 | 1.104 |
| Quartet | 2.154 | 2.252 |
| | | Zinc Propionate - |
| | | pH 4.4 (ppm) |
| Triplet | 1.08 | 1.103 |
| Quartet | 2.15 | 2.251 |
| | | Zinc Propionate - |
| | | pH 3.4 (ppm) |
| Triplet | 1.08 | 1.101 |
| Quartet | 2.15 | 2.252 |

### UV Analysis

A UV scan was conducted for zinc complexes made with ethyl maltol, maltol, and kojic as the complexing agents each contained a UV chromophore that should shift in the λₘₐₓ when the complex with zinc is formed. Sodium propionate and zinc propionate were not tested due to the lack of a UV chromophore in their structure (FIG. 1). The UV results supported the NMR data in that they suggested the formation of zinc ethyl maltol, zinc maltol through a shift in λₘₐₓ. The lack of change in the λₘₐₓ in zinc kojic and kojic acid spectra indicated that the proposed complex was not formed.

### Development of Zinc Assay

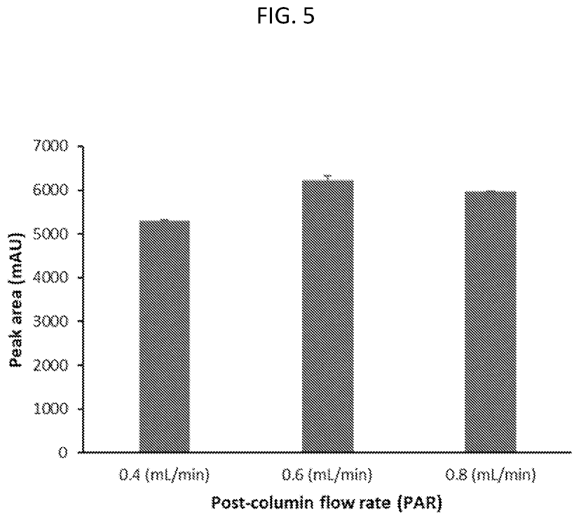

The robustness of the post-column PAR reaction flow was studied by measuring zinc acetate sample peak area (10 g/mL) whilst changing the post-column reagent flow rate to 0.4, 0.6, or 0.8 mL/min. The number of injections of zinc acetate was 3 for each flow rate. The peak area for zinc acetate changed significantly (p<0.05, t-test) as the post-column flow rates varied (FIG. 5). The sample's peak areas were 5298±30, 6218±107, and 5973±3 mAU, for a PAR flow rate of 0.4, 0.6, and 0.8 mL/min, respectively. The results confirmed the optimal flow rate of 0.6 mL/min as it was associated with the sample's highest absorbance (FIG. 5).

### Verification of the Zinc Assay

The retention time derived from the 5 calibration curves was 9.06±0.17 min (n=20). The peak symmetry was 1.08±0.07 (n=20). Two HPLC instruments were used in this project, Agilent 1200 HPLC and Waters HPLC systems. The calibration curve using the Agilent system with an R² value of 0.9992. The assay was linear between 0.44 and 7.13 g/mL, with a LOD of 0.29 g/mL and a LOQ of 0.86 g/mL (

Table). The calibration curve generated using the Waters system showed good linearity with an R² value of 0.9997 derived for the line of regression. The assay was linear between 0.44 and 7.13 g/mL, with a LOD of 0.2 g/mL and a LOQ of 0.6 g/mL, respectively.

The % relative standard deviation (RSD) values of all injections from each of the five calibration curves and as expected, the % RSD values started to increase with the lower tested concentrations (Tables 6 and 7). The % RSD values for inter-day were calculated based on the injections of elemental zinc concentration of 2.60 (g/mL) from the three calibration curves derived from three different standard preparations and run non-consecutively on occasions 1-3. The reported % RSD for the interday variability was 1.8% (Table 7). Similarly, the intraday variability was calculated based on the middle elemental zinc concentration (2.60 g/mL), which reported a % RSD of 0.7% (Table 6). Waters HPLC's precision showed % RSD of 1.6% and 1.3%, for intraday and interday variability, respectively (Table 8). The Accuracy of the 5 prepared samples was 98.82±2.80, which is within the level of the ICH guidelines (Table 9).

TABLE 2
Illustrate Intra-day assay calibration curves 1-3, mean
peak areas and % relative standard deviations (% RSD), n = 3.
Intraday calibration curve

| Zn²⁺ Conc. | | | | | | | | |
|---|---|---|---|---|---|---|---|---|
| (μg/mL) | Curve 1 | % RSD | Curve 2 | % RSD | Curve 3 | % RSD | Curve 1-3 | % RSD |
| 7.13 | 5264.1 | 0.85 | 5416.4 | 1.16 | 5422.8 | 0.58 | 5367.8 | 1.4 |
| 5.34 | 3773.7 | 3.01 | 3760.6 | 2.25 | 3786.6 | 1.11 | 3773.6 | 0.3 |
| 3.56 | 2469.4 | 3.44 | 2676.3 | 2.58 | 2296.4 | 2.28 | 2480.7 | 6.3 |
| 2.60 | 1720.2 | 4.32 | 1718.7 | 2.49 | 1694.6 | 2.02 | 1711.2 | 0.7 |
| 1.70 | 772.2 | 14.33 | 825.9 | 7.69 | 889.8 | 4.89 | 829.3 | 5.8 |
| 0.89 | 376.8 | 22.23 | 222.7 | 18.61 | 242.8 | 34.01 | 280.8 | 24.3 |
| 0.44 | 101.1 | 39.05 | 169.3 | 12.67 | 66.0 | 48.19 | 112.1 | 38.2 |

TABLE 3
Illustrate inter-day assay calibration curves 1-3, 4, and 5,
mean peak areas and % relative standard deviations (% RSD), n = 3.
Interday calibration curve

| Zn²⁺ Conc. | | | | | | | | |
|---|---|---|---|---|---|---|---|---|
| (μg/mL) | Curve 1 | % RSD | Curve 2 | % RSD | Curve 3 | % RSD | Curve 1-3 | % RSD |
| 7.13 | 5367.8 | 1.4 | 5395.3 | 1.1 | 5375.7 | 0.4 | 5379.6 | 0.2 |
| 5.34 | 3773.6 | 0.3 | 3982.8 | 1.0 | 3835.4 | 0.3 | 3863.9 | 2.3 |
| 3.66 | 2480.7 | 6.3 | 2582.4 | 1.0 | 2433.9 | 0.5 | 2499.0 | 2.5 |
| 2.60 | 1711.2 | 0.7 | 1637.8 | 1.2 | 1691.4 | 0.8 | 1680.1 | 1.8 |
| 1.70 | 829.3 | 5.8 | 768.1 | 0.5 | 983.0 | 1.6 | 860.1 | 10.5 |
| 0.89 | 280.8 | 24.4 | 167.9 | 5.1 | 618.8 | 4.2 | 355.8 | 53.8 |
| 0.44 | 112.1 | 38.2 | NA | NA | 314.2 | NA | NA | NA |

TABLE 8
Validation parameters compared to ICH guidelines.
(Agilent HPLC)

| Validation | ICH | Elemental |
|---|---|---|
| parameter | level/limit | Zinc |
| System suitability | | |
| Linearity range (μg/mL) | — | 0.4-7.1 |
| Linearity (R², n = 3) | >0.99 | ✓ |
| | | (0.9992) |
| Peak symmetry, As | <2, Ideal | ✓ |
| (n = 3 ± SD) | As = 1 | (1.08 ± 0.07) |
| LOD (μg/mL) | — | 0.28 |
| LOQ (μg/mL) | — | 0.86 |
| Precision | | |
| Intra-day variability | <2% | 0.7 |
| (repeatability, % CV, | | |
| for Zn concentration | | |
| 2.60 μg/mL) | | |
| Inter-day variability | <2% | 1.8 |
| (intermediate | | |
| precision, % CV, for | | |
| Zn concentration 2.60 | | |
| μg/mL) | | |

TABLE 9
Validation parameters compared to ICH guidelines.
(Waters HPLC)

| Validation | ICH | Elemental |
|---|---|---|
| parameter | level/limit | Zinc |
| System suitability | | |
| Linearity range (μg/mL) | — | 0.4-7.1 |
| Linearity (R², n = 3) | >0.99 | ✓ (0.9997) |
| LOD (μg/mL) | — | 0.20 |
| LOQ (μg/mL) | — | 0.60 |
| Accuracy % | 95-105% | 98.82 ± 2.80 |
| (n = 3 ± SD) | | |
| Precision | | |
| Intra-day variability | <2% | 1.6 |
| (repeatability, % CV) | | |
| Inter-day variability | <2% | 1.3 |
| (intermediate precision | | |
| % CV) | | |

### 1.5.2 Part 2. Pre-formulation Studies

### 1.5.2.1 Solubility Studies

The solubility of zinc chloride, and zinc ethyl maltol, zinc maltol, and zinc propionate complexes were tested in various solvents. The best solvents for each material are illustrated in Table 10.

TABLE 10
Illustrated most suitable solvents for each compound.
The symbol (*) indicates the visual solubility of the
compound.

| | | Elemental |
|---|---|---|
| | | Zinc |
| | | Solubility |
| | | (mg/mL) |
| | | evaluated by |
| Compound | Solvent | HPLC |
| Zinc | PG | 2.7 |
| Chloride | PEG400 | ~2.7* |
| Zinc Ethyl | PG | 2.1 |
| Maltol | Transcutol | 6.0 |
| | PEG400 | 6.6 |
| | PEG400/ | 28.8* |
| | PEG1000 | |
| | (80% w/w: | |
| | 20% w/w | |
| Zinc Maltol | Transcutol | 9.3* |
| | PEG400 | 21.0* |
| Zinc Propionate | PG | |
| of over | Transcutol | |
| the wide | PEG400 | 11.8* |
| range of pHs | Ethyl Acetate | (for all solvents) |

### 1.5.2.2 Skin Permeation Studies

Method Development and Zinc Recovery from the Skin

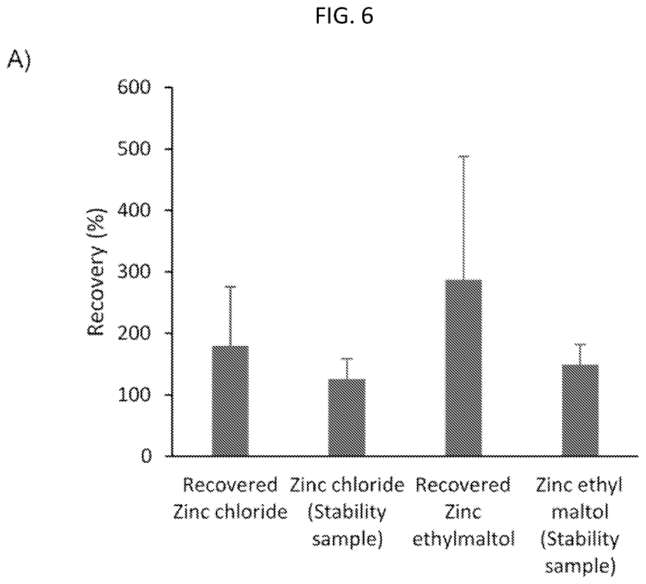

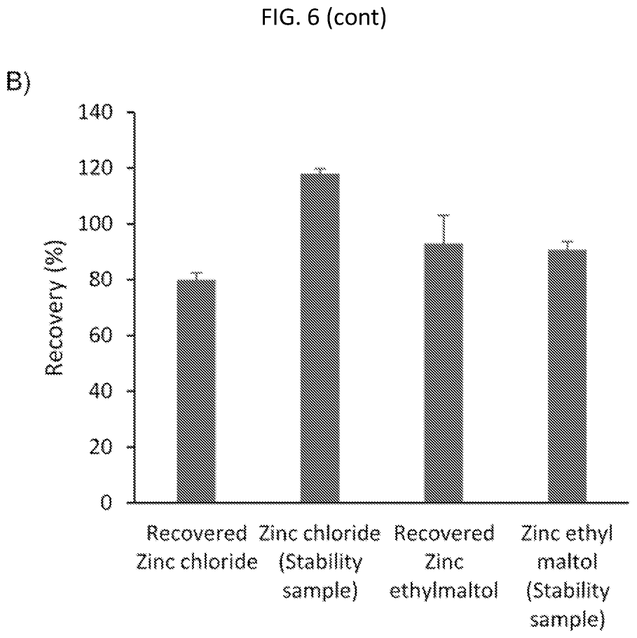

A new calibration curve of zinc chloride in PG/PDCA and zinc ethyl maltol in PG/PDCA was developed to account for PG:PDCA ratio in the recovery study. In addition, zinc chloride and zinc ethyl maltol calibrations using the skin matrix was constructed to consider the influence of the skin proteins. When comparing the recovery experiment for zinc chloride and zinc ethyl maltol from porcine skin the results were more consistent when the skin calibration curve was used (FIG. 6). Using the skin calibration curve a recovery of 80±2% and 93±10% for zinc chloride and zinc ethyl maltol, respectively. No correction was applied to the data to account for the losses observed in the recovery studies as they were considered not to have a significant impact on the subsequent studies. There was some detection of zinc in the blank samples of the skin matrix also indicates endogenous zinc in the skin.

### Zinc Complex Permeation and Deposition

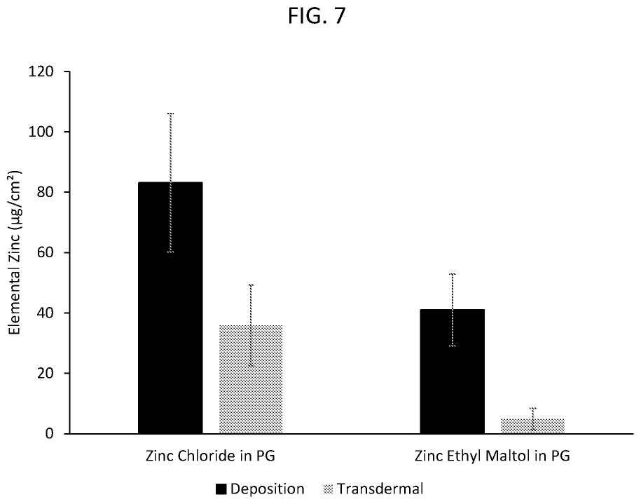

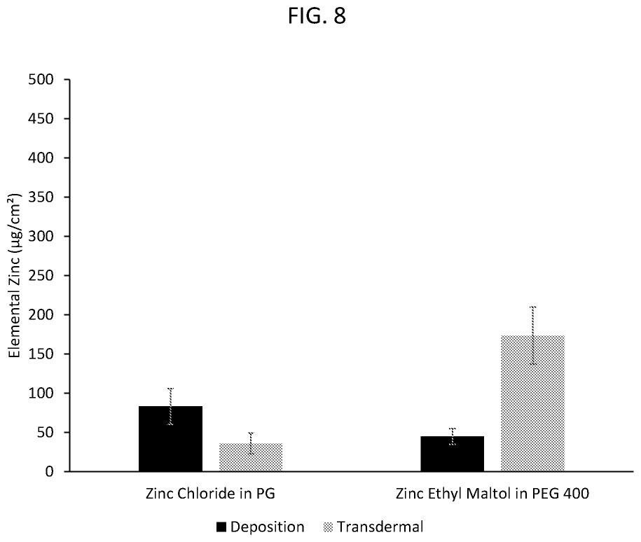

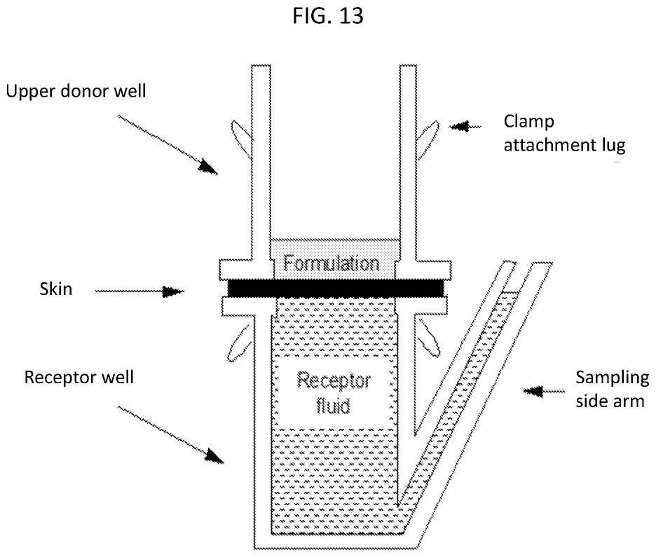

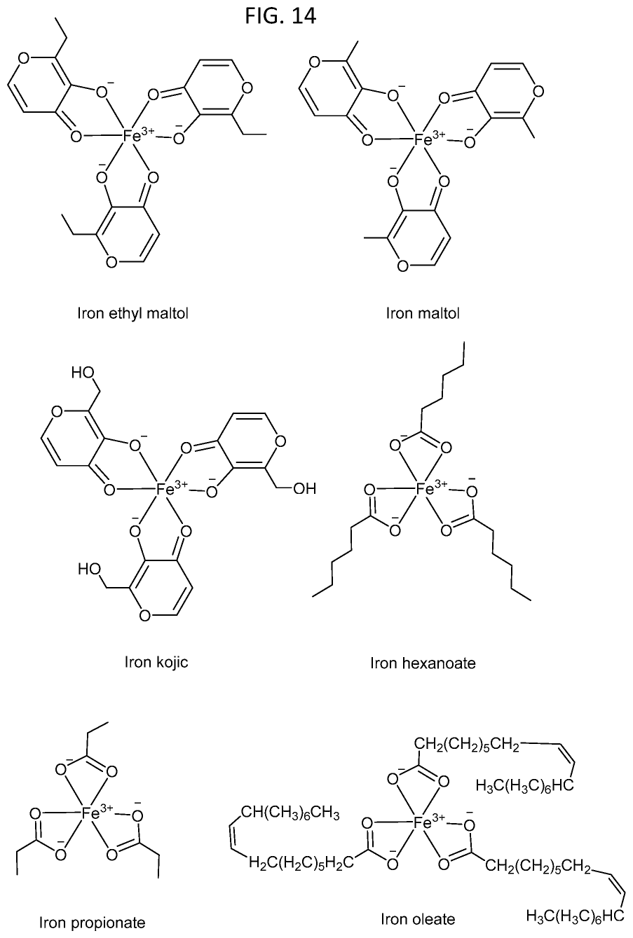

A skin permeation study was performed by applying 1 mL of zinc chloride (elemental zinc 2.45 mg/mL) and zinc ethyl maltol (elemental zinc 2.10 mg/mL) in PG solutions. After 24 h, deposition studies showed that both zinc chloride and zinc ethyl maltol showed elemental zinc skin deposition of 83.14 g/cm² and 41.00 g/cm², respectively (FIG. 7). Similarly, both compounds permeated the skin; however, zinc chloride showed superior transdermal permeation (35.82 g/cm²) compared to zinc ethyl maltol (4.85 g/cm², FIG. 13). Switching the zinc ethyl maltol vehicle to PEG400 increased the elemental zinc deposition from zinc ethyl maltol to 44.84 ag/cm² and the transdermal permeation increased to 173.59 ag/cm² (FIG. 8). These studies showed that elemental zinc in the form of a complex, i.e., zinc ethyl maltol has 4.8-fold transdermal permeation compared to zinc chloride. The higher permeation was achieved when PEG400 was included in the formulation because zinc ethyl maltol has higher solubility on PEG400 as a solvent.

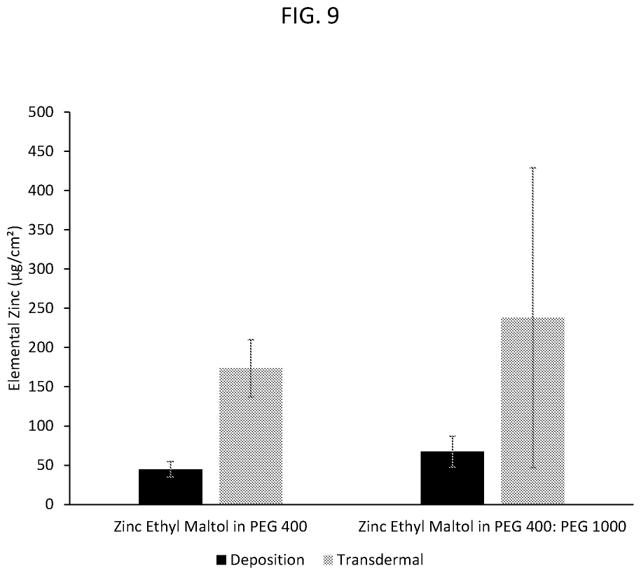

To try and increase the solubility of the zinc complex further a supersaturated solution was engineered. Initially, methanol was used as a co-solvent to solubilise zinc ethyl maltol and a combination of PEG400 (solvent) and PEG1000 (precipitation inhibitor) (8% w/w:2% w/w) was added to the complex. Finally, methanol was evaporated under stirring to yield a supersaturated solution. The deposition and transdermal permeation of zinc ethyl maltol supersaturated solution in PEG400:PEG1000 was then performed. The results showed that 237.93 ag/cm² of elemental zinc from the zinc ethyl maltol complex in PEG400:PEG1000 was permeated through the skin (FIG. 9). In conclusion, zinc ethyl maltol in both PEG400 and PEG400:PEG1000 showed a significant enhancement of transdermal permeation. Therefore, these systems were taken forward for patch development.

### 1.5.3 Part 3. Patch Development

### 1.5.3.1 Zinc Ethyl Maltol Adhesive Suitability

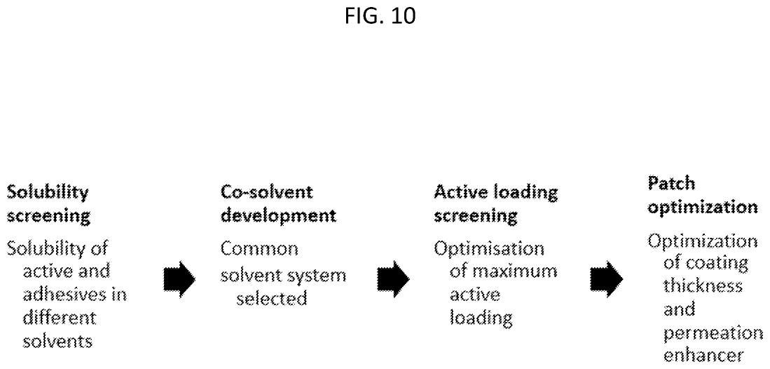

The patch development process was performed using a systematic process (FIG. 10). Initially, a common solvent for both drug and polymers was identified. This was followed by optimising drug loading, adhesive properties, adhesive layer thickness, and inclusion/percentage of permeation enhancers.

### Zinc Ethyl Maltol Adhesive Suitability Screening

The compatibility of DURO-TAK 87-2052 and 87-2516 was tested with PEG400 and Transcutol (Table 11). The generated results showed that both adhesives were soluble in Transcutol, but not PEG400. The subsequent step was the addition of co-solvents such as methanol, ethanol, and propanol. However, the formulation did not show acceptable miscibility once the zinc ethyl maltol complex was added (Table 11). Therefore, these adhesives were unsuitable for patch production.

TABLE 11
DURO-TAX 87-2052 and 87-2516 suitability screening.

| Zinc | DURO- | DURO- | | | |
|---|---|---|---|---|---|
| Ethyl | TAK | TAK | | | |
| Maltol | 87-2052 | 87-2516 | | Solvents | Solubility |
| — | 0.5 g | — | | PEG 400 | Not |
| | | | | 0.25 g | Soluble |
| — | — | 0.6 g | | PEG 400 | Not |
| | | | | 0.25 g | soluble |
| — | 0.5 g | — | Transcutol | — | Soluble |
| | | | 0.25 g | | |
| — | — | 0.6 g | Transcutol | — | Soluble |
| | | | 0.25 g | | |
| 3.5 mg | 100 μL | — | Transcutol | — | Not |
| | | | 10 μL | | soluble |
| 5.5 mg | 100 μL | — | Transcutol | Methanol | Not |
| | | | 20 μL | 250 μL | Soluble |
| 5.5 mg | — | 100 μL | Transcutol | Methanol | Not |
| | | | 20 μL | 250 μL | Soluble |
| 2.3 mg | 100 μL | — | Transcutol | Ethanol | No |
| | | | 10 μL | 200 μL | Soluble |
| 4.9 mg | 100 μL | — | Transcutol | Propanol | Not |
| | | | 20 μL | 250 μL | Soluble |

The compatibility of DURO-TAK 87-9301 with common solvent for zinc ethyl maltol was then tested in a range of drug loading from 2.5% to 10%, with and without the presence of Transcutol (Table 12). A clear solution with a drug loading of 7.4% w/w dry weight was generated with the addition of Transcutol as a co-solvent/permeation enhancer. This formulation was then applied to the backing to produce a patch (F1 patch, Table 3). Transdermal permeation and deposition of F1 patch and an existing commercial L-lysine zinc transdermal patch were tested. However, both formulations showed no deposition and no transdermal permeation. Therefore, the next step was developing and testing reservoir patches.

TABLE 12
DURO-TAK 9301 suitability screening.

| | Zinc | DURO- | | |
|---|---|---|---|---|
| | ethyl | TAK | | |
| Drug | maltol | 9301 | Transcutol | |
| loading | (mg) | (mg) | (mg) | Solubility |
| 10% | 10.0 | 90.0 | — | Not Soluble |
| 7.5% | 7.5 | 92.5 | — | Not Soluble |
| 5% | 5.0 | 95.0 | — | Not Soluble |
| 2.5% | 2.5 | 97.5 | — | Not Soluble |
| 7.4% | 10.0 | 112.5 | 12.5 | Soluble |

### 1.5.3.2 Development of Zinc Ethyl Maltol Reservoir Patch

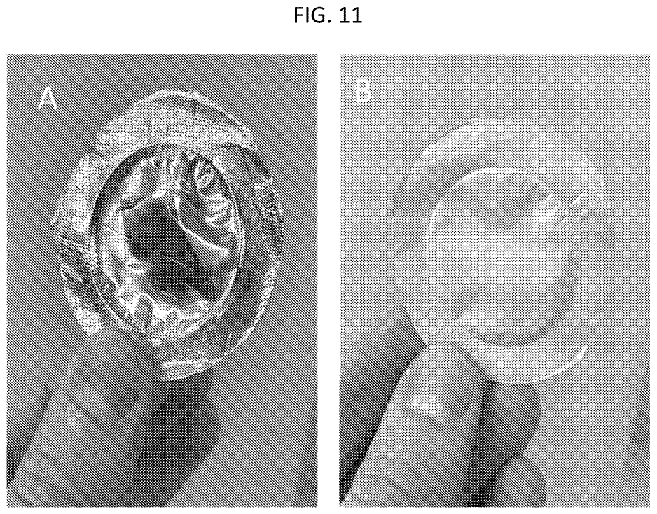

The solubility of zinc ethyl maltol in a range of co-solvents was tested (Table 13). The maximum solubility of zinc ethyl maltol complex was 30 mg/mL in PEG400. The solubility of zinc ethyl maltol was then tested in a range of different ratios of PEG 400 to PEG 1000 loaded with different amounts of the complex (Table 14). The only combination that produced a stable supersaturated solution following methanol evaporation was 60 mg of zinc ethyl maltol in 1 mL of PEG400:PEG 1000 (8% w/w:2% w/w), which was used for the development of the reservoir patch (F2, Table 4, FIG. 11).

TABLE 14
Zinc ethyl maltol screening in different combination of
PEG400 and PEG 1000 for reservoir patch development.

| Zinc | | | |
|---|---|---|---|
| Ethyl | | | |
| Maltol | | | |
| (mg) | Methanol | 2^{nd} Component | Solubility |
| 60 | 3 mL | 1 mL of PEG400:PEG1000 | Not soluble |
| | | (2% w/w: 8% w/w) | |
| 60 | 3 mL | 1 mL of PEG400:PEG1000 | Not soluble |
| | | (6% w/w: 4% w/w) | |
| 80 | 3 mL | 1 mL of PEG400:PEG1000 | Not soluble |
| | | (6% w/w: 4% w/w) | |
| 100 | 3 mL | 1 mL of PEG400:PEG1000 | Not soluble |
| | | (6% w/w: 4% w/w) | |
| 120 | 3 mL | 1 mL of PEG400:PEG1000 | Not soluble |
| | | (6% w/w: 4% w/w) | |
| 60 | 3 mL | 1 mL of PEG400:PEG1000 | Soluble |
| | | (8% w/w: 2% w/w) | |
| 80 | 3 mL | 1 mL of PEG400:PEG1000 | Not soluble |
| | | (8% w/w: 2% w/w) | |
| 100 | 3 mL | 1 mL of PEG400:PEG1000 | Not soluble |
| | | (8% w/w: 2% w/w) | |
| 120 | 3 mL | 1 mL of PEG400:PEG1000 | Not soluble |
| | | (8% w/w: 2% w/w) | |

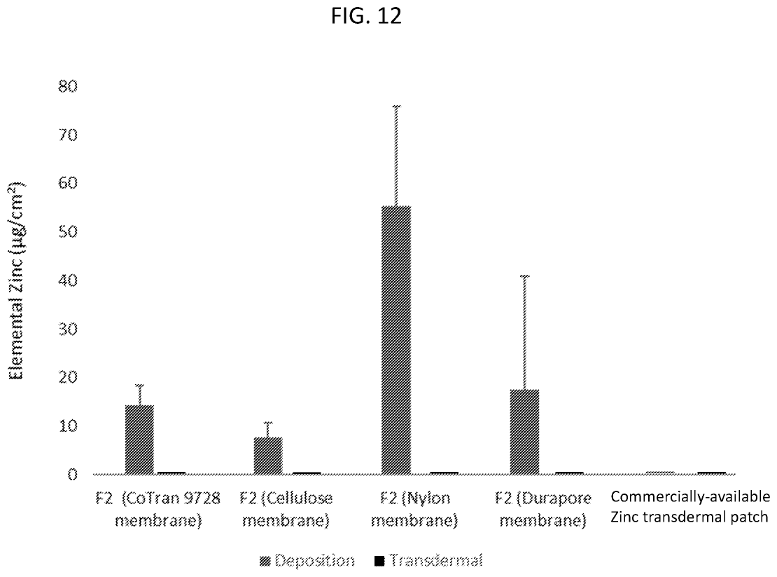

The transdermal permeation and deposition studies were conducted for the F2 reservoir patch with four different liner release membranes. In this study, a commercially available zinc transdermal patch was used as a comparator. The results showed that elemental zinc was not deposited or permeated the skin from the commercially available zinc transdermal patch. The zinc ethyl maltol complex did not permeate through the skin, but a total of 55.3±20.6 μg/cm² of zinc could be delivered into the skin (FIG. 12). Using a 1-inch patch for 24 hours, the F2 nylon patch would be predicted to deliver 357 μg/cm².

### 1.6 Conclusions

Zinc maltol and zinc ethyl maltol complexes were generated, and their formation was confirmed using NMR and UV spectroscopy. Solutions of zinc ethyl maltol in PG (2.1 mg/mL) and PEG (6 mg/mL) allowed the permeation of 41 and 173.6 μg/cm² of elemental zinc respectively. To maximise zinc complex loading into solution a novel supersaturated solution based on PEG400/PG6000 was engineered to achieve 28.8 mg/mL of elemental zinc. When tested in porcine skin 237.93 μg/cm² of elemental zinc passed through the skin. Two types of formulations were generated, a drug in adhesive patch and a reservoir patch. When including the ethyl maltol zinc complex in the polyacrylate adhesive (Duro-TAK 9301) transdermal patch it did not deliver zinc into the skin. The existing commercial L-lysine/zinc transdermal patch was also shown not to deliver zinc into the skin. Using the reservoir patches 15 μg/cm² was delivered into the skin, but no zinc passed through the skin. The best reservoir patch delivered 55.3±20.6 μg/cm² into porcine skin, which would provide a 0.356 mg daily dose from a 1-inch patch placed on the skin for 24 h. The reservoir system provided a is significantly lower skin delivery compared to the solution, which delivered a daily dose of approximately 2 mg and thus further formulation optimisation is required.

### Example 2. Non-Limiting Iron Example

### 2.1 Materials

Iron chloride hexahydrate Cl3Fe·6H2O (99%) and sodium hexanoate (99%) were purchased from Acros Organic (UK). Ascorbic acid, ammonium acetate, glycerol, acetic acid and oleic acid were purchased from Fisher Scientific (UK). Concentrated hydrochloric acid, HCl (37%) was purchased from VWR Chemicals (UK). Ferrozine®, ethyl maltol, maltol, sodium propionate (≥99%), sodium oleate (≥95%), propylene glycol, PG (99%), diethylene glycol monethyl ether (Transcutol®) (99%), Kolliphore® EL, Tween 85, Span 20, ethyl acetate, ethanol (absolute ≥99.8%), methanol HPLC grade (≥99.9%), chloroform (≥99.8%), and n-hexane (≥95%) were purchased from Sigma Aldrich (UK). Phosphate buffered saline (Dulbecco A) (PBS) was purchased from Oxoid (UK). Kojic acid (99%) and polydimethylsiloxane (dimethicone) were purchased from Alfa Aesar (UK). Polyethylenglycol 400 (PEG 400) was purchased from Fluka. Triacetin was purchased from Merck (Germany). Liquid paraffin BP was purchased from Pinewood (UK). Natrsol M250 was donated from Ashland, Germany. Miglyol 810N was supplied from Azelis (UK). Cosmetic grade adhesives used included an acrylate copolymer (DURO-TAK 87-9301, 87-2516 and 87-2052, 87-2852, 87-2851) supplied by Henkel (USA), and an amine-compatible silicone (BIO-PSA 7-4302) supplied by Dupont (USA). Porcine ear skin was purchased from the local butcher Ginger Pig (UK).

### 2.2 Part 2. Iron Complex Manufacturing and Characterisation

### 2.2.1 Iron Complex Manufacturing

To form an iron 1:3 complex with the different complexing counterions the iron and the counterions were mixed in a molar ratio of 1:3.5 (Fe³⁺:counterion). To achieve this for the iron ethyl maltol, the iron maltol and the iron kojic acid complexes a 40 mM iron chloride hexahydrate solution was prepared by the addition of 219 mg to 20 mL water in a volumetric. A 100 mL stock solution of 46.7 mM counterion was prepared by weighing 654 mg, 588 mg, or 664 mg of ethyl maltol, maltol or kojic acid respectively, adding the solids to a 100 mL volumetric and solubilising in deionised water. The whole 100 ml of the iron chloride solution was mixed with 60 mL of the counterion solutions and the pH was adjusted to the required value (either pH 1.6, 4 or 7.4) using 1 M NaOH. The iron:counterion mixture was then split into 4-8 mL aliquots in glass vials of and placed in the freezer for a minimum of 24 h. The samples were then placed in a Lyotrap freeze dryer (LTE Scientific Ltd, Oldham, Greater Manchester, UK) with a freeze-drying temperature of 218±5 K. Once the drying chamber was in place and the draining valve closed, the internal pressure of the drying chamber was reduced to 0.03 mBar using a RV3 vacuum pump (Edwards Ltd, UK). The samples were freeze-dried for 2-3 days after which the vacuum was released and the generated powder was stored in the freezer until used.

Iron propionate was made by adding 1.08 g of iron chloride hexahydrate and 1.15 g of sodium propionate to 60 mL water. The pH of the complex solution was then either left unadjusted (pH 4) or adjusted to pH 7 using 1 M NaOH and split into vials and freeze-dried just as described above for the other complexes.

Iron hexanoate and iron oleate were produced in a 2-phase liquid extraction system. Iron chloride hexahydrate (1.08 g equivalent to 4 mM) and 1.7 g of sodium hexanoate (equivalent to 12 mM) were added to 60 mL water and pH adjusted to either 4 or 7 using 1 M NaOH. Then 80 mL ethanol and 140 mL n-hexane was added to the iron:counterion solution. In the case of iron oleate (pH remained unadjusted at pH 2.5), 1.08 g of iron chloride hexahydrate (equivalent to 4 mM) and 3.65 g of sodium oleate (equivalent 12 mM) were dissolved in 80 mL of ethanol, 60 mL distilled water was added and then 140 mL n-hexane was added. The two-phase mixtures were stirred for 4 h at 50° C. in a round bottom flask connected to a condenser over a stirring heating mantel. For both the hexanoate and oleate, the n-hexane layer was separated using a separation funnel and washed three times with water. The n-hexane was rotary evaporated under vacuum producing paste of iron hexanoate or iron oleate as previously descried in the literature [2]

### 2.2.2 Iron Complex Characterisation

2.2.2.1 Iron Quantification using UV Spectrophotometry

Ferrozine® was used to quantify iron due to its sensitivity and low interference with a range of biological matrices [3]. In the assay Ferrozine® reacts with the divalent iron to form a stable magenta complex in aqueous solution at pH 3-6 [3,4]. The method of the assay was developed by Carter 1971 [5]. The visible absorption spectrum of the ferrous complex of ferrozine exhibits a single sharp peak with maximum absorbance (λₘₐₓ) at 562 nm [3]. To conduct the assay a reducing agent was prepared by dissolving 20 mg ascorbic acid in a small quantity of water, to which 1.67 mL of concentrated hydrochloric acid was added and diluted to 100 mL. A 10% w/v ammonium acetate buffer was prepared, and its pH was reduced to 4-5 using acetic acid. A Ferrozine® solution was prepared by dissolving 60 mg of Ferrozine® in small volume of water containing 1 drop of concentrated HCl and made up to 20 mL with water. A 70.2 μg/mL elemental iron stock solution was prepared by weighing 27 mg of iron chloride hexahydrate (Cl₃Fe·6H₂O) and making it up to 100 mL with phosphate buffered saline. Standards were prepared in phosphate buffered saline via serial dilutions in the range 0-17.6 μg/mL elemental iron. A 0.5 mL of each of the standard solutions was mixed with 0.5 mL of the reducing agent solution and the mixture was allowed to stand at room temperature for 5 min. Then, 0.4 mL of the buffer solution was added and 0.1 mL of the Ferrozine® reagent were added prior to mixing. The absorbance of the final magenta-coloured complex, which develops within 5 min, was measured at 562 nm using a LAMBDA-35 (PerkinElmer, USA) spectrophotometer in 1 cm path length UV cell (quartz) and plate reader Spark (Tecan) using flat transparent 96-well plates (Thermo Scientific, UK Nunclon Delta surface). Standards in the range 0.4-7.0 μg/mL elemental iron were measured in the spectrophotometer while standards in the range 2.8-17.6 μg/mL elemental iron were measured using the plate.

### 2.2.2.2 Iron Complex Formation Confirmation Using UV Scanning Spectrophotometry

Solutions of iron chloride hexahydrate, the counterions and the complexes in methanol were prepared. A UV scan was obtained for them in the range of 320-800 nm using the Spark plate reader (Tecan, UK). An overlay of each complex with iron chloride hexahydrate and its corresponding counterion was plotted to assess a shift in the λₘₐₓ and hence to confirm the formation of the complex.

### 2.2.2.3 Infrared Spectroscopy

Complexes as well as counterions were placed on the sampling chamber of the FT-IR spectrometer (Perkin Elmer Frontier, UK) ensuring maximum contact with the crystal and secured with the contact arm. Measurements were taken between wavelengths (650-4,000 cm⁻¹), with 4 scans and a resolution of 4 cm⁻¹.

### 2.3 Part 2: Pre-Formulation Studies

### 2.3.1 Solubility Studies

Due to the high number of test samples (6 complexes plus the starting material, iron chloride hexahydrate) a visual solubility screen was used to screen the solubility in a range of solvents. Once a suitable candidate solvent or solvent mixture was identified, i.e., a solvent with >1 mg/ml was identified then exact solubility was quantitatively determined. Quantitative determination was performed by either incubating the complex in excess in the solvent using a shaking 37° C. water bath for 48 h or sonicating it for 2 hours at 40° C. or leaving overnight on a 50° C. hot plate. Once incubation period was completed, the exact solubility in a particular solvent was assayed (using the iron assay) after centrifugation (i.e. assay iron content in the supernatant using the Ferrozine® assay).

### 2.3.2 Iron Complex Solution Permeation Studies

The permeation of the iron complexes from the formulations into and across porcine skin mounted on Franz cells with a receiver fluid was tested (FIG. 4). This was based on the principles of the FDA, SUPAC-SS guidelines [6]. The developed colorimetric assay method was used for iron quantification.

### 2.3.2.1 Recovery of the Iron From the Skin

To verify the percentage of iron that could be recovered from the porcine skin after administration of the complexes, a 2.19±0.21 cm² piece of the skin was cut from a prepared piece of porcine ear. Tape stripping was used to isolate the stratum corneum (SC). This was achieved by placing a 300 g weight to press a piece of adhesive tape (Scotch 845 book tape, 3M, Bracknell, UK) to the surface of the skin for 10 sec. The tape was removed, and this separated one layer of the SC. This process was repeated until 20 layers of skin were taken. These strips were added into sample bottles. The epidermis was removed from the dermis physically using a scalpel and both the separated layers were cut into small pieces and placed into glass bottles. Test solutions of 885 mg/mL iron chloride hexahydrate in PG or 2.6 mg/mL iron ethyl maltol (complex made at pH 7.4) in PG were used to spike 10 μL or 100 μL respectively to each skin layer. This was incubated for 1 h, then 4 mL of extraction fluid (50:50 ethanol:PBS) was added to the stratum corneum vials and 2 mL to the epidermis and dermis vials. The stratum corneum samples were placed on vibrating plate for a minimum of 2 h and then vortexed prior assay. The epidermis and dermis samples were transferred to Eppendorf tubes, one stainless steel 5 mm bead was added then the tubes were loaded into the TissuLyser II (Qiagen, UK) for 15 min at 30 Hz. The samples were centrifuged (5 min at 13,000 rpm) and the supernatant was assayed for iron content. Note: the calibration solvent was 100% PBS and therefore a new calibration was drawn in 50:50 ethanol:PBS solution to quantify those samples.

### 2.3.2.2 Permeation and Deposition Studies

On the day of the experiment, the skin was defrosted. For the full thickness studies, the subcutaneous fat removed (2.5 mm thick) and for the dermatomes skin studies the tissued was dermatomed to a 1 mm thickness. The tissue was cut into the appropriate size sections (2.19±0.21 cm²) and mounted between donor and receiver compartments of Franz diffusion cells with the SC facing the donor compartment. Each cell was sealed using parafilm and 12 mm magnetic stir bars were placed into the receiver compartment. The receiver compartment was filled with the desired receiver solution and the Franz cells were placed in a stirring water bath at 37° C. Cells were allowed to equilibrate for 30 min after which the receiver fluid using was mixed using a syringe and the “zero hour” sample was withdrawn from the arm and compensate with the same volume of fresh pre-warmed solution. Following zero-hour sampling, the donor solution was placed in the donor compartment and covered with parafilm. Samples were withdrawn and compensated with fresh receiver solution at set time intervals for a 24 h duration. The iron in each sample was quantified using the method specified in Section 2.2.2.1. For the full thickness skin studies 50 mM saline (pH 7.4) was used as a receiver and the following donor solutions were assed: 1 mL of 1.4 g/mL iron chloride hexahydrate in PG, 1 mL of 2.7 mg/mL iron ethyl maltol in PG, 200 μL of 530 mg/mL iron oleate in 80:20 liquid paraffin:ethanol and 200 μL of 200 mg/mL iron oleate in Miglyol. There was no matrix interference noted in the assay. The dermatomed skin was used for the permeation of solutions of iron propionate (made at pH 4) and iron maltol (made at pH 4 and 1.6). An 80:20 PEG400:water receiver was used for iron propionate solutions, for which a complementary calibration (iron chloride in PEG400:water) was established for the assay of the samples. Iron propionate was dissolved in PEG400 (300 μL of 135 mg/mL) and iron propionate was dissolved in Transcutol© (300 μL of 92 mg/mL) due to the optimal solubility of the complexes in these vehicles. For iron maltol a 80:20 PG:water receiver was used along with a complementary calibration for iron assay (iron chloride in PG:water). The donor solution for the iron maltol made at pH 1.6 was 200 μL of 100 mg/mL complex in Transcutul®, for iron maltol made at pH 4 the donor solution was 200 μL of 23 mg/mL complex in PG and for iron maltol made at pH 4 the donor solution was 150 μL of 16 mg/mL complex in 7.5:1.5:1:1 water:PG:dimethicone:ethanol.

Once the permeation studies were completed the donor solutions were collected into bottles containing the extraction solution. This was followed by skin surface cleaning using four cotton buds, the first two were wetted by the extraction fluid and the second two were used dry. Then two tape striping procedures were performed using 2 tapes to remove any iron on the skin surface with the tape being added to the donor solution and cotton buds in the donor recover vial. For the full skin thickness skin, the Stratum corneum, the dermal and the epidermal layers were isolated in the same way as the recovery method (Section 2.3.2.1). Extraction fluid (50:50 PBS:ethanol) was then added to the tissues, 4 mL to the Stratum corneum, and 2 mL for each of the epidermis and dermis. For the dermatomed skin the whole skin section was chopped up and placed in a 2 mL extraction fluid (ethanol).

The Stratum corneum samples were placed on vibrating plate for a minimum of 2 h to extract the iron and then vortexed prior assay. The epidermis, dermis or dermatomed samples however were incubated with the extraction fluid for 1 h after which were transferred to Eppendorf tubes, one stainless steel 5 mm bead was added and the samples were loaded into the TissuLyser II (Qiagen, Germany) for 30-15 min at 30 Hz. The samples were then centrifuged (5 min at 13,000 rpm) and supernatant assayed.

### 2.4 Part 3 Patch Development

### 2.4.1 Adhesive Patch

### 2.4.1.1 Adhesive Patch Development

Pre-formulation studies were performed to determine the compatibility of the iron complexes with a range of transdermal patch adhesives (Table 15).

TABLE 45
Type of adhesives and their properties.

| Type of | Name of | | Solvent |
|---|---|---|---|
| adhesive | adhesive | Properties | Compatibility |
| Acrylates | DURO-TAK | No functional | Ethyl acetate |
| copolymer | 87-9301 | groups, 36.5% | Methanol |
| (pressure | (Henkel) | solids | |
| adhesives) | DURO-TAK | —OH functional | Ethyl acetate |
| | 87-2516 | group. Contains | |
| | (Henkel) | cross linker, | |
| | | 41.5% solids | |
| | DURO-TAK | —COOH | Ethyl acetate |
| | 87-2052 | functional | |
| | (Henkel) | group. Contains | |
| | | cross linker. | |
| | | Contains vinyl | |
| | | acetate, 47.5% | |
| | | solids | |
| | DURO-TAKO | —COOH | Ethyl acetate |
| | 87-2852 | functional | |
| | Henkel) | group. Contains | |
| | | cross linker, | |
| | | 33.5% solids | |
| | DURO-TAKO | —COOH | Ethyl acetate |
| | 87-2051 | functional | |
| | (Henkel) | group. Contains | |
| | | vinyl acetate, | |
| | | 51.5% solids | |
| Amine- | BIO-PSA | Low silicon | Propanol |
| Compatible | 7-4302 | content, 60% | |
| Silicone | (Dupont) | solid content | |
| | | in ethyl acetate | |

The compatibility of the complexes with the patch adhesives was assessed by mixing and visually checking the clarity of the mixture. The adhesives that did not dissolve the iron complexes with or without addition of co-solvents were eliminated from further investigations and initial formulations were designed from those remaining adhesives that facilitated acceptable iron complex solubility (>2 mg/ml).

### 2.4.1.2 Adhesive Patch Manufacturing

In the adhesives that were computable with the iron complexes the maximum drug loading of the complexes in the adhesive following solvent evaporation was determined. This was achieved by mixing the drug, adhesive and co-solvents until a clear solution was obtained at different drug:adhesive ratios and placing few drops of the mixture onto the patch backing sheet, leaving it to dry for 3-4 h, and examining the formed film on the surface of the backings for evidence of drug crystals.

The iron ethyl maltol, iron maltol and iron kojic complexes (all made at pH 7.4) showed compatibility with only one adhesive, DURO-TAK 9301. Following optimization of iron complex loading concentrations, a number of patches were made (Table 16).

TABLE 16
Adhesive patches compositions for iron ethyl maltol
(FeEM), iron maltol (FeM) and iron kojic (FeK).

| | | | | Theoretical |
|---|---|---|---|---|
| | | Per | Dry | patch |
| | | tray of | weight | composition |
| Patch | Ingredient | 100 cm² | equivalent | (%) |
| A1_FeEM | Iron ethyl | 33.7 mg | 33.7 mg | 4% |
| (4% | maltol | 2.25 mL | 810 mg | 96% |
| loading) | DT 9301 | 4.5 mL | | |
| | Ethyl | 3.37 mL | | |
| | acetate | | | |
| | Methanol | | | |
| A2_FeEM | Iron ethyl | 67.5 mg | 67.5 mg | 8.5% |
| (8.5% | maltol | 1.875 mL | 675 mg | 84% |
| loading) | DT 9301 | 60 μL | 60 mg | 7.5% |
| | PG | 3.75 mL | | |
| | Ethyl | 3.75 mL | | |
| | acetate | | | |
| | Methanol | | | |
| A3_FeM | Iron maltol | 67.5 mg | 67.5 mg | 8.5% |
| (8.5% | DT 9301 | 1.875 mL | 675 mg | 84% |
| loading) | PG | 60 μL | 60 mg | 7.5% |
| | Ethyl | 3.75 mL | | |
| | acetate | | | |
| | Methanol | 3.75 mL | | |
| A4_FeK | Iron Kojic | 67.5 mg | 67.5 mg | 8.5% |
| (8.5% | DT 9301 | 1.875 mL | 675 mg | 84% |
| loading) | PG | 60 μL | 60 mg | 7.5% |
| | Ethyl | 3.75 mL | | |
| | acetate | | | |
| | Methanol | 3.75 mL | | |

Adhesive patches were produced by mixing the required volume of adhesive and ethyl acetate then adding the complex dissolved in methanol. For most patches PG was added at the end of the process to act as a penetration enhancer when placed on the skin (Table 16). To produce the patch a sufficient volume of the formulation was applied to the patch backing to achieve a target coating of 8 mg/cm². The patch formulations were casted on a 10×10 cm square tray made of the backing (3M™ Scotchpak™ Polyester Backing Film Laminate 9730, UK). Following the complete drying of the solvents, the adhesive layer was covered by M3 Release liner (SCOTCHPAK 9755, UK), compressed, and then cut using 34 mm circular manual die cutter (SCP Super Cutting Press, UK).

### 2.4.1.3 Adhesive Patch Release Studies

To assess whether iron could be released from the adhesive' patches, a single patch was produced (Patch A1 from Table 16) and the release of iron was assessed using the Franz diffusion cells fitted with a regenerated cellulose membrane (RCM) (12-14 k molecular weight cut-off Medicell International UK). To prepare the RCM it was heated to 60° C. for 30 min after which the double layer of polymer (it is supplied as a dialysis membrane) was separated and glued onto the receiver chamber of the Franz cell and no donor chamber was used to allow the patch to be placed directly on the surface of the RCM. The receiver compartment was filled with 50:40:10 PG:methanol:water. The temperature of the Franz diffusion was maintained at 37° C. in a water bath. After 1 h equilibration at 37° C. in the water bath, 1 mL of the receiver fluid was taken (time point=0 min) and a fresh receiver solution was replaced to keep the volume content. The patch was then applied and a small weight (8.75 g) was applied to the top to ensure contact with the membrane throughout the 24 h study. A sample was taken after 24 h to assess release. The sample was assayed using the iron assay and a compatible calibration in the same medium (50:40:10 PG:methanol:water) was used.

### 2.4.1.4 Adhesive Patch Skin Permeation and Deposition Studies

To perform the skin permeation studies, dermatomed skin (1 mm) was glued onto the receiver compartment of the Franz cell that contained 80:20 PG:water. A zero hour sample was taken from the receiver fluid, the patches were placed on top of the skin and held in place by a small weight (8.75 g). The patches that were tested were A2, A3, A4 (Table 16) as well as the existing commercial iron transdermal patch. After 24 hours, the receiver fluid was assayed using the quantitative iron assay and a compatible calibration in the same medium (80:20 PG:water) was used. The skin deposition was determined by removing the dermatomed skin from the donor compartment when the 24 h was completed, chopping into smaller pieces using scissors and incubating with the extraction fluid, ethanol. The samples were then transferred to Eppendorf tubes containing one stainless steel 5 mm bead and loaded into the TissuLyser II (Qiagen, Germany) for 30 min 30 Hz. The samples were centrifuged (5 min at 13,000 rpm) and supernatant assayed for iron content.

### 2.4.2 Reservoir Patch

### 2.4.2.1 Reservoir Patch Development

Reservoir patches were development to increase the permeation into the skin after obtaining very little permeation from the adhesive patches. Iron complex was again screened with a range of suitable excipients and solvents to formulate a solution of high concentration that could be loaded into the patch reservoir. The solvents screened included ethanol, methanol, PG, PEG400, SDS, Tween 85, Span 20, Kolliphore®, Natrsol M250 and dimethicone (Note: methanol is not suitable for formulation but it was used to increase solubility in the other solvents. It was evaporated prior to formulation testing).

### 2.4.2.2 Reservoir Patch Manufacturing

The reservoir patch was made via heat sealing a packing material onto a controlled release membrane. To achieve this the patch backing (3M™ Scotchpak™ Polyester Backing Film Laminate 9730, UK) was placed on the surface of the heat press machine (HP230B, China), a 200 μL solution of complex was loaded onto the backing and then it was sealed using heat onto the controlled release membrane. A circular stainless-steel template (outer circle of 5.3 cm and inner circle of 3.2 cm) was used to form the seal at 90° C. for 1.5 min. The controlled release membranes that were used were M3 CoTran 9728, Whatman Nylon (0.45 μm nylon membrane, 47 mm diameter) and Millipore Durapore (0.22 μm GVHP membrane). A cellulose membrane (12-14 k molecular weight cut-off, Medicell International UK) was also tried, but it failed to achieve an efficient seal onto the patch backing. Table 17 summarises the reservoir patches that were made.

TABLE 17
Reservoir patch compositions.

| | | | Complex | |
|---|---|---|---|---|
| Patch | Complex | Solvent | Concentration | Membrane |
| R1 | Iron | PEG 400 | 135 mg/mL | M3 |
| | propionate | | | CoTran |
| | made at | | | |
| | pH 4 | | | |
| R2 | Iron | PG | 18 mg/mL | Nylon |
| | maltol | | | |
| | made at | | | |
| | pH 4 | | | |
| R3 | Iron | PG | 11 mg/mL | Durapore |
| | maltol | | | |
| | made at | | | |
| | pH 4 | | | |
| R4 | Iron | 7.5:1.5:1:1 | 16 mg/mL | Durapore |
| | maltol | water: PG: | | |
| | made at | dimethicone: | | |
| | pH 4 | ethanol | | |

### 2.4.2.3 Reservoir Patch Skin Permeation and Deposition

To perform the skin permeation studies dermatomed skin (1 mm) was glued onto the receiver compartment of the Franz cell that contained 80:20 PEG400:water for patch R1 or 80:20 PG water for patches R2-4 (Table 3). A zero hour sample was taken from the receiver fluid and the patches were placed on top of the skin and held in place by a small weight (8.75 g). After 24 h, the receiver was assayed for iron using the developed assay and a compatible calibration in the same medium (80:20 PEG400:water or PG:water) was used for the quantification. The skin deposition was determined by removing the dermatomed skin from the donor compartment when the 24 h experiment was completed, chopping it into smaller pieces and incubating with the extraction fluid, ethanol. The samples were then transferred to Eppendorf tubes containing one stainless steel 5 mm bead and loaded into the TissuLyser II (Qiagen, Germany) for 30 min at 30 Hz. The samples were then centrifuged (5 min at 13,000 rpm) and supernatant assayed for iron content.

### 2.5 Results

### 2.5.1 Part 1: Iron Complex Manufacturing and Characterisation

### 2.5.1.1 Iron Complex Manufacturing

The complexes had a range of percentage yields and a distinct appearance (Table 18).

TABLE 18
Summary of complexes percentage yield and appearance.

| | Percentage | |
|---|---|---|
| Complex | yield | Appearance |
| Iron ethyl maltol | Not | Orange |
| (made at pH 4) | calculated | powder |
| Iron ethyl maltol | 60% | Orange |
| (made at pH 7.4) | | powder |
| Iron maltol | Not | Orange |
| (made at pH 1.6) | calculated | powder |
| Iron maltol | Not | Orange |
| (made at pH 4) | calculated | powder |
| Iron maltol | 79% | Orange |
| (made at pH 7.4) | | powder |
| Iron kojic | Not | Dark orange |
| (made at pH 7.4) | calculated | powder |
| Iron propionate | 55% | Orange |
| (made at pH 4) | | powder |
| Iron propionate | 54% | Red/brown |
| (made at pH 7) | | flakes |
| Iron hexanoate | 48% | Red/brown |
| (made at pH 4) | | paste |
| Iron hexanoate | 44% | Red/brown |
| (made at pH 7) | | paste |
| Iron oleate | 89% | Red/brown |
| (made at pH 2.5) | | paste |

Under the assumption that the complexes formed a 1:3 iron:counterion complex then the molecular weight of iron ethyl maltol was 473 g/Mole, iron maltol 431 and iron kojic 479 g/Mole with 12-13% elemental iron. For the fatty acid complexes, the molecular weight of iron oleate was 898 g/Mole with only 6% elemental iron, 400 g/Mole for iron hexanoate with 14% elemental iron and 275 g/Mole for iron propionate with 20% elemental iron. FIG. 2 illustrates the structures of the complexes. As the pH of the solutions that formed the complexes deviated from physiological conditions it is likely that the assumption a pure 1:3 iron:counterion complex formed was unlikely to be 100% accurate as modify the pH increases the formation of the 1:2 and 1:1 complexes and this is important to consider when interpreting the data.

### 2.5.1.2 Iron Complex Characterisation

### Iron Quantification Using UV Spectrophotometry

A calibration curve was constructed using both a UV spectrophotometer and UV plate reader. The plate reader did detect concentrations <2.8 μg/mL of elemental iron and hence a higher concentration range than that used for spectrophotometer was used on the plate reader. Both techniques were verified as suitable to detect iron using the Ferrozine approach (Table 19).

TABLE 19
Iron assay validation.

| | ICH | | |
|---|---|---|---|
| Validation | level/ | Spectro- | Plate |
| parameter | limit | photometer | reader |
| Equation | | y = 0.1329x + | y = 0.1697x- |
| (μg/mL) | | 0.0397 | 0.0304 |
| Linearity | — | 0.4-7.0 | 2.8-17.6 |
| range | | | |
| (μg/mL) | | | |
| Linearity | >0.99 | 0.999 | 0.997 |
| (R², n = 3) | | | |
| LOD | — | 0.26 | 0.94 |
| (μg/mL) | | | |
| LOQ | — | 0.86 | 3.12 |
| (μg/mL) | | | |
| Accuracy | 95-105% | 97% (±2) | 96% (±4) |
| % (n = 3) | | | |
| Precision | | | |
| Intra-day | <2% | 7% (±5) | 7% (±3) |
| variability | | | |
| (% CV) | | | |
| Inter-day | <2% | 8% (±4) | 8% (±3) |
| variability | | | |
| (% CV) | | | |

### Linearity and Range

Both the spectrophotometer and the plate reader gave a calibration with a linearity within the ICH guidelines (R²>0.99). The spectrophotometer range was 0.4-7.00 μg/mL and the plate reader had a range of 2.8-17.6 μg/mL elemental iron.

The limits of detection (LOD) and limits of quantification (LOQ) were significantly different between the two detection methods. The spectrophotometer had a LOD and a LOQ of 0.26 and 0.86 μg/mL elemental iron respectively. While the plate reader had higher values of 0.94 and 3.12 μg/mL elemental iron respectively (Table 19).

### Precision

The assay precision was higher than the recommended ICH limit for both assays (<2%). However, it follows the predicted trend of being lower for higher concentrations and vice versa (Table 5). The reduction in the precision was thought to be a consequence of the multistep dilutions required for the assay. The increased variability compared to other assays was accounted for by performing a calibration curve every time the assay was used.

### Accuracy

The accuracy on the spectrophotometer was 97±2% while on the plate reader it was 96±4%. Both techniques were within the ICH acceptable range (95-105%).

Literature suggested that the assay sensitivity could be improved by allowing the colour to develop for 24 h at 20° C. or by increasing the reaction temperature to 37° C. for 135 min [7]. Therefore, the prolonged incubation and heating approaches were attempted to increase the sensitivity of the assay, however there was no advantage achieved in prolonging the analysis and hence a modified approach was not used.

Iron complex formation confirmation using UV scanning spectrophotometry

For iron complexed with ethyl maltol, maltol and kojic acid the lateral shift in the λₘₐₓ compared to the iron confirmed complex formation. All complexes were scanned at the same concentration. Iron ethyl maltol and iron maltol complexes were made at both pH 4 and 7.4 while iron kojic was only made at pH 7.4.

### Infrared Spectroscopy

Infrared (IR) spectroscopy analysis was conducted on all the iron:counterion complexes to confirm their formation. The spectra peaks for the counterions (ethyl maltol, maltol, kojic acid, sodium propionate, sodium hexanoate and sodium oleate) were obtained from literature and used to assign the peaks and thus interpret the IR spectra.

Iron ethyl maltol (Table 20) demonstrated the disappearance of the carbonyl bond (C═O) and the —OH group, which indicated the co-ordination between the iron ion and three ethyl maltol molecules and this confirmed the formation of the iron complex.

All the iron maltol (Table 21) complexes showed chemical shifts in the C—O that were indictive of complex formation regardless of the pH used to make the complexes

Iron kojic spectra (Table 22) showed several chemical shifts as well as the disappearance of the carbonyl group that were indictive of complex formation.

Iron propionate (Table 23) shifts in the carbonyl and hydroxy groups were indicative of complex formation when complex was made at pH 4, but these shifts were not seen at pH 7.

Iron hexanoate analysis showed the absence of shifts when complex was made at pH 4 (Table 24) which indicated no complexes were formed. While the shifts for the complex that was made at pH 7 indicate the formation of oligomers.

Iron oleate spectra (Table 25, FIG. 11) showed a shift in the carbonyl group indicating complex formation.

TABLE 20
Iron ethyl maltol IR summary.

| | | | Iron | Iron | |
|---|---|---|---|---|---|
| | | | ethyl | ethyl | Iron |
| | | | maltol | maltol | ethyl |
| | Refer- | | Batch 1 | Batch 2 | maltol |
| Wave- | ence | | (made | (made | (made |
| number | ethyl | Ethyl | at pH | at pH | at pH |
| (cm⁻¹) | maltol | maltol | 7.4) | 7.4) | 4) |
| 675-1000 | 843 | 830 | 709 | 711 | 830 |
| ═C₂—H | 939 | 935 | 831 | 832 | |
| ═C₃—H | 982 | 979 | 936 | 935 | |
| | | | 983 | 986 | |
| 1000-1300 | 1200 | 1186 | 1184 | 1185 | 1185 |
| C₃—O C₄—O | 1317 | 1259 | 1279 | 1279 | 1275 |
| C₅—O | | | | | |
| 1350-1480 | 1393 | 1390 | 1467 | 1470 | 1469 |
| C₆—H | | | | | |
| C₇—H | | | | | |
| 1400-1600 | 1559 | 1556 | 1506 | 1504 | 1507 |
| C₂═C₃ | | 1608 | 1565 | 1563 | 1565 |
| C₄═C₅ | | | 1603 | 1600 | 1581 |
| 1670-1820 | 1664 | 1644 | Dis- | | |
| C₁═O | | | appeared | | |
| 2850-3000 | | 2984 | Dis- | | |
| OH | | | appeared | | |

TABLE 21
Iron maltol IR summary.

| | | | Iron | Iron | Iron |
|---|---|---|---|---|---|
| | | | maltol | maltol | maltol |
| Wave- | Refer- | | (made | (made | (made |
| number | ence | | at pH | at pH | at pH |
| (cm⁻¹) | Maltol | Maltol | 7.4) | 4) | 1.6) |
| 675-1000 | 693 | 689 | 720 | 725 | 690 |
| ═C₁—H | | 849 | 850 | 831 | 834 |
| ═C₂—H | | 920 | 919 | 923 | 918 |
| 1000-1300 | 1212 | 1198 | 1041 | 995 | 997 |
| C₁—O | | 1257 | 1191 | 1192 | 1198 |
| C₄—O | | | | | |
| C₅—O | | | 1273 | 1255 | 1253 |
| 1420-1330 | 1334 | | | | |
| O—H | | | | | |
| 1350-1480 | 1372 | 1371 | 1461 | 1461 | 1460 |
| C₆—H | 1396 | 1462 | | | |
| 1400-1600 | 1645 | 1556 | 1500 | 1547 | 1554 |
| C₁═C₂ | | 1609 | 1566 | 1603 | 1607 |
| C₄═C₅ | | | 1602 | | |
| 3000-2840 | 3066 | 3065 | | 3072 | 3107 |
| 3200-2700 | 3288 | 3257 | | | |
| C₆—H | | | | | |

TABLE 22
Iron kojic (made at pH 7.4) IR summary.

| | Refer- | | | |
|---|---|---|---|---|
| Wave- | ence | | Iron | Iron |
| number | Kojic | Kojic | kojic | kojic |
| (cm⁻¹) | acid | acid | Batch 1 | Batch 2 |
| 675-1000 | 775 | 749 | 794 | 721 |
| ═C₁—H | 863 | 861 | 868 | 850 |
| ═C₄—H | 943 | 942 | 920 | 919 |
| 1000-1300 | 1074 | 1072 | 1024 | 1038 |
| C₁—O—C₅ | | 1140 | 1147 | 1197 |
| | | 1226 | 1277 | 1273 |
| 1350-1480 | 1472 | 1439 | 1459 | 1461 |
| C₆—H | | | | |
| 1400-1600 | 1611 | 1602 | 1503 | 1503 |
| C₁═C₂ | | | 1555 | 1564 |
| C₄═C₅ | | | 1610 | 1606 |
| 1670-1820 | 1660 | 1655 | Dis- | |
| C₃═O | | | appeared | |
| C₆—H | 2925 | 2920 | Dis- | |
| | 2854 | 2836 | appeared | |
| OH | 3270 | 3134 | 3415 | Dis- |
| | 3179 | | | appeared |

TABLE 23
Iron propionate IR summary

| | | | Iron | Iron | Iron | Iron |
|---|---|---|---|---|---|---|
| | | | propionate | propionate | propionate | propionate |
| | Reference | | Batch 1 | Batch 2 | Batch 1 | Batch 2 |
| Wavenumber | Sodium | Sodium | (made at | (made at | (made at | (made at |
| (cm⁻¹) | propionate | propionate | pH 4) | pH 4) | pH 7) | pH 7) |
| 675-1000 | 815 | 813 | 813 | 811 | 812 | 812 |
| —C₁—H | 880 | 878 | | | 879 | 878 |
| —C₂—H | | | | | | |
| 1000-1300 | 1298 | 1298 | 1082 | 1076 | 1078 | 1076 |
| C₃—O | | | 1303 | 1301 | 1297 | 1297 |
| 1350-1480 | 1370 | 1369 | | | 1369 | 1370 |
| —C₁—H | | | | | | |
| —C₂—H | | | | | | |
| 1420-1330 | 1417 | 1416 | 1426 | 1429 | 1418 | 1417 |
| O—H | | | | | | |
| 1465 | 1463 | 1462 | 1469 | 1465 | 1464 | 1461 |
| —C₁—H | | | | | | |
| —C₂—H | | | | | | |
| 1670-1820 | 1553 | 1557 | 1526 | 1515 | 1560 | 1556 |
| C₃═O | | | | | | |
| 2850-3000 | 2938 | 2971 | | | 2970 | 2974 |
| —C₁—H | 2972 | | | | | |
| —C₂—H | | | | | | |

TABLE 24
Iron hexanoate IR summary.

| | | Iron | Iron |
|---|---|---|---|
| Wave- | | hexanoate | hexanoate |
| number | Sodium | (made at | (made at |
| (cm⁻¹) | hexanoate | pH 4) | pH 7) |
| 675-1000 | 724 | 723 | |
| —C—H | 849 | 851 | |
| | 932 | 932 | |
| 1000-1300 | 1220 | 1222 | |
| C₆—O | | | |
| 1350-1480 | 1416 | 1413 | 1412 |
| C—H | 1445 | 1445 | |
| 1670-1820 | 1559 | 1559 | 1536 |
| C₆═O | | | |
| 2850-3000 | 2954 | 2958 | 2925 |
| C—H | | | |
| O—H | | | 3390 |

TABLE 25
Iron oleate IR summary.

| Wave- | Reference | | |
|---|---|---|---|
| number | Sodium | Sodium | Iron |
| (cm⁻¹) | oleate | oleate | oleate |
| 1420-1330 | 1396 | 1444 | 1406 |
| O—H | | | |
| 1670-1820 | 1643 | 1559 | 1712 |
| C═O | | | |
| 2850-3000 | 2856 | 2852 | 2856 |
| C—H | 2925 | 2921 | 2924 |
| | 3432 | | |

### 2.5.2 Part 2: Pre-Formulation Studies

### 2.5.2.1 Solubility Studies

The visual solubility of the iron complexes was assessed based on the amount of complex dissolved. However, once assayed, the solubility was quoted in terms of elemental iron. Iron chloride hexahydrate had a substantial solubility in PG representing 370 mg/mL elemental iron (1.4 g/mL of iron chloride hexahydrate). The solubility of iron ethyl maltol prepared at pH 7.4 (Table 26) was apparent in saline and PG, as well as in chloroform and methanol. An increase of complex solubility in PG and PEG400 was achieved when complex was made at pH 4.

There was also a noticeable difference in the solubility profile of iron maltol when it was made at different pH values. This could be a consequence of the difference in the hydrophobicity of the complexes made at different pH values as a result of different proportions of the 1:3, 1:2 and 1:1 complexes in the overall complexed system (Table 27).

The iron kojic solubility was screened and the results recorded (Table 28).

Iron propionate also showed a difference in solubility when it was made at different pH values (Table 29) with the complexes made at pH 7 shown to be insoluble in the tested solvents.

Iron hexanoate on the other hand showed a poor solubility (<20 mg/mL visual solubility of complex) when was made at pH 4 and 7 and tested in PG, PEG4000 and Transcutul.

Iron oleate was made without any pH adjustments (at pH 2.5) and had a substantial solubility in Miglyol 810N and liquid paraffin (Table 30).

TABLE 26
Iron ethyl maltol solubility screen
(*visual solubility).

| | Iron ethyl maltol | Iron ethyl maltol |
|---|---|---|
| Solvent | made at pH 7.4 | made at pH 4 |
| Saline | 360 μg/mL Iron | Not tested |
| | ethyl maltol | |
| | (44 μg/mL | |
| | elemental iron) | |
| PEG400 | <8 mg/mL Iron | 15 mg/mL Iron |
| | ethyl maltol* | ethyl maltol |
| | | (1.8 mg/mL |
| | | elemental iron) |
| PG | 2.7 mg/mL Iron | 8.3 mg/mL Iron |
| | ethyl maltol | ethyl maltol |
| | Equivalent to | Equivalent to |
| | 320 μg/mL | 1 mg/ml |
| | elemental iron | elemental iron |

| | |
|---|---|
| Glycerol | <8 mg/mL Iron ethyl maltol* |
| Transcutul | <8 mg/mL Iron ethyl maltol* |
| Triaceten | <8 mg/mL Iron ethyl maltol* |
| Liquid | <8 mg/mL Iron ethyl maltol* |
| Paraffin | |
| Miglyol 810N | <8 mg/mL Iron ethyl maltol* |
| Oleic acid | <8 mg/mL Iron ethyl maltol* |
| Ethyl acetate | <8 mg/mL Iron ethyl maltol* |
| Methanol | ≥20 mg/mL Iron ethyl maltol* |
| Hexane | <8 mg/mL Iron ethyl maltol* |
| Chloroform | ≥20 mg/mL Iron ethyl maltol* |

TABLE 27
Iron maltol solubility screen
(*visual solubility).

| | Iron | Iron | Iron |
|---|---|---|---|
| | maltol | maltol | maltol |
| | made at | made at | made at |
| Solvent | pH 7.4 | pH 4 | pH 1.6 |
| PEG400 | <20 mg/ | 20 mg/ | <6 mg/ |
| | mL Iron | mL Iron | mL Iron |
| | maltol* | maltol | maltol* |
| | | Equivalent | |
| | | to 2.7 mg/ | |
| | | mL ele- | |
| | | mental iron | |
| PG | ≥20 mg/ | 17 ± 5 mg/ | <6 mg/ |
| | mL Iron | mL Iron | mL Iron |
| | maltol* | maltol | maltol* |
| | | Equivalent | |
| | | to 2.3 ± 0.6 | |
| | | mg/mL ele- | |
| | | mental iron | |
| Transcutul | <20 mg/ | <6 mg/mL | 100 mg/ |
| | mL Iron | Iron maltol* | mL Iron |
| | maltol* | | maltol |
| | | | Equivalent |
| | | | to 13 mg/ |
| | | | mL ele- |
| | | | mental iron |

| | |
|---|---|
| Triaceten | <6 mg/mL Iron maltol* |
| Oleic acid | <6 mg/mL Iron maltol* |
| Ethyl | <6 mg/mL Iron maltol* |
| acetate | |
| Ethanol | ≥6 mg/mL Iron maltol* |
| Methanol | ≥18 mg/mL Iron |
| | maltol* |
| Propanol | <6 mg/mL Iron maltol* |
| Chloroform | ≥20 mg/mL Iron |
| | maltol* |
| n-Hexane | <6 mg/mL Iron maltol* |

TABLE 28
Iron kojic (made at pH 7.4) solubility screen
(*visual solubility).

| Solvent | Solubility |
|---|---|
| PEG400 | <6 mg/mL Iron kojic* |
| Glycerol | <6 mg/mL Iron kojic* |
| PG | ≥8 mg/mL Iron kojic* |
| Transcutul | ≥ 8 mg/mL Iron kojic* |
| Triaceten | <6 mg/mL Iron kojic* |
| Oleic acid | <6 mg/mL Iron kojic* |
| Ethyl acetate | <6 mg/mL Iron kojic* |
| Methanol | ≥18 mg/mL Iron kojic* |
| Propanol | <6 mg/mL Iron kojic* |
| Chloroform | <6 mg/mL Iron kojic* |
| Hexane | <6 mg/mL Iron kojic* |

TABLE 29
Iron propionate solubility screen
(*visual solubility).

| | Iron | Iron |
|---|---|---|
| | propionate | propionate |
| | made at | made at |
| Solvent | PH 7 | pH 4 |
| PEG400 | <6 mg/mL | 135 mg/ |
| | Iron | mL Iron |
| | propionate* | propionate |
| | | Equivalent |
| | | to 27 mg/ml |
| | | elemental |
| | | iron |
| Glycerol | Not tested | <6 mg/mL |
| | | Iron |
| | | propionate* |
| PG | <6 mg/mL | <6 mg/mL |
| | Iron | Iron |
| | propionate* | propionate* |
| Transcutul | <6 mg/mL | 92 mg/mL |
| | Iron | Iron |
| | propionate* | propionate |
| | | Equivalent |
| | | to 18.5 |
| | | mg/mL |
| | | elemental |
| | | iron |
| Triaceten | <6 mg/mL | <6 mg/mL |
| | Iron | Iron |
| | propionate* | propionate* |
| Liquid | Not tested | <6 mg/mL |
| Paraffin | | Iron |
| | | propionate* |
| Miglyol | Not tested | <6 mg/mL |
| | | Iron |
| | | propionate* |
| Ethyl | <6 mg/mL | <6 mg/mL |
| acetate | Iron | Iron |
| | propionate* | propionate* |
| Ethanol | <6 mg/mL | Not tested |
| | Iron | |
| | propionate* | |
| Methanol | <6 mg/mL | Not tested |
| | Iron | |
| | propionate | |
| Hexane | <6 mg/mL | <6 mg/mL |
| | Iron | Iron |
| | propionate* | propionate* |

TABLE 30
Iron oleate solubility screen
(*visual solubility)

| Solvent | Solubility |
|---|---|
| PEG400 | <8 mg/mL Iron oleate* |
| Glycerol | <8 mg/mL Iron oleate* |
| PG | <8 mg/mL Iron oleate* |
| Transcutul | <8 mg/mL Iron oleate* |
| Triaceten | <8 mg/mL Iron oleate* |
| Liquid | 680 mg/mL Iron oleate |
| Paraffin | Equivalent to 41 mg/mL elemental iron |
| Miglyol | 200 mg/mL Iron oleate |
| 810N | Equivalent to 12.5 mg/mL elemental iron |

Recovery of Iron from the Skin

The percentage of iron chloride hexahydrate and iron ethyl maltol (in PG) recovered from skin was determined through the extraction study (Table 31). The measured drug extraction was within the required 100±15% recovery for iron ethyl maltol from the Stratum corneum and dermis, and slightly lower for the epidermis. However, for iron chloride hexahydrate it was not. Attempts were taken to improve this recovery via the use of different extraction fluid (e.g. pyridine-2,6-dicarboxylic acid, PDCA) as well as the use of nitric acid [8]. However, this did not improve the recovery and since in the permeation studies iron chloride in PG was not a suitable candidate no further action was taken.

TABLE 31
Recovery of iron chloride and iron ethyl maltol from
porcine skin layers. Data represents the mean ± one
standard deviation (n = 3).

| Compound | Recovery in skin (%) | |
|---|---|---|
| Iron chloride | Stratum corneum | 155 ± 7 |
| | Epidermis | 51 ± 6 |
| | Dermis | 59 ± 17 |
| Iron ethyl maltol | Stratum corneum | 104 ± 25 |
| | Epidermis | 78 ± 10 |
| | Dermis | 96 ± 8 |

### Permeation and Deposition Studies

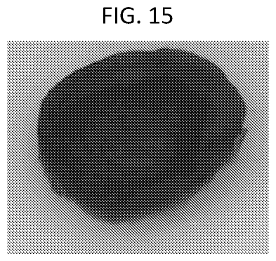

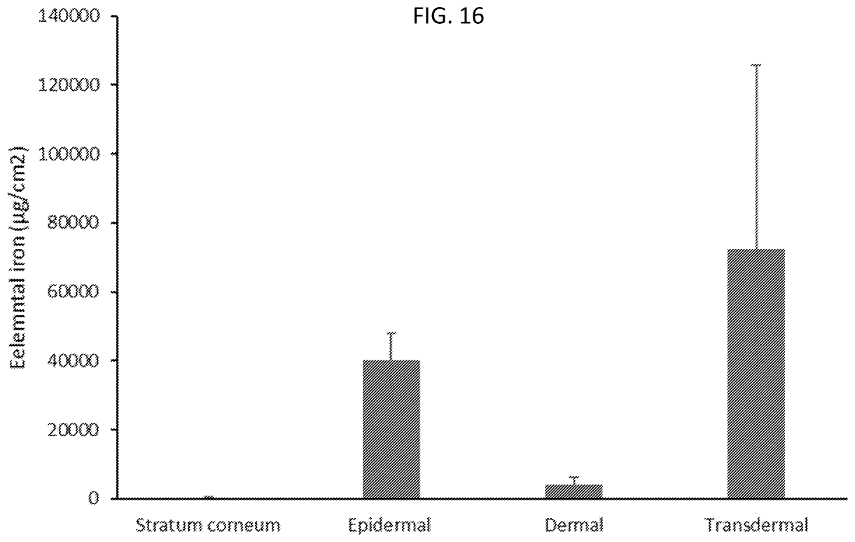

Iron chloride hexahydrate had a high solubility in PG allowing the dosing of 370 mg elemental iron, of which 180 mg permeated (48%) the skin after 24 h through the 2 cm² permeation area of the experiment. While that dose was 40 times above the desired target (4.6 mg), it led to the necrosis of the skin (FIG. 15), making it inappropriate candidate for skin delivery. The amount permeated and deposited in each skin layer is illustrated in FIG. 16.

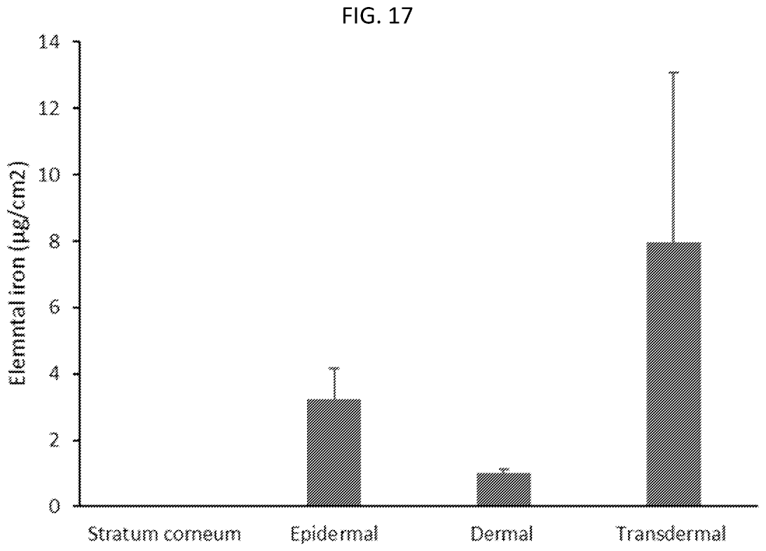

Iron ethyl maltol did not cause any visible skin damage, however the amount that deposited and permeated the tissue was much lower (FIG. 17), resulting in a total delivered dose of 12 μg/cm².

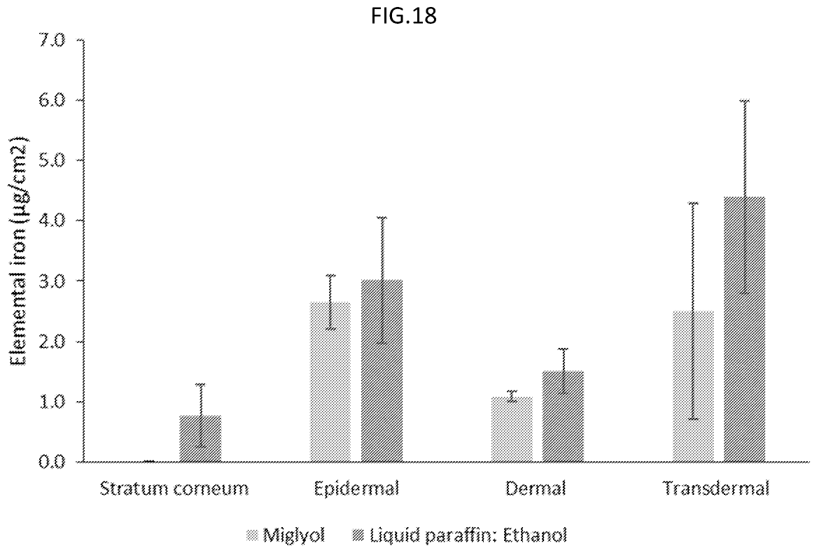

For iron oleate, 2.5±1.8 and 4.4±1.5 μg/cm² elemental iron permeated the skin over 24 h when Miglyol and liquid paraffin:ethanol were used as the donor solutions respectively. The deposition was 3.7 μg/cm² and 5.3 μg/cm², for all the layers combined, respectively (FIG. 18). There was no observed skin damage with both formulations.

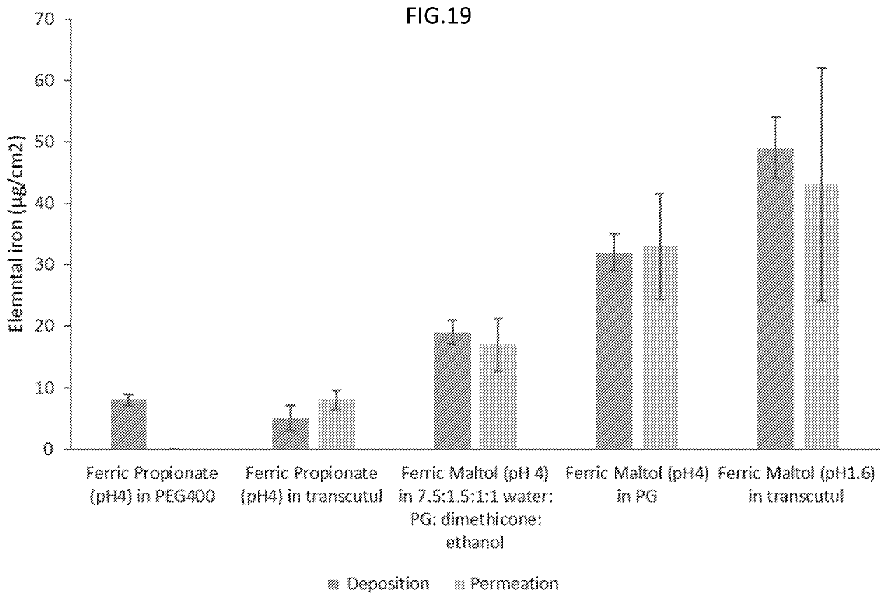

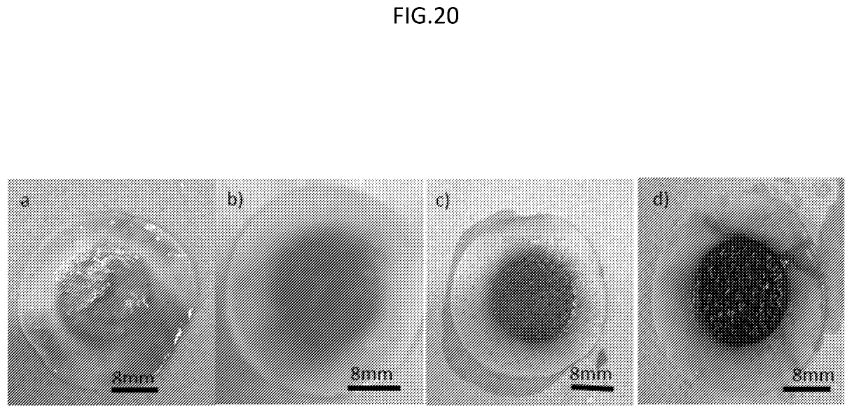

Solutions of iron propionate (made at pH 4) and iron maltol (made at pH 4 and 1.6) permeated and deposited in the skin (FIG. 19). The transdermal delivery of iron maltol wad significantly lower pH 4 compared to the same complex mad at pH 1.6 (t-test p value 0.15). However, the iron maltol made at pH 1.6 caused skin necrosis (FIG. 20). Therefore, the iron maltol made at pH 4 and dissolved in PG was considered the best solution with a total delivered dose of 65 μg/cm².

### 2.5.3 Part 3: Patch Development

### 2.5.3.1 Adhesive Patch

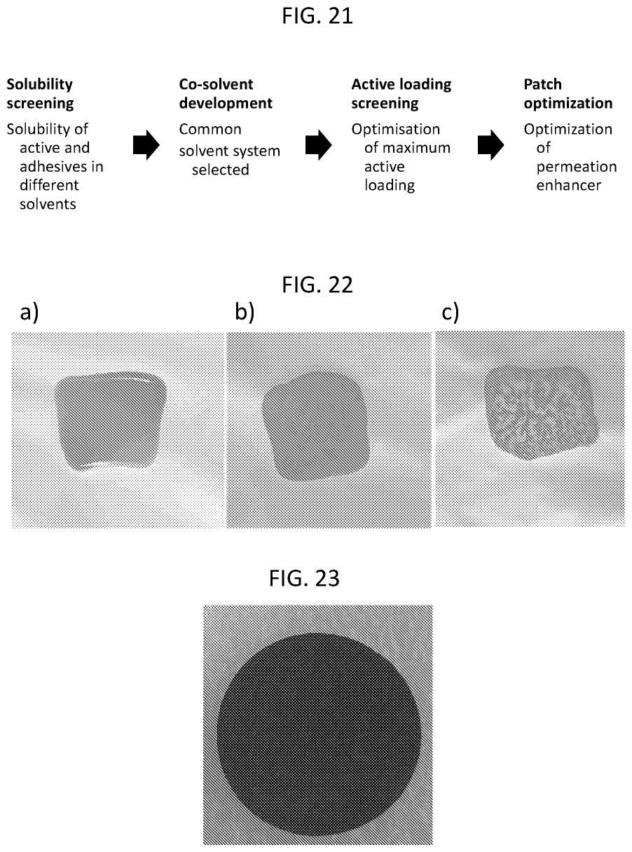

The patch development process was performed using a systematic process (FIG. 21). Initially a common solvent for both drug and polymers was identified. This was followed by optimising drug loading, adhesive properties, adhesive layer thickness, and inclusion/percentage of permeation enhancers.

### Adhesive Patch Development and Manufacturing

Iron ethyl maltol was only compatible with DURO-TAK 9301. The concentration of the stock solution of iron ethyl maltol in methanol had a major influence on the complex loading achieved in the adhesive patch. For example, initially a 4% loading was not achieved when the stock concentration was 20 mg/mL however when it was reduced to 10 mg/mL a 4% loading was achieved (Table 32).

TABLE 32
Iron ethyl maltol (FeEM) patch screen.

| | | Duro- | | |
|---|---|---|---|---|
| | | TAK | | |
| Drug | | 9301 | | |
| loading | FeEM | Adhesive | Permeation | |
| (%) | (mg) | (mg) | enhancer | Solubility |
| 2.5-10% | 1.25- 5 | 45 | — | Precipitated |
| 4% | 1.87 | 45 | — | Clear |
| 4% | 2 | 45 | 2 mg PG | Clear |
| 7.5-10% | 3.75- 5.5 | 45 | 4-5.5 mg PG | Precipitated |
| 5% | 2.5 | 45 | 2.5 mg | Precipitated |
| | | | Transcutol | |
| 5% | 2.5 | 45 | 5 mg | Clear |
| | | | Transcutol | |
| 7.5% | 4 | 45 | 4 mg | Precipitated |
| | | | Transcutol | |
| 4% | 2 | 45 | 2 mg | Clear |
| | | | PEG400 | |
| 5% | 2.6 | 45 | 2.6 mg | Precipitated |
| | | | PEG400 | |
| 5% | 2.5 | 45 | 5 mg | Precipitated |
| | | | PEG400 | |
| 6% | 3.3 | 45 | 3.3 mg | Precipitated |
| | | | PEG400 | |
| 10-7.5% | 5.5-4 | 45 | 5.5-4 mg | Precipitated |
| | | | PEG400 | |

The compatibility/solubility of the adhesive with solvents and the iron complexes was established by mixing at different drug:adhesive ratios and placing a drop of solution onto the patch backing sheet, leaving the slide to dry for 3-4 h, and examining the formed film on the surface of the backings for evidence of drug crystals (FIG. 22). The formulation that did not show precipitation was then cast on a 10×10 cm square using the backing. Following complete drying of the solvents, the adhesive layer was covered by a release liner, compressed, and then cut using 34 mm circular manual die cutter (FIG. 23).

Using a 10% loading of iron ethyl maltol with DURO-TAK 9301 a range of solubility and permeation enhancers were used to try and increase the iron complex solubility including: ethyl butyrate, PVP, PVP K90, Kolliphore, Natrosol 250L, Lutrol F127, Lutrol F68, Lutrol micro 68M, triethyl citrate, PEG 6000, Compritol, glycerol, xylitol and DSMO. However, they did not enhance the iron complex solubility in the adhesive. Consequently, a patch containing 8.5% complex was made using the stock solution of iron ethyl maltol in methanol (18 mg/mL) to manufacture Patch A2 (Table 16).

Iron maltol and iron kojic complexes were also only compatible with DURO-TAK 9301. Similarly, their solubility screening studies (Tables 33 and 34) showed a maximum loading of 4% when the stock solution was 10 mg/mL and patches A3 and A4 (Table 16) were optimised with 18 mg/mL stock resulting in 8.5% loading.

TABLE 33
Iron maltol (FeM) patch screen.

| | | 9301 | | |
|---|---|---|---|---|
| Drug | FeM | Adhesive | Permeation | |
| loading | (mg) | (mg) | enhancer | Solubility |
| 10% | 5.5 | 45 | — | Precipitated |
| 10% | 5.5 | 45 | 5.5 mg PG | Precipitated |
| 10% | 5.5 | 45 | 5.5 mg Transcutol | Precipitated |
| 8% | 4 | 45 | — | Precipitated |
| 7.5% | 4 | 45 | 4 mg PG | Precipitated |
| 7% | 4 | 45 | 8 mg Maltol | Precipitated |
| 6% | 2.8 | 45 | — | Precipitated |
| 4% | 2 | 45 | — | Clear |
| 4% | 2 | 45 | 2 mg PG | Clear |

TABLE 34
Iron kojic (FeK) patch screen.

| | | 9301 | | |
|---|---|---|---|---|
| Drug | FeK | Adhesive | Permeation | |
| loading | (mg) | (mg) | enhancer | Solubility |
| 10% | 5.5 | 45 | — | Precipitated |
| 10% | 5.5 | 45 | 5.5 mg PG | Precipitated |
| 10% | 5.5 | 45 | 5.5 mg Transcutol | Precipitated |
| 8% | 4 | 45 | — | Precipitated |
| 7.5% | 4 | 45 | 4 mg PG | Precipitated |
| 7% | 4 | 45 | 8 mg Maltol | Precipitated |
| 4% | 2 | 45 | — | Clear |
| 4% | 2 | 45 | 2 mg PG | Clear |

### Adhesive Patch Release Studies

Patch A1 (Table 16) was used for to test the iron complex release (4% loading patch of an 8 mg/cm² iron ethyl maltol). Therefore, per the 2 cm² permeation area of Franz cell, there was 16 mg of formulation applied of which only 4% was the iron complex i.e. 640 μg, of which only 12% was elemental iron (77 μg). After 24 h, 28 μg of elemental iron was released from the patch accounting for 37% of the patch payload.

### Adhesive Patch Skin Permeation and Deposition

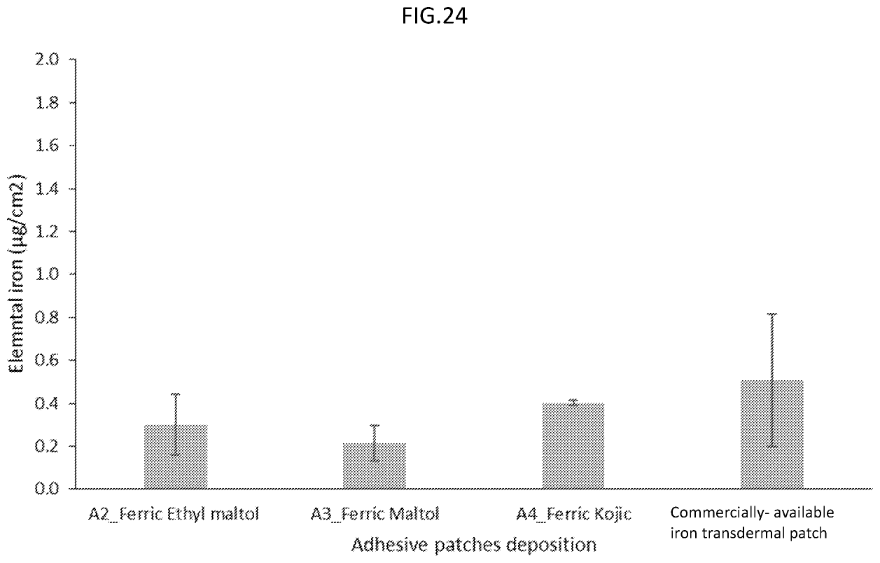

Patches A2, A3, A4 (Table 16) and an existing commercial transdermal iron patch were tested for skin permeation and deposition. All four patches showed zero iron permeation through the skin with iron deposition amounting to an average of 0.3 μg/cm² elemental iron (FIG. 24). There was no significant difference between the deposition of an existing commercial iron transdermal patch and our highest adhesive, iron kojic, deposition (t-test p value, 0.5). Consequently, it was concluded that a simple adhesive patch design was problematic for the delivery of iron and as an alternative a reservoir patch was designed and tested.

### 2.5.3.2 Reservoir Patch

### Reservoir Patch Development and Manufacturing

In order to maximise the iron permeation from the reservoir patch, the concentration of iron complex in the reservoir co-solvents was maximised. The visual solubility of iron maltol (made at pH 7.4) was screened with Span 80, SDS, Tween 80 and Kolliphore© EL (Table 35), however, it did not exceed 32 mg/mL. A solubility screening exercise was therefore conducted for iron maltol made at a pH 4 (Tables 36 and 37).

Initial solubility screening with dimethicone looked promising (Table 37), however it was noted that upon standing the samples precipitated (F1-F3) and when scaled up (F4 and F5) there was a significant reduction in solubility. F4 was the only formulation suitable for use in a permeation study (as solution and as Durapore patch, R4).

TABLE 35
Iron maltol made at pH 7.4 (*visual solubility).

| Complex | | |
|---|---|---|
| (mg) | Excipients | Observation |
| 40 | 12 mg SDS | Precipitated |
| | 0.125 mL ethanol | |
| | 0.5 mL PG | |
| 30 | 6 μL Tween 85 | Precipitated |
| | 0.125 mL ethanol | |
| | 0.5 mL PG | |
| 20 | 6 μL Span 20 | Precipitated |
| | 0.125 mL ethanol | |
| | 0.5 mL PG | |
| 10 | 62.5 μL PG | Precipitated |
| | 62.5 μL water | |
| | 125 μL Kolliphore | |
| 10 | 125 μL PG | Clear |
| | 60 μL ethanol | (32 mg/mL |
| | 62.5 μL water | complex*) |
| | 62.5 μL Kolliphore | |
| 10 | 62.5 μL PG | Clear |
| | 60 μL ethanol | (32 mg/mL |
| | 125 μL water | complex* |
| | 62.5 μL Kolliphore | |

TABLE 36
Iron maltol made at pH 4.

| Complex | | |
|---|---|---|
| (mg) | Excipients | Observation |
| 100 | 2 mL methanol | Precipitate |
| | 1 mL 80:20 | upon methanol |
| | PEG400:PEG1000 | evaporation |
| 20 | 0.2 mL Kolliphore | Precipitated |
| 20 | 0.1 mL Kolliphore | Precipitated |
| | 0.1 mL PEG400 | |
| 20 | 0.1 mL Kolliphore | Precipitated |
| | 0.1 mL PEG400 | |
| | 10% SDS | |
| 10 | 50 μL 2% Nitrosol | 22 mg/mL |
| | 50 μL PG | complex |

TABLE 37
Iron maltol (made at pH 4) screen with dimethicone.

| | Complex | Water | Dimethicone | PG | Ethanol | |
|---|---|---|---|---|---|---|
| Formulation | (mg) | (μL) | (μL) | (μL) | (μL) | Solubility |
| F1 | 10 | 75 | 25 μl | 20 μl | | 19 mg/mL complex |
| | | | | | | (2.5 mg/mL |
| | | | | | | elemental iron) |
| F2 | 15 | 75 | 10 | 15 | 10 | 128 mg/mL complex |
| | | | | | | (15.5 mg/mL |
| | | | | | | elemental iron) |
| F3 | 10 | 75 | 10 | 15 | 10 | 76 ± 17 mg/mL |
| | | | | | | complex |
| | | | | | | (9 ± 1.5 mg/mL |
| | | | | | | elemental iron) |
| F4 (F2 × 20) | 290 | 1500 | 200 | 300 | 200 | 16 mg/mL complex |
| | | | | | | (2 mg/mL elemental |
| | | | | | | iron) |
| F5 (F3 × 5) | 50 | 375 | 50 | 75 | 50 | 18 mg/mL complex |
| | | | | | | (2.4 mg/mL |
| | | | | | | elemental iron) |

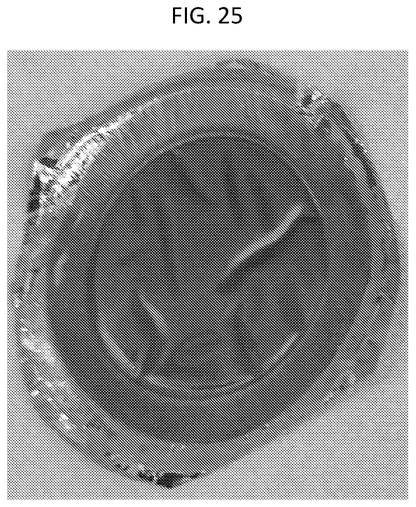

Four reservoir patches were made using complexes made at pH 4 (FIG. 25). R1 was made with iron propionate in PEG400 (135 mg/mL complex using a CoTran release controlling membrane). While patches R2, R3 and R4 were made using iron maltol. R2 included 18 mg/mL iron maltol complex in PG using a nylon rate controlling membrane, R3 included 11 mg/mL iron maltol in PG using a Durapore membrane and R4 included 16 mg/mL iron maltol in 7.5:1.5:1:1 water:PG:dimethicone using a Durapore membrane.

### Reservoir Patch Skin Permeation and Deposition

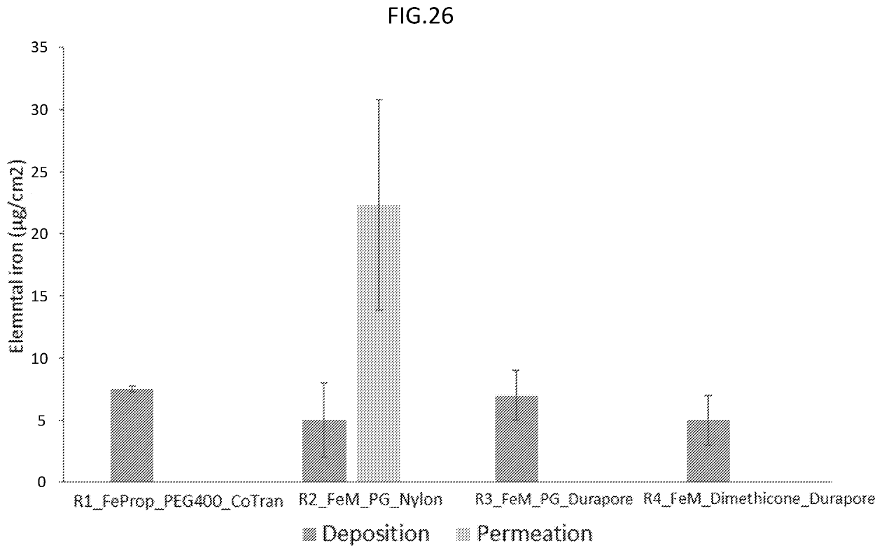

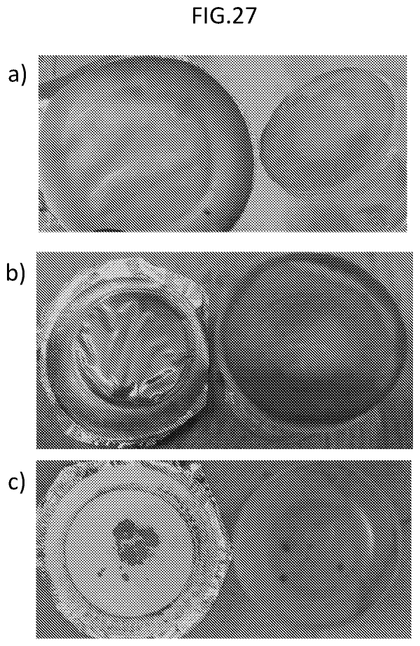

The four reservoir patches showed better skin permeation and deposition compared to the previously assessed adhesive patches (FIG. 26). The ferric propionate reservoir patch formulated using a PG solution gave approximately the same deposition into the skin compared to the same complex applied to the skin as a PG solution (8 μg/cm²) (FIG. 19 vs. FIG. 26), but neither the patch nor the solution allowed iron to pass through the skin. The two iron maltol patches that used the Durapore rate controlling membrane showed similar delivery of iron into the skin as the iron propionate patch (5±2 μg/cm² for the dimethacone patch and 7±2 μg/cm² for the iron maltol patch. The iron maltol durapore patches gave a significantly lower deposition into the skin compared to the previously tested solutions applied directly to the skin (see FIG. 19) and they did not show any transdermal penetration. The iron maltol patch using the nylon membrane gave the best penetration into the skin at 5±3 μg/cm² and the best transdermal penetration at 22±8 μg:cm². Despite the good permeation of the complex into and through the skin using the iron maltol, images of the skin after the application of patch show that the patch did not apply the solution to the skin evenly suggesting that there is scope for further patch development and potential to improve the delivery further. When compared to an existing commercial iron transdermal adhesive patch, the iron maltol reservoir system delivered 53 times more iron into the skin (iron maltol total delivery was 27 μg/cm² vs the existing commercial iron transdermal adhesive patch 0.51 μg/cm²).

### 2.6 Conclusions

Four different iron complexes (iron ethyl maltol, iron maltol, iron propionate and iron oleate) were manufactured, and their formation was confirmed using IR and UV spectroscopy. Interestingly, the solubility and skin compatibility of the formed complex was dependent on the pH of complex solution prior to lyophilisation, which is a novel finding that may make the patches patentable. The iron maltol complex produced at pH 4 demonstrated a high solubility (18 mg/mL in PG and PEG400) without inducing skin necrosis. Hence, it was selected for further development. Two types of product prototypes were manufactured: a drug-in-adhesive and a reservoir patch. However, the iron complexes in the polyacrylate adhesive (Duro-TAK 9301) transdermal patch did not delivery clinically meaningful quantities of iron into the skin. Similarly, the existing commercial iron transdermal adhesive patch showed limited skin delivery (4.9 μg of elemental iron/3.1×3.1 patch) and no permeation. A novel solution of iron maltol (made at pH 4) in PG (18 mg/mL), formulated into a reservoir patch prototype with the aid of a nylon membrane was the best system assessed in this work. If the patch was scaled up to a 3.1 cm×3.1 cm reservoir patch then based on its delivery in this study per cm² of skin it would deliver 360 μg of iron maltol into porcine skin and 1650 μg through porcine skin over 24 hours, which would provide a 2 mg of iron maltol over 24 hours from 3.1×3.1 cm patch. However, images of the reservoir patch and skin after delivery demonstrate that the contact between the patch and the skin could be optimised further and the consequences of the orange colour of the complex needs to be investigated.

### Example 3. Non-Limiting Vitamin D Example

### 3.1 Materials

Cholecalciferol (≥98%), phosphorus (V) oxychloride (POCl₃) (≥99.0 0 anhydrous tetrahydrofuran (THF) (≥99.9%), triethylamine, magnesium sulphate (MgSO₄), acetonitrile (≥99.9%), deuterated methanol (methanol-d₄) and propylene glycol (PG) (≥99.5%) were purchased from Sigma Aldrich, UK. n-Hexane fractions, ethyl acetate, sodium hydroxide (NaOH), hydrochloric acid (HCl), absolute ethanol and trifluoroacetic acid (TFA) HPLC grade (≥99%) were purchased from Fisher Scientific Ltd UK. Deuterated chloroform (CDCl₃) (99.8%, 0.03% TMS) was purchased from Merck, Germany. Phosphate buffered saline (Dulbecco A) (PBS) was purchased from Oxoid, and formic acid was purchased from Honeywell. A range of cosmetic grade adhesives were used, polyisobutene (Oppanol B12 and Oppanol N100) were supplied by BASF (Germany), Acrylates copolymer (DURO-TAK 87-9301, 87-2516 and 87-2052) were purchased from Henkel (USA), and amine-Compatible Silicone (BIO-PSA 7-4302) were purchased form Dupont (USA). Porcine ear skin was purchased from the local butcher Ginger Pig, UK. All chemicals were reagent grade unless otherwise stated.

### 3.2 Part 1: Phosphorylated Vitamin Synthesis and Characterisation

### 3.2.1 Cholecalciferol Phosphate Disodium Synthesis

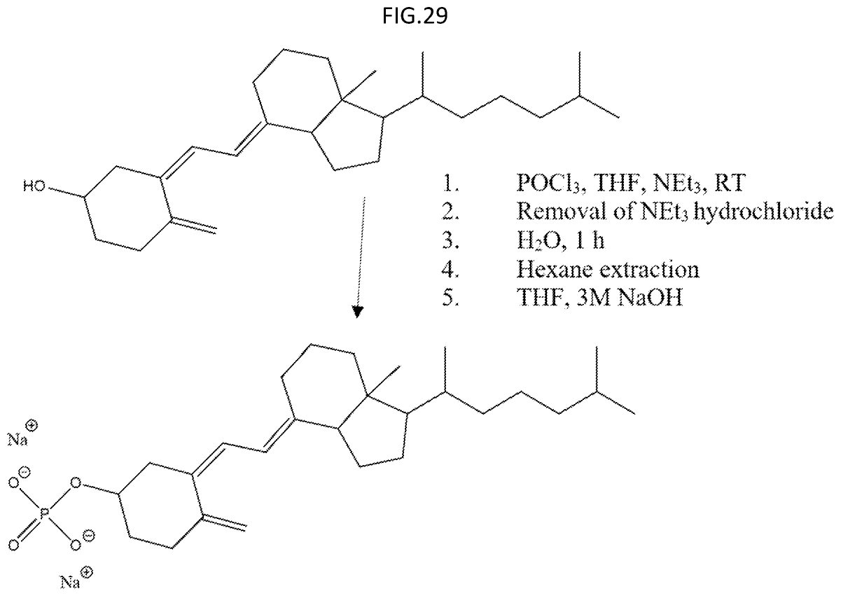

The synthesis of cholecalciferol phosphate was performed in two steps, a nucleophilic attack by phosphorous oxychloride (POCl₃) in the presence of triethylamine, followed by the hydrolysis of the oxychloride groups. Cholecalciferol (1.50 g, 3.90 mmol) was dissolved in anhydrous THF (25 mL) and triethylamine (1.63 mL, 11.7 mmol, 3 eq) and placed under nitrogen. Maintaining the nitrogen conditions, the vit-D:THF solution was added drop-wise to a POCl₃ solution in THF (POCl₃ (0.510 mL, 5.46 mmol, 1.4 eq) over a period of 40 min, (15 mL). This reaction was allowed to stir at room temperature under nitrogen for 2.5 h to generate the intermediate, phosphorochloridate. Thin layer chromatography (TLC) analysis was performed (6:4, hexane:ethyl acetate) using Merck TLC aluminium sheets silica 60-F254 to confirm the reaction was complete. The main reaction by-product, solid white triethylamine hydrochloride powder, was removed by suction filtration using a Buchner filter and 1 μm Whatman filter paper. Distilled water (7 mL) was added to the phosphorodichloridate to exchange the chloride groups with hydrogens to make cholecalciferol phosphate, this solution stirred for 1 h. The product was then extracted from the solution by double extraction, firstly using n-hexane (150 mL), with the organic layer being dried over by anhydrous MgSO₄. A yellow oil obtained after rotary evaporation of the hexane was then dissolved in TFH (15 mL) and 3M NaOH (5 mL) stirring for few minutes. The cholecalciferol phosphate sodium salt preferentially partitioned into the aqueous layer and hence the THF layer was removed, and absolute ethanol was then added to the aqueous layer and it was boiled, resulting in white/yellow precipitate (FIG. 29). The synthetic process were repeated three times to generate 3 batches of vitamin D phosphate disodium material (n=3).

### 3.2.2 Cholecalciferol Phosphate Acid Synthesis

The approach taken to generate the acidic form of the phosphorylated vitamin matched that of the cholecalciferol phosphate disodium synthesis up until the production of the yellow oil. The yellow THF oil (15 mL) and 6 mL of sodium hydroxide (3M) were added into the round bottom flask. Once a precipitate was formed, the THF layer was discarded and the precipitate transferred into a separation funnel using 20 mL hexane. The product was then extracted using one of three different methods.
i) The acid method: HCl 2M (total 15 mL) was added in two steps, adding 10 mL first and shaking, followed by additional 5 mL. The hexane layer was then collected and replaced with a further 20 mL hexane and the hexane extraction was repeated. This extraction was repeated for a third time (a total of 60 mL hexane added).
ii) The base-acid method: A series of aqueous solutions and hexane were added to the precipitate mixing after each addition: 50 mL of 3M NaOH, 30 mL hexane, 50 mL purified water, and 50 mL hexane. The pH was then adjusted to 2 by adding drops of concentrated HCl (12M), the layers were mixed vigorously and the layers were allowed to separate. The top hexane layer was collected. Two further washes with 100 mL hexane for the aqueous layer were performed, each time the top hexane layer was collected.
iii) The water-acid method: A further 80 mL hexane was added to the precipitate resulting in a total of 100 mL. Then 100 mL purified water was added. Then 5 mL of concentrated HCL (12M) was added, vigorous mixed, and the top layer was collected. Another 100 mL hexane was added along with 5 mL 10M NaOH and 0.5 mL strong HCl. Following a through mixing and layer separation, the collection of the top layer was then collected. A final 100 mL hexane was added and the top layer was collected.

The liquid-liquid extraction procedures were all followed by drying over MgSO₄ and rotary evaporating the hexane to produce a viscous resin like product which was considered the crude VDP acid form.

### 3.2.3 Characterization

### 3.2.3.1 Nuclear Magnetic Resonance (NMR) Spectroscopy

¹H, ¹³C and ³¹P NMR spectra were recorded using a DRX 400 (Bruker, UK) with Topspin software for data analysis. The cholecalciferol phosphate disodium or the acid form (6 mg) was dissolved in methanol-d₄ (600 μL), stirring overnight, and the starting material cholecalciferol (10 mg) was dissolved in CDCl₃ (600 μL). Proton NMR parameters include a sweep width of 8250.825 Hz, an acquisition time of 4.0 s, an interpulse delay time of 1 sec and 16 scans within a zg30 pulse program. Carbon NMR used a sweep width of 24038.461 Hz, an acquisition time of 1.4 sec, an interpulse delay time of 2 sec and 1000 scans all within a zgpg30 pulse program. Phosphorus NMR used a sweep width of 64724.918 Hz, an acquisition time of 0.5 sec, an interpulse delay time of 2 sec and 16 scans all within a zgpg30 pulse program.

### 3.2.3.2 Liquid Chromatography Mass Spectrometry Analysis

LC/MS analysis was performed using two different machines.

The cholecalciferolphosphate disodium analysis was carried out using a 1100 series HPLC with a G1322A Degasser (Agilent, USA) attached to a mass spectrometer (LCQ DECA XP, USA) using a Phenomenex Luna C18 (50×2 mm, 2.5 μm particle size, 100 Å diameter). The ion trap was operated in positive electrospray ionisation (ESI) mode. The sample concentration was 1 mg/mL, prepared in 45% acetonitrile (ACN), 45% water, 0.1% formic acid, and 10.7% methanol. The mobile phase was carried out as a gradient between 0.1% TFA in water and 0.1% TFA in ACN, starting with a 50:50 volume ratio and ending with a 98:2 by the end of a run time of 14.5 min. The column temperature was 45° C., a flow rate of 0.2 mL/min, and ion source temperature of 350° C.

The cholecalciferolphosphate acid form was analysed on a Waters Xevo TQ LC-MS/MS. The liquid chromatography utilised a biphenyl microbore 50 mm length×2.1 mm i.d. 5 μm Phenomenex Kinetix column. The ion trap was operated in positive electrospray ionisation (ESI) mode. The sample was prepared at 1 mg/mL in 90:10 volume ratio of methanol:formic acid 0.1%. A gradient method was employed using mobile phase A containing 70% acetonitrile, 10% water, 0.1% formic acid and 20% methanol and mobile phase B containing 80% water, 0.1% formic acid and 20% methanol. The sample injection volume was 10 μL and the runtime was 18 min. The autosampler was at 8 □C, column oven temperature of 40° C., a flow rate of 0.3 mL/min, and ion source temperature of 350° C.

### 3.2.3.3 HPLC Analysis

A HPLC method was established for the vitamin D, its phosphorylated products in order to confirm the product purity and characterise the chemical stability in transdermal formulations.

HPLC analysis was carried out using an HPLC system consisting of an auto-sample injector (Jasco AS-4050, Japan), a pump (Jasco PU-4180, Japan), and a photodiode array detector (Jasco MD-4017, Japan). The data was collected and analysed using the ChromNAV software. The HPLC column was a Phenomenex C18 Synergi Hydro-RP-column (250×4.6 mm, 4 μm particle size, 80 Å diameter) with a Phenomenex-AQ C18 guard a for cholecalciferol and a Phenomenex Kinetex Biphenyl column (250×4.6 mm, 5 Å diameter) for cholecalciferol phosphate. The mobile phase was ACN:MeOH (55:45) for cholecalciferol and MeOH:PBS pH 3.2 (90:10) for cholecalciferol phosphate (disodium and acid). Both mobile phases were filtered through a nylon membrane filter and degassed using a sonicator.

The injection volume was 20 μL, the flow rate for cholecalciferol was 2.0 mL/min and it was 1.0 mL/min for cholecalciferol phosphate (disodium and acid). A UV detection wavelength of 265 nm was employed with run times of 10 min for cholecalciferol and cholecalciferol phosphate disodium salt and 15 min for cholecalciferol phosphate acid. Standards in the range of 1-100 g/mL, 3.1-2000 g/mL and 3.1-1000 g/mL were produced using stock solutions of cholecalciferol in MeOH, cholecalciferol phosphate disodium in MeOH water (90:10) and cholecalciferol phosphate acid in MeOH water (90:10) respectively.

The HPLC method was verified through confirmation of the peak symmetry, assay calibration linearity, assay precision, limit of detection and limit of quantification. There was no analytical standard for the products to calculate purity, hence this was determined as a percentage of total peak area in the chromatogram using a diode array detector.

### 3.3 Part 2: Pre-Formulation Studies

### 3.3.1 Solubility Studies

Aliquots of cholecalciferol, cholecalciferol phosphate disodium, and cholecalciferol phosphate acid were added to a range of solvents including propylene glycol (PG), distilled water, methanol, ethyl acetate, ethanol, n-hexane, and the visual solubility was determined. A precise assessment of the saturation solubility was for some of the solvent systems was made using high-performance liquid chromatography (HPLC). In the HPLC solubility studies, an excess of each solute was added into 2 mL of each test solvent. The solutions were left stirring overnight at room temperature, (RT), 22° C. The saturation of the solutions was confirmed by the visual appearance of crystals and the drug suspensions were filtered through 0.2 μm cellulose acetate syringe filters, diluted with excess of mobile phase, and the solute concentration of each sample was assessed using HPLC.

### 3.3.2 Cholecalciferol vs Cholecalciferol Phosphate Disodium Skin Permeation

In order to assess the effect of adding phosphate to vitamin D chemical structure on the penetration of vitamin D through the skin porcine skin permeation studies were performed. Using the data from the pre-formulation studies solutions of cholecalciferol and cholecalciferol phosphate disodium, were prepared. The permeation of the cholecalciferol and cholecalciferol phosphate from the PG saturated solutions were compared using previously validated Franz cell method based on the principles of the FDA, SUPAC-SS guidelines [FDA (CDER), 1997, Guidance for industry—SUPAC-SS Non-sterile Semisolid Dosage Form, Scale-up and post approval changes: chemistry, manufacturing and controls; in vitro release testing and in vivo bioequivalence documentation] (FIG. 4). The developed HPLC methods were used for quantification.

The Franz cell methodology was also used to study how the vitamin D is deposited into the skin with extraction from the three layers of the skin being verified using a recovery measurement using spiked samples.

### 3.3.2.1 Franz Cell Method Set Up and Verification Studies

To assess the chemical stability of the test agents in the liquids in which they were to be dissolved for the skin permeation experiments, 1 mg/mL solutions of cholecalciferol and cholecalciferol phosphate disodium in propylene glycol (PG) were prepared and incubated at 37° C. for 24 h. In addition, 100 μg/mL cholecalciferol in 50:50 PG:ethanol mixture, 100 μg/mL cholecalciferol phosphate disodium in 90:10 methanol:water mixture as well as and in 50% PG 40% Methanol: 10% water pH 3.2 (90:10) were also incubated at 37° C. for 24 hours.

To verify the percentage of drug that could be recovered from each layer of porcine skin, a 2.19±0.21 cm² piece of the skin was cut from a prepared piece of porcine ear. Tape stripping was used to isolate the Stratum corneum (SC). This was achieved by placing a 300 g weight to press a piece of adhesive tape (Scotch 845 book tape, 3M, Bracknell, UK) to the surface of the skin for 10 sec. The tape was removed, and this removed one layer of the SC. This process was repeated until 20 layers of skin were taken. These strips were added into sample bottles. The epidermis was removed from the dermis physically using a scalpel and both the separated layers were cut into small pieces, and placed into bottles.

The test solutions (1 mg/mL of cholecalciferol or cholecalciferol phosphate disodium in PG) were spiked (500 μL) into a sample bottle containing the SC and (200 μL) into the sample bottles containing the epidermis and dermis. The samples containing the spiked test agents were left for 1 h for the agents to absorb into the tissues. Afterwards, 10 mL of extraction fluid (50:50 PG:ethanol for cholecalciferol and 90:10 MeOH:water for cholecalciferol phosphate disodium) was added into SC, and 2 mL of extraction fluid was added into epidermis and dermis then incubated in a shaking water bath overnight at 32° C. The extraction media with both epidermis and dermis were then homogenised by Ultra Turrax for 1 min, filtered using 0.2 m syringe filters, and analysed by HPLC method. The SC samples were filtered and analysed also using the HPLC method.

3.3.2.2 Permeation Studies from Propylene Glycol Solutions

To study if the addition of the phosphate to cholecalciferol facilitated enhanced transdermal permeation, donor solutions were prepared by adding 2.5 mg/mL of cholecalciferol or cholecalciferol phosphate disodium in 100% PG. Note the acid form of cholecalciferol phosphate disodium was not tested in this experiment. On the day of the experiment the skin was defrosted, and the subcutaneous fat was manually removed using a scalpel. The tissue was cut into the appropriate size sections (0.25 mm thick, 2.19±0.21 cm²) and mounted with the SC facing the donor compartment between donor and receiver compartments of Franz diffusion cells. Each pieces of skin was sealed between the two compartments using parafilm (Bemis Company, Neenah, WI, and 12 mm magnetic stir bars were placed into the receiver compartment. The receiver compartment was filled with 50:50 PG:mobile phase (the mobile phase was made of 55:45 acetonitrile:ethanol for cholecalciferol and 90:10 methanol:water pH 3.2 for cholecalciferol phosphate disodium). The cell sealing was confirmed by cell inversion and monitoring solvent back diffusion. Sink conditions were maintained throughout the transport assays as the drug concentration did not exceed 10% of its statured solubility. The temperature of the Franz diffusion (2.2 cm²) was maintained at 37° C. in a water bath, to achieve a skin temperature 32° C. After 1 h equilibration at 37° C. in the water bath, 1 mL of receiver fluid was taken (time point=0 min) and a fresh receiver solution was replaced to keep the volume constant, and 1 mL of each formulation was applied in the donor compartment. After 24 h, the receiver fluid was collected and analysed by HPLC methods.

3.3.2.3 Deposition from Propylene Glycol Solutions

At the end of the permeations studies, the Franz cells were dismantled, and the donor solutions were collected into bottles containing 10 mL of extraction fluid; PG:EtOH (50:50) for cholecalciferol and MeOH:water (90:10) for cholecalciferol phosphate disodium. This was completed by wiping the surface of the skin and inside of the donor compartment with two wet and then two dry cotton buds. The SC was removed from the epidermis by tape stripping (as mentioned above). The first two pieces of tape were added into the donor solution, as the first 2 strips were considered as part of the applied formulation and its removal was part of the formulation wash off. Then, the next 20 pieces of tapes were added into bottles (corresponding to the SC) containing 10 mL of extraction fluid. The epidermis was removed from the dermis and cut into small pieces by scalpels, placed in bottles containing 2 mL of extraction fluid. The dermis layer was cut into small pieces was placed in bottles containing 2 mL of extraction fluid. All skin samples were incubated with extraction fluids (as detailed above) for the surface wash and the samples were placed in a shaking water bath overnight at 32° C., which is the normal skin surface temperature.

Following homogenising solution with epidermis and dermis for 1 min by Ultra Turrax, solutions were filtered through 0.2 μm syringe filters of each bottle was collected and analysed by the HPLC method. The amount of cholecalciferol and cholecalciferol phosphate disodium on the surface of the skin, deposited in the SC, epidermis, dermis, and passed into the receiver fluid was determined. The percentage of mass recovery per cm² of skin was calculated, and the drug deposition profile could be obtained.

### 3.4 Part 3. Patch Development

### 3.4.1 Cholecalciferol Phosphate (Disodium and Acid) Patch Development

Pre-formulation studies were performed to determine the compatibility of cholecalciferol phosphate (disodium and acid forms) with a range of adhesives (Table 38) that could be used to manufacture of the patch. The compatibility of the drugs with the patch excipients was assessed through checking the clarity of the mixture. The adhesives that did not dissolve the drug with or without addition of co-solvents were eliminated from the study and initial formulations were designed from those remaining adhesives that facilitated acceptable solubility.

TABLE 38
Type of patch adhesives and their properties.

| Type of | Name of | | Solvent |
|---|---|---|---|
| adhesive | adhesive | Properties | Compatibility |
| Polyisobutene | Oppanol | Neutral | Hexane |
| | B12 | Low | |
| | (BASF) | molecular | |
| | | weight | |
| | Oppanol | Neutral | |
| | N100 | High | |
| | (BASF) | molecular | |
| | | weigh | |
| Acrylates | DURO- | No functional | Ethyl |
| copolymer | TAK | groups | acetate/methanol |
| (pressure | 87-9301 | 36.5% solids | PG |
| adhesives) | (Henkel) | | Isopropanol |
| | | | Propanol |
| | DURO- | —OH | Ethyl acetate |
| | TAK | functional | Ethyl acetate/PG |
| | 87-2516 | group | Ethyl acetate/ |
| | (Henkel) | Contains cross | methanol |
| | | linker | |
| | | 41.5% solids | |
| | DURO- | —COOH | Hexane |
| | TAK0 | functional | |
| | 87-2052 | group | |
| | (Henkel) | Contains cross | |
| | | linker | |
| | | Contains vinyl | |
| | | acetate | |
| | | 47.5% solids | |
| Amine- | BIO-PSA | Low silicon | Hexane |
| Compatible | 7-4302 | content, 60% | |
| Silicone | (Dupont) | solid content | |
| | | in ethyl acetate | |

The compatibility of the adhesive with solvents and drug was established. The maximum drug loading in the adhesive following solvent evaporation was determined by mixing the drug, adhesive and co-solvents until a clear solution is obtained at different drug:adhesive ratios by placing a drop of solution onto patch backing sheet, leaving the slide to dry for 3-4 hours and examining the formed fil on the surface of the backings for evidence of drug crystals were assessed using KKmoon digital magnifier with LED light (China). Following patch optimization, the drug-adhesive solution was cast as 10×10 cm squares using of backing (3M™ Scotchpak™ Polyester Backing Film Laminate 9730). A sufficient volume of the adhesive was applied to achieve a target coating of 8 mg/cm². Following complete drying of the solvents, the adhesive layer was covered by M3 Release liner (SCOTCHPAK 9755), compressed, and then cut using 34 mm circular manual die cutter (SCP Super Cutting Press).

### 3.4.2 Patch Drug Release Testing Method Development

Preliminary studies were conducted to determine the best synthetic membrane for the patch release using the Franz diffusion cells. Regenerated cellulose (12-14 k molecular weight cut-off Medicell International UK) was compared to nylon (0.20 μm, Whatman, UK) using a range of donor and receiver solutions. The regenerated cellulose membrane was heated to 60° C. for 30 minutes after which the double layer of polymer (it is supplied as a dialysis membrane) was separated and mounted onto the Franz cell (FIG. 3) by sealing between the two compartments using parafilm, and 12 mm magnetic stirrers were placed into the receiver compartment. The receiver compartment was filled with either 50:50 PG:mobile phase (90:10 methanol:water pH 3.2), or 80:20 PG:distilled water, or 75 mM pH 7 saline. The cell sealing was confirmed by cell inversion and monitoring solvent back diffusion. Sink conditions were maintained throughout the transport assays because the active concentration did not exceed 10% of its saturated solubility in the receiver fluid throughout the experiments. The temperature of the Franz diffusion was maintained at 37° C. in a water bath, to set up the membrane temperature to 32° C. After 1 h equilibration at 37° C. in the water bath, 1 mL of receiver fluid was taken (time points 0 min) and a fresh receiver solution was replaced to keep the volume content, and 1 mL of cholecalciferol phosphate disodium saturated formulation was applied in the donor compartment (PG or in saline). The 1 ml receiver fluid samples were collected and analysed by HPLC methods at different time intervals. Note: both the nylon and regenerated cellulose membranes were suitable for release testing, initially nylon was used for the testing, but this was switched to regenerated cellulose when it was discovered the release from the patches was low because regenerated cellulose allowed the active to pass the membrane slightly faster.

### 3.4.3 Cholecalciferol Phosphate Disodium Patch Release Testing

To assess if cholecalciferol phosphate disodium could be release from an ‘active-in-adhesive’ patch a single patch was produced using DUROTAK 387-2516 adhesive because it was found to be the only compatible adhesive in the pre-formulation studies. The patch was produced by mixing 82.9 μL of the adhesive, 1.92 μL of PG and the 4 mg of cholecalciferol phosphate disodium (Table 39). The patch formulation was cast into an in-house rectangular tray measuring 10 by 10 cm. The mixture was left to cure for 3-4 h. The laminate was then added to the adhesive and circles of 34 mm diameter were cut using a dye cutting machine and the release of the cholecalciferol was assessed using the Franz diffusion cells fitted with a nylon membrane over 24 h. A nylon membrane (0.20 μm) was glued to the receiver chamber and no donor chamber was employed to allow the patch to be placed directly on the surface of the nylon membrane. A cholecalciferol phosphate disodium saturated PG solution was used as a comparator with the standard Franz cell set-up previously described. The receiver fluid was 80% PG 20% water for both release testing from the solution and the patch.

TABLE 39
Cholecalciferol phosphate disodium patch compositions.

| | | | | Dry | Theoretical |
|---|---|---|---|---|---|
| | | | | weight | Patch |
| | | Ingredient | | (per | Composition |
| Patch | Ingredient | conversion | | 100 mg) | (%) |
| F1 | DURO-TAK | 207 | μL | 68 | 85 |
| | 387-2516 | | | | |
| | PG | 4.8 | μL | 5 | 5 |
| | VDP Disodium | 10 | mg | 10 | 10 |
| | Solvent: Methanol/ | 3.5/6.9/0.375 | | | |
| | Ethyl acetate/Water | mL | | | |
| | Total | 10.77 | mL | 100 | 100% |

### 3.4.4 Cholecalciferol Phosphate Acid Release Testing

To assess if cholecalciferol phosphate disodium could be release from an ‘active-in-adhesive’ patch a total of six different patch formulations were made using the adhesive, Polyisobutene, Oppanol B12, Duro-Tak 9301 and Duro-Tak 2516 (Table 40). Duro-tak dissolves almost instantly however Oppanol B12 takes about one hour. The patch formulation was cast into an in-house rectangular tray measuring 10 by 10 cm. The mixture was left to cure for 3-4 h. The laminate was then added to the adhesive and circles of 34 mm diameter were cut using a dye cutting machine and the release of the cholecalciferol was assessed using the Franz diffusion cells fitted with a cellulose membrane over 24 h as described above for the sodium form of the active. A regenerated cellulose membrane was employed in the Franz cells for the cholecalciferol phosphate acid release testing as the drug saturated solution experiments had demonstrated that it allowed more cholecalciferol to pass through it compared to nylon thus making the release experiment more sensitive. The regenerated cellulose membrane was heated to 60° C. for 30 minutes after which the double layer was separated and mounted onto the Franz cell (FIG. 4) by sealing between the two compartments using parafilm, and 12 mm magnetic stirrers were placed into the receiver compartment that contained the receiver fluid, 50:50 PG:mobile phase (90:10 Methanol:water pH 3.2), which was modified from the disodium release studies to facilitate more cholecalciferol to pass the membrane and thus make the testing more sensitive. The temperature of the Franz diffusion was maintained at 37° C. in a water bath, to set up the membrane temperature to 32° C. Receiver fluid was collected after 24 hours and analysed by the developed HPLC method.

TABLE 40
Cholecalciferol phosphate acid patch compositions for the cellulose
acetate release studies.

| | | | | | | Theoretical |
|---|---|---|---|---|---|---|
| | | Weight | | | | Patch |
| | | (per tray of | | Ingredient | | Composition |
| Patch | Ingredient | 100 cm²) | | conversion | | (% w/w) |
| Oppa | Oppanol B12 | 1,200 | mg | 1,200 | mg | 95% |
| (F2) | VDP acid | 65 | mg | 65 | mg | 5% |
| | Hexane | 16.4 | g | 24.5 | mL | |
| Oppa-Double | Oppanol B12 | 1,200 | mg | 1,200 | mg | 95% |
| Thickness | VDP acid | 65 | mg | 65 | mg | 5% |
| (F3) | Hexane | 16.4 | g | 24.5 | mL | |
| Oppa | Oppanol B12 | 1,135 | mg | 1,135 | mg | 95% |
| Enhancer | VDP acid | 65 | mg | 65 | mg | 5% |
| (F4) | Dodecylamine | 65 | mg | 65 | mg | |
| | Hexane | 16.4 | g | 24.5 | mL | |
| Duro-Tak | VDP acid | 67.1 | mg | 5 | mg | 8.4% |
| 87-9301 | Dodecylamine | 67.1 | mg | 5 | mg | 8.4% |
| (F5) | Duro-Tak | 655 | mg | 1.82 | mL | 83.2% |
| | 9301 | | | | | |
| | Methanol | | | 6.7 | mL | |
| | Ethyl Acetate | | | 3.35 | mL | |
| Duro-Tak | VDP acid | 81 | mg | 10 | mg | 10% |
| 87-2052 | Duro-Tak | 718.7 | mg | 2.18 | mL | 90% |
| (F6) | 9301 | | | | | |
| | Ethyl Acetate | | | 0.4 | mL | |
| | Hexane | | | 0.5 | mL | |
| Duro-Tak | VDP acid | 36.36 | mg | 2 | mg | 4.55% |
| 87-2516 | Dodecylamine | 36.36 | mg | 2 | mg | 4.55% |
| (F7) | Duro-Tak | 727 | mg | 1.75 | mL | 90.9% |

| | | |
|---|---|---|
| 9301 | | |
| Methanol | 3.6 | mL |
| Ethyl Acetate | 18.2 | mL |

### 3.4.5. Cholecalciferol Phosphate Acid Patch Skin Permeation and Deposition

3.4.5.1 Cholecalciferol Phosphate Acid Franz cell Method Set Up and Verification Studies

To assess the chemical stability of the cholecalciferol phosphate acid in the solutions employed in the skin permeation experiments a 1 mg/mL solution of cholecalciferol phosphate acid in propylene glycol (PG) was prepared and incubated at 37° C. for 24 hours. In addition 100 μg/mL cholecalciferol phosphate acid in 90:10 methanol:water and in 80:20 PG:water were also incubated at 37° C. for 24 hours. The concentration pre and post incubation was compared to determine the chemical stability during the permeation studies.

To verify the percentage of drug that could be recovered from the skin dermatomed porcine skin (500 μm thickness) was used. The test solution (1 mg/mL of cholecalciferol phosphate acid in PG) was spiked (200 μL) onto the surface of the 9 cm² of dermatomed skin (n=3). The skin was left for 1 h for the drug to absorb into the tissues. After the 1 h incubation period, 2 mL of extraction fluid (MeOH:water, 90:10) was added. The samples were transferred to Eppendorf tubes with the addition of one metal ball and loaded into the TIssuLyser II (Qiagen) for 15 minutes at 30 Hz. The samples were then centrifuged (5 minutes at 13 krpm) and supernatant assayed.

### 3.4.5.2 Cholecalciferol Phosphate Acid Patch Manufacture

Two different patch formulations were made using the adhesive, DURO-TAK 2052 (Table 41). Formulation 1 was produced by mixing 1.3 mL of the adhesive, 3 mL hexane, 2 mL ethyl acetate and 60 mg of cholecalciferol phosphate acid, while formulation 2 had an addition 36 μL of Transcutol (Table 41). The patch formulations were cast into an in-house rectangular tray measuring 10×10 cm. The mixture was left to cure for 3-4 h. The laminate was then added to the adhesive and circles of 34 mm diameter were cut using a dye cutting machine and the release of the cholecalciferol was assessed using the Franz diffusion cells.

TABLE 41
Cholecalciferol phosphate acid patch compositions for the cellulose
acetate release studies.

| | | Weight | | Weight | | | | Theoretical |
|---|---|---|---|---|---|---|---|---|
| | | (per tray | | (per | | | | Patch |
| | | of 100 | | 100 | | Ingredient | | Composition |
| Patch | Ingredient | cm²) | | mg) | | conversion | | (%) |
| DT 87- | VDP acid | 70.85 | | 60 | mg | 60 | mg | 8.9% |
| 2052 | DT 9301 | 1.54 | mL | 617 | mg | 1.3 | mL | 91.1% |
| (F8) | Ethyl | 2.36 | mL | | | 2 | mL | |
| | Acetate | | | | | | | |
| | Hexane | 3.54 | mL | | | 3 | mL | |
| DT 87- | VDP acid | 67.3 | mg | 60 | mg | 60 | mg | 8.5% |
| 2052 | DT 2052 | 1.46 | mL | 617 | mg | 1.3 | mL | 86.5% |
| Transcutol | Transcutol | 40 | μL | 35 | μL | 35 | mg | 5% |

| | | | | | |
|---|---|---|---|---|---|
| (F9) | Ethyl | 2.25 | mL | 2 | mL |
| | Acetate | | | | |
| | Hexane | 3.37 | mL | 3 | mL |

### 3.4.5.3 Cholecalciferol Phosphate Acid Patch Skin Permeation Studies

To perform the skin permeation studies dermatomed skin (500 μm) was mounted into Franz cells by sealing between the two compartments using parafilm, and 12 mm magnetic stirrers were placed into the receiver compartment that contained the receiver fluid, 80:20 PG:water. The temperature of the Franz diffusion was maintained at 37° C. in a water bath, to set up the skin temperature to 32° C. Receiver fluid was collected at 2, 5, 21, 22 and 24 hours and analysed by HPLC method. The skin deposition was determined by removing the dermatomed skin from the donor compartment when the 24 h was completed and incubating it with 2 mL of extraction fluid (MeOH:water, 90:10). The samples were then transferred to Eppendorf tubes with the addition of one metal ball and loaded into the TissuLyser II (Qiagen) for 15 minutes at 30 Hz. The samples were then centrifuged (5 minutes at 13 k rpm) and supernatant assayed.

### 3.5 Results

### 3.5.1 Part 1. Phosphorylated Vitamin D Synthesis and Characterisation

### 3.5.1.1 Cholecalciferol Phosphate Disodium Synthesis and Characterization

The synthesis of cholecalciferol phosphate disodium was repeated three times to obtain 3 batches of cholecalciferol phosphate. The obtained products were analysed using LC-MS and NMR, these techniques were used to provide information about the molecular weight, structure, identity and quantity of sample components.

### Nuclear Magnetic Resonance (NMR) Spectroscopy

NMR analysis was conducted to confirm the structure of the synthesised cholecalciferol phosphate disodium. The phosphorous NMR spectra for all three batches showed one doublet at approximately 5 ppm which indicated that the monomeric form of the cholecalciferol phosphate disodium was formed, and no bis-cholecalciferol phosphate was present. In the carbon NMR, 26 peaks were identified for the starting material cholecalciferol (Table 42). All three batches of the cholecalciferol phosphate disodium generated the 26 peaks identified in the starting material. Since all the 26 peaks of the starting material were identified in the final products confirmed that the synthetic procedure did not modify the vitamin structure. In the proton NMR, the most important protons of cholecalciferol to track in the reaction, the protons attached to C1 and C2. They were an octat peak ¹C—H (at 3.95 ppm), a doublet of doublets ²C—H₁ (at 2.82 ppm) and a doublet of doublets ²C—H₂ (at 2.58 ppm) in the starting material (data not shown). The cholecalciferol phosphate disodium proton NMR displayed a multiplet peak at 4.2 ppm, which was assigned to be the proton of C1. This peak had changed compared to the starting material as expected, due to the introduction of the phosphorous group. The 2 protons of C2 were shifted to 2.8 ppm and 2.7 ppm from 2.82 ppm and 2.58 ppm, as a result of the addition of phosphorous group. These results demonstrated that phosphate had been introduced into cholecalciferol phosphate at the targeted alcohol functionality.

TABLE 42
Assignment of carbon NMR spectrum of the starting material
cholecalciferol and cholecalciferol phosphate disodium.

| | Chemical shift (ppm) | | | |
|---|---|---|---|---|
| | | Cholecalciferol phosphate | | |
| Atom | | disodium | | |
| number | Cholecalciferol | Batch 1 | Batch 2 | Batch 3 |
| C1 | 69.21 | 72.92 | 72.92 | 72.93 |
| C2 | 40.55 | 41.98 | 41.98 | 41.98 |
| C3 | 135.02 | 138.57 | 138.56 | 138.57 |
| C4 | 145.10 | 147.95 | 147.94 | 147.95 |
| C5 | 28.03 | 29.15 | 29.15 | 29.15 |
| C6 | 31.94 | 34.10 | 34.11 | 34.10 |
| C7 | 112.43 | 111.75 | 111.77 | 111.78 |
| C8 | 122.49 | 122.37 | 122.38 | 122.38 |
| C9 | 117.47 | 119.27 | 119.26 | 119.25 |
| C10 | 142.37 | 141.73 | 141.75 | 141.74 |
| C11 | 29.04 | 29.96 | 29.96 | 29.97 |
| C12 | 22.57 | 23.17 | 23.17 | 23.17 |
| C13 | 35.17 | 36.16 | 36.18 | 36.16 |
| C14 | 45.94 | 46.89 | 46.89 | 46.89 |
| C15 | 12.01 | 12.34 | 12.34 | 12.34 |
| C16 | 56.36 | 57.54 | 57.54 | 57.54 |
| C17 | 22.83 | 23.25 | 23.25 | 23.25 |
| C18 | 23.88 | 24.97 | 24.97 | 24.97 |
| C19 | 56.61 | 57.99 | 57.99 | 57.99 |
| C20 | 18.86 | 19.38 | 19.38 | 19.38 |
| C21 | 36.15 | 37.45 | 37.45 | 37.45 |
| C22 | 39.52 | 40.68 | 40.68 | 40.68 |
| C23 | 23.60 | 24.58 | 24.59 | 24.59 |

### Liquid Chromatography Mass Spectroscopy Analysis

LC/MS analysis was conducted to confirm the mass thus the identity of the synthesised cholecalciferol phosphate. The mass spectrum was generated in positive ESI mode, thus the molecular ions (m/z) are expressed as [M+H]⁺. The starting material showed only one peak in the liquid chromatogram at a retention time of 13.98 min, which had a m/z of 385.24 g/mol. As the calculated m/z of the starting material was 384.64 g/mol, the peak on the chromatogram was assigned as the starting material cholecalciferol.

Only 2 of the 3 batches of the cholecalciferol phosphate were analysed, both produced only one peak at a retention time of 11.65 min (batch 1), and 11.52 min (batch 2). These peaks did not match the retention time of the starting material and demonstrated the synthesised product was more hydrophilic than the starting material. The chromatogram peaks gave a m/z 466.25 (batch 1), and m/z 465.15 (batch 2). As the calculated m/z of the cholecalciferol phosphate is 464.63 g/mol, it demonstrated that all final products were cholecalciferol phosphate. With respect to purity, batch 1 showed a shoulder peak, resulting in % purity of 87%, while batch 2 showed a higher % purity of 97% with a much smaller shoulder peak. With respect to % yield it was 99 and 66% respectively.

### HPLC Analysis

The UV absorbance maximum for cholecalciferol phosphate in the optimised mobile phase was 265 nm, which was used as the detection wavelength throughout the study. The calibration curves were linear over the concentration range of 1-100 μg/mL and 3.1-2000 μg/mL for cholecalciferol and cholecalciferol phosphate disodium, respectively. Limits of detection (LoD) and quantification (LoQ) for cholecalciferol were calculated at 0.59 μg/mL and 1.98 μg/mL, respectively. LoD and LoQ were calculated at 13.81 μg/mL and 46.06 μg/mL for cholecalciferol phosphate disodium, respectively (Table 43).

TABLE 43
Method validation parameters of the HPLC for cholecalciferol and
cholecalciferol phosphate disodium.

| | | | |
|---|---|---|---|
| | | | Cholecalciferol |
| | ICH | | phosphate |
| Validation parameter | level/limit | Cholecalciferol | disodium |

System suitability

| | | | |
|---|---|---|---|
| Linearity range (μg/mL) | — | 1.5-100 | 3.1-2000 |
| Linearity (R², n = 3) | >0.999 | □ | □ |
| Peak symmetry | <2, | □ | □ |
| As (n = 3 ± SD) | Ideal | | |
| | As = 1 | | |
| Theoretical plate number | >2000 | □ | □ |
| N (n = 3 ± SD) | | | |
| LoD (μg/mL) | — | 0.59 | 13.81 |
| LoQ (μg/mL) | — | 1.98 | 46.06 |
| Accuracy % (n = 3 ± SD) | 95-105% | □ | □ |
| | | (100.3□2.5) | (98.4□2.1) |

Precision

| | | | |
|---|---|---|---|
| Intra-day variability | <2% | □ | Not calculated |
| (repeatability, % CV) | | (1.2 ± 0.5) | |
| Inter-day variability | <2% | □ | Not calculated |
| (intermediate precision, | | (0.7 ± 0.3) | |
| % CV) | | | |

### Cholecalciferol Phosphate Acid Synthesis and Characterization

The synthesis was repeated three times to obtain 3 batches of cholecalciferol phosphate acid form. The obtained product was analysed using LC-MS and NMR, these techniques were used to provide information about the molecular weight, structure, identity and quantity of sample components.

### Nuclear Magnetic Resonance (NMR) Spectroscopy

NMR analysis was conducted to confirm the structure of the synthesised cholecalciferol phosphate acid. The phosphorous NMR spectra for all three batches showed one doublet at approximately 0 ppm which indicated phosphate was present. In the carbon NMR, 26 peaks were identified for the starting material cholecalciferol. The cholecalciferol phosphate acid had those 26 peaks (Table 44), in addition to some more peaks that were assigned to the solvents used during synthesis or some impurities. Since all 26 peaks of the starting material were identified in the final product, the synthetic procedure did not degrade the vitamin's structure. In the proton NMR, the protons attached to C1 and C2 were also identified in cholecalciferol phosphate acid. It displayed a multiplet peak at 3.3 ppm which was assigned to be the proton of C1. The 2 protons of C2 were shifted to 2.0-2.5 ppm.

TABLE 44
Assignment of carbon NMR spectrum of the starting material
cholecalciferol and Cholecalciferol phosphate acid.

| | Chemical shift (ppm) | | | |
|---|---|---|---|---|
| Atom | Cholecal- | Cholecalciferol phosphate acid | | |
| number | ciferol | Batch 1 | Batch 2 | Batch 3 |
| C1 | 69.21 | 72.95 | 56.59 | 74.03 |
| C2 | 40.55 | 44.21 | 40.51 | 40.50 |
| C3 | 135.02 | 135.74 | 135.27 | 135.05 |
| C4 | 145.10 | 145.76 | 145.45 | 145.31 |
| C5 | 28.03 | 33.99 | 27.76 | 31.70 |
| C6 | 31.94 | 35.92 | 31.80 | 33.70 |
| C7 | 112.43 | 110.98 | 111.24 | 111.36 |
| C8 | 122.49 | 121.54 | 121.77 | 123.14 |
| C9 | 117.47 | 117.63 | 117.54 | 117.51 |
| C10 | 142.37 | 140.97 | 141.26 | 141.38 |
| C11 | 29.04 | 34.03 | 28.59 | 33.65 |
| C12 | 22.57 | 27.36 | 21.78 | 23.57 |
| C13 | 35.17 | 36.06 | 35.90 | 35.92 |
| C14 | 45.94 | 45.52 | 46.96 | 46.96 |
| C15 | 12.01 | 22.30 | 10.94 | 21.88 |
| C16 | 56.36 | 56.13 | 48.24 | 56.13 |
| C17 | 22.83 | 27.76 | 21.88 | 27.35 |
| C18 | 23.88 | 31.35 | 23.57 | 28.58 |
| C19 | 56.61 | 56.58 | 56.13 | 56.59 |
| C20 | 18.86 | 23.20 | 17.97 | 22.30 |
| C21 | 36.15 | 39.28 | 36.06 | 36.06 |
| C22 | 39.52 | 40.53 | 39.28 | 39.28 |
| C23 | 23.60 | 28.57 | 23.21 | 27.76 |
| C24 | 45.87 | 44.25 | 45.54 | 45.54 |
| C25 | 27.67 | 32.02 | 27.36 | 31.35 |
| C26, 27 | 22.27 | 23.57 | 21.54 | 23.22 |
| Solvents | | 46.96, 47.17, | 48.03, 47.88, | 34.51, 43.84, |
| (Triethylamine/ | | 47.38, 47.60, | 47.81, 47.60, | 43.88, 47.17, |
| Hexane) | | 47.67, 47.81, | 47.39, 47.18 | 47.39, 47.60, |
| | | 47.88, 48.02, | | 47.81, 47.88, |
| | | 48.09, 48.24, | | 48.02, 48.10, |
| | | 48.31 | | 48.24, |
| Impurity | | | | 74.09, 121.87 |

### Liquid Chromatography Mass Spectroscopy Analysis

Batches 1, 2 and 3 of cholecalciferol phosphate acid had a main peak in the liquid chromatogram at a retention time of 9.83 min, 8.93 min and 9.21 min respectively, which did not match the retention time of the starting material nor the salt form. The acid synthesised product was even more hydrophilic than the starting material and the salt counterpart. With respect to purity, batch 1 main peak was equivalent to 92% with two more peaks between 12 and 13.5 min accounting for 2 and 6% respectively. Batch 2 also had a main peak equivalent to 92% with one later peak at 12 min accounting for the remaining 8%. Batch 3 main peak was 80% with two later peaks between 12 and 12.6 min accounting for 5% and 15% respectively. The mass spectrum was performed by positive ESI mode, thus the molecular ions (m/z) are expressed as [M+H]⁺. The calculated m/z of the cholecalciferol phosphate is 464.63 g/mol and all three batches had an m/z of 465.35 corresponding to the parent.

### HPLC Analysis

The calibration curve was linear over the concentration range of 3.1-1000 μg/mL for cholecalciferol phosphate acid. Limit of detection (LoD) and (LoQ) were calculated at 8.05 μg/mL and 26.84 μg/mL, respectively (Table 45).

TABLE 45
Method validation parameters of the HPLC for
cholecalciferol phosphate acid.

| | | |
|---|---|---|
| | ICH | Cholecalciferol |
| Validation parameter | level/limit | phosphate acid |

System suitability

| | | |
|---|---|---|
| Linearity range (μg/mL) | — | 3.1-1000 |
| Linearity (R², n = 3) | >0.999 | □ |
| Peak symmetry | <2, | □ |
| As (n = 3 ± SD) | Ideal | |
| | As = 1 | |
| Theoretical plate number | >2000 | □ |
| N (n = 3 ± SD) | | |
| LOD (μg/mL) | — | 8.05 |
| LOQ (μg/mL) | — | 26.84 |
| Accuracy % (n = 3 ± SD) | 95-105% | |

Precision

| | | |
|---|---|---|
| Intra-day variability | <2% | □ |
| (repeatability, % CV) | | 1.02 ± 0.2 |
| Inter-day variability | <2% | □ |
| (intermediate precision, | | 0.41 ± 0.3 |
| % CV) | | |

### 3.5.2 Part 2: Pre-Formulation Studies

### 3.5.2.1 Solubility Studies

The solubility of cholecalciferol, cholecalciferol phosphate disodium and acid were tested in a range of solvents (Table 46).

TABLE 46
The solubility in a range of solvents (* visual solubility).

| Compound | Solvent | Solubility (mg/mL) |
|---|---|---|
| Cholecalciferol | PG | 7.8 ± 0.19 |
| | PG + 5% water | 2.4 ± 0.002 |
| | PG + 10% water | 1.1 ± 0.005 |
| | PG + 20% water | 0.7 ± 0.004 |
| Cholecalciferol | PG | 2.67 ± 0.31 |
| Phosphate disodium | PG + 5% ethanol | 0.34 ± 0.61 |
| Cholecalciferol | Ethyl acetate | <0.1* |
| Phosphate acid | Methanol(0.1% TFA) | <0.1* |
| | PG | ~10* |
| | Ethanol | ~4* |
| | Hexane | ~40* |

### 3.5.2.2 Cholecalciferol Vs Cholecalciferol Phosphate Disodium Skin Permeation and Deposition

### Franz Cell Method Set Up and Verification Studies

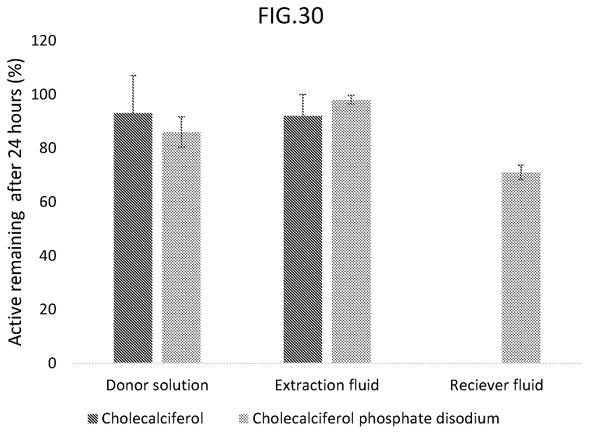

To establish a sound assay to assess the performance of the patches the chemical stability of cholecalciferol and cholecalciferol phosphate disodium in the solvents employed in the active release and skin permeation studies was assessed (FIG. 30). Three types of solvents were used, a donor solvent, an extraction solvent and a receiver solvent. The test actives were applied to the Franz cell systems in a PG donor solvent in some of the permeation and release studies because this was a solvent that could solubilise both actives. The solvent used to extract the test agents from the skin were 50:50 PG:ethanol for cholecalciferol and 90:10 methanol:water for cholecalciferol phosphate respectively because the actives showed good solubility in these solvents. The receiver solutions of 50:50 PG:mobile phase (90:10 methanol:water pH 3.2) for cholecalciferol phosphate disodium was selected as the active showed good solubility in this solution and it was thought more compatible with the skin compared to the extraction fluid. The chemical stability of cholecalciferol in the receiver fluid was not assessed as it was not expected that this agent would pass through the skin. The cholecalciferol appeared chemically stable in both fluids tested at 93±13% (PG) and 92±8% (extraction fluid), but the cholecalciferol phosphate appeared to be only stable in the extraction fluid (98±1.6%), but not in PG (86±5.7%) or the receiver fluid (71±2.7%). Considering the small magnitude of the loses compared to the biological variability of the experiments themselves no correction was applied to the data to account for them in the subsequent experiments.

The percentage of cholecalciferol and cholecalciferol phosphate disodium (in PG) recovered form skin was determined via and extraction study (Table 47). The measured drug extraction was within the required 100±15% recovery rate except for the cholecalciferol phosphate disodium epidermis recovery, which given the other data, which was considered to be an anomaly (Pharmaceutical Preparations and Organization 2005). No correction was applied to the data to account for the losses observed in the recovery studies as they were considered not to have a significant impact on the subsequent studies.

TABLE 47
Recovery of cholecalciferol and cholecalciferol phosphate
from porcine skin layers.

| Compound | Recovery in skin (%) | |
|---|---|---|
| Cholecalciferol | Stratum corneum | 114 ± 5 |
| | Epidermis | 92 ± 2 |
| | Dermis | 102 ± 4 |
| Cholecalciferol | Stratum corneum | 79 ± 7 |
| phosphate disodium | Epidermis | 14 ± 4 |
| | Dermis | 87 ± 14 |

Data represents the mean ± one standard deviation (n = 3).

### Permeation and Deposition

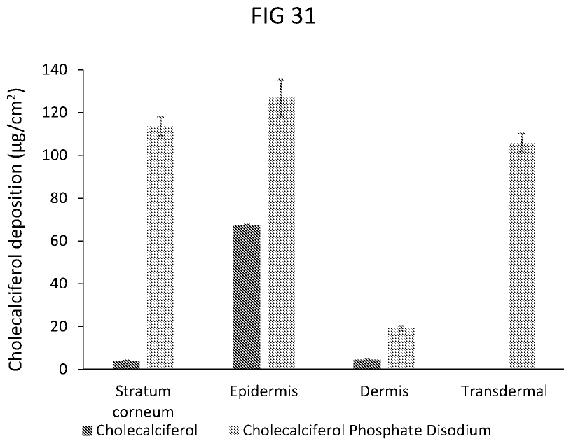

A skin permeation study was conducted using 2.5 mg/mL cholecalciferol (100,000 IU) and 2.5 mg cholecalciferol phosphate disodium (81,967 IU) in pure PG as the donor solutions to determine the effect of the addition of the phosphate on the cholecalciferol permeation process. After 24 h, no cholecalciferol was detected in the receiver fluid (FIG. 31). The deposition study showed that the total cholecalciferol deposited in the skin was 152 μg (6,080 IU) with the majority being in the epidermis layer (135 μg). A total of 210 μg (6885 IU) cholecalciferol phosphate penetrated through the skin into the receiver fluid over 24 h. The deposition study showed that the total cholecalciferol phosphate deposited in the skin was 519 μg (17,016 IU) with the lowest being in the dermis layer (40 μg) and the rest almost equally split between SC (228 μg) and epidermis (250 μg). As the deposition of cholecalciferol phosphate was significantly higher than the cholecalciferol in each layer of the skin (p<0.05, ANOVA) and there was only transdermal permeation when the phosphate was present this demonstrated that the phosphorylated form of vitamin D had superior skin absorption.

### 3.5.3. Part 3A: Patch Development

The patch development process was performed using a systematic process. Initially a common solvent for both drug and polymers was identified. This was followed by optimizing drug loading, adhesive properties, adhesive layer thickness, and inclusion/percentage of permeation enhancers.

3.5.3.1 Cholecalciferol Phosphate (disodium and acid) Patch Development

### Cholecalciferol Phosphate Disodium Adhesive Suitability

DURO-TAK 87-9301 (Table 48) did not show compatibility, i.e., did not dissolve in a common solvent, with cholecalciferol phosphate disodium. Other adhesives (Oppanol B12 and Oppanol N100) (Table 49) also did not show acceptable miscibility. Therefore, these adhesives were unsuitable for patch production.

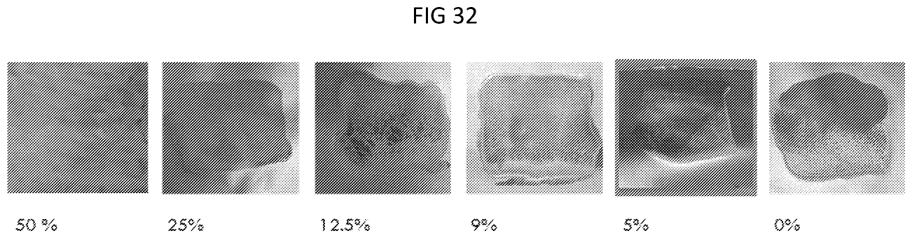

A polar grade of DURO-TAK (87-2516), with the presence of —OH group, was compatible with the cholecalciferol phosphate disodium (Table 50). A 3, 5, and 10% w/w loading of cholecalciferol phosphate disodium generated clear formulations (Table 50). When mixed with all the constituents of the formulation this resulted in solutions with an active concentration of 0.1, 0.3, 0.6 and 1.25 mg/mL. The presence of drug crystals was checked using a light microscope after the formulations were cast into patches and dried (FIG. 32). The 0.6 mg/mL active loading was the highest concentration that did not have any crystallization and generated patch F1. However, because of the poor solubility of the disodium cholecalciferol phosphate in the patch formulations subsequent experiments attempted to use the acid form of cholecalciferol phosphate.

TABLE 48
DURO-TAK 87-9301 suitability screen.

| Cholecal- | Adhesive | | | | | | | |
|---|---|---|---|---|---|---|---|---|
| ciferol | DURO- | | Solvents | | | | | |
| Phosphate | TAK | | | Ethyl | | | | |
| Disodium | 87-9301 | | Methanol | acetate | | PG | | Solubility |
| — | 0.5 | ml | — | 1 | ml | 1 | mL | Opaque |
| — | 1 | ml | — | — | | 40-360 | μL | Clear |
| — | 0.5 | mL | 0.5 mL | — | | — | | Separated |
| — | 0.5 | mL | 0.5 mL | 0.5 | mL | — | | Clear |
| 4 mg | — | | 4 mL | — | | 40 | μL | Clear |
| 4 mg | 0.2 | mL | — | 1-4 | mL | 60 | μL | Opaque |

TABLE 49
Oppanol B12 and Oppanol N100 suitability screen.

| | Adhesive | | | | | |
|---|---|---|---|---|---|---|
| Cholecal- | (Oppanol | | | | | |
| ciferol | B12 and | | Solvents | | | |
| Phosphate | Oppanol | | | | Povidone | |
| Disodium | N100 | | Hexane | PEG4000 | K90 | Solubility |
| — | 1.2 g | B12 | 12.2 | — | — | Soluble |
| | 0.6 g | N100 | mL | | | |
| 4 mg | 1.2 g | B12 | 58% | 20% | — | Not |
| | 0.6 g | N100 | | | | soluble |
| 4 mg | 12% | B12 | 58% | — | 20% | Not |
| | 6% | N100 | | | | soluble |

TABLE 50
DURO-TAK 87-2516 suitability screen.

| | Adhesive | | | | | | | |
|---|---|---|---|---|---|---|---|---|
| Cholecalciferol | (DURO- | | Solvents | | | | | |
| Phosphate | TAK 87- | | | Ethyl | | | | |
| Disodium | 2516) | | Methanol | acetate | | PG | Water | Solubility |
| — | 0.5 | mL | — | 0.5 | mL | — | — | Clear |
| — | 0.5 | mL | — | 0.5 | mL | 50 uL | — | Clear |
| — | 0.5 | mL | 0.5 mL | — | | — | — | Cloudy |
| — | 0.5 | mL | 0.5 mL | 0.5 | mL | — | — | Clear |
| 3% | 200 | μL | 0.9 mL | 2 | mL | 40 μL | 100 μL | Clear |
| 5% | 180 | μL | 1.4 mL | 2.5 | mL | 4 μL | 150 μL | Clear |
| 10% w/v | 100 | μL | 0.5 mL | 0.5-1.5 | mL | 2 μL | — | Not clear |
| 10% w/v | 93.2 | μL | 1.4 mL | 2.75 | mL | 2 μL | 150 μL | Clear |

### Colecalciferol Phosphate Acid Adhesive Suitability

The cholecalciferol phosphate acid, unlike the disodium salt, showed compatibility with several adhesives and organic solvents including Oppanol B12, DURO-TAK 87-2516, 87-9301 and 87-2052. Table 51 summarises the miscible combinations of the active and the patch excipients. For each solution of active and adhesive, the percentage of active in the formulation was optimised by screening different concentrations of active and identifying the concentration where no drug precipitation was not noted following the casting the drug:adhesive solution on a patch backing sheet. FIG. 18 shows the optimisation of the formulation F2 as an example of this process.

The optimised formulation for Oppanol-based (F2-F4) and DurotTak based (F5-F9) formulations are summarised in Tables 40 and 41 in the method section

TABLE 51
Adhesive suitability scan for cholecalciferol phosphate acid.

| | Amount | | Cholecalciferol | | | | | | | |
|---|---|---|---|---|---|---|---|---|---|---|
| | of | | phosphate | | | Dodecyl | | Ethyl | | |
| Adhesive | adhesive | | acid | Hexane | | amine | Methanol | acetate | | Solubility |
| Oppanol | 1.2 | g | 65 mg | 15.9 | mL | | | | | Clear |
| B12 | 2.3 | g | 130 mg | 32 | mL | | | | | Clear |
| | 1.2 | g | 65 mg | 15.9 | mL | 65 mg | | | | Clear |
| Duro- | 100 | mg | 10 mg | | | 10 mg | 1.5 mL | 1 | mL | Clear |
| TAK 87- | | | | | | | 1 mL | 1.25 | mL | Clear |
| 2516 | | | | | | | 0.5 mL | 0.5 | mL | Clear |
| DURO- | 100 | mg | 5 mg | | | 5 mg | 0.5 mL | 0.25 | mL | Clear |
| TAK 87- | | | | | | | | | | |
| 9301 | | | | | | | | | | |
| DURO- | 0.268 | mL | 10 mg | 0.5 | mL | | | 0.4 | mL | Clear |
| TAK 87- | | | | | | | | | | |
| 2052 | | | | | | | | | | |

### 3.5.3.2 Drug Release Testing Method Development

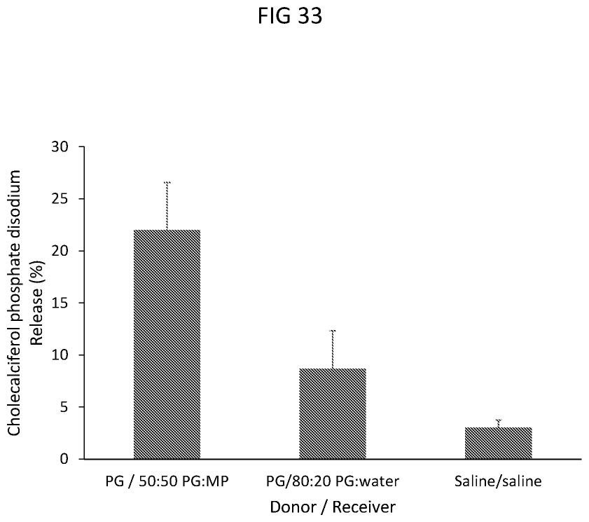

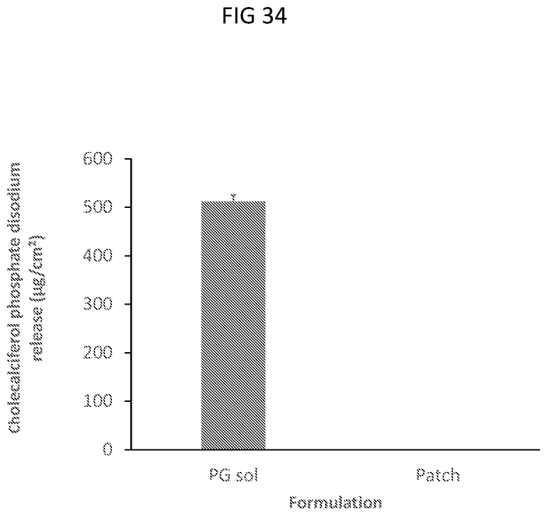

The effect of donor and receiver solvents on the diffusion of cholecalciferol phosphate disodium through regenerated cellulose membrane was investigated (FIG. 33). When employing a PG donor solution, the 50:50 PG:MP (90:10 methanol:water pH 3.2) produced the most extensive release, it delivered 22.0±4.6%. The lowest percentage release was observed using a saline donor with a saline receiver which provided 3.0±0.7% release. The release from all the systems were statistically different (p<0.05, ANOVA). Each donor/receiver combination was suitable to test active release, but a PG donor with 50:50 PG:MP which allowed 423±88 μg/cm² or 13,868±2885 IU to release was the most suitable when the active release from the applied formulation low to enable the best assay sensitivity.

3.5.3.3 Cholecalciferol phosphate disodium patch release testing

The active release from the DURO-TAK 387-2516 cholecalciferol phosphate disodium (F1) patch (Table 39) was examined using a nylon membrane and compared to a control solution of cholecalciferol phosphate disodium in PG (patch vs sol, FIG. 20). The patch had no detectable active release and was inferior to cholecalciferol phosphate disodium control solution, which released 512±17 μg/cm² over 24 h (17% of the applied active, PG sol). The poor release of cholecalciferol phosphate from the patch was presumably due to the polarity of the salt form of the active which favoured interactions with the adhesive rather than releasing into the receiver fluid. As a consequence, the subsequent experiments focused on the acid form of the active in patch designs F2-F9.

### 3.5.3.4 Cholecalciferol Phosphate Acid Patch Release Studies

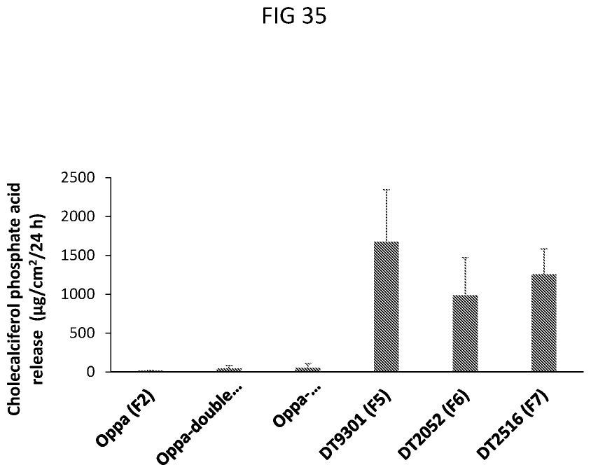

The cholecalciferol phosphate acid release using regenerate cellulose membrane in Franz diffusion cells from 6 different patches (Table 40) was investigated. The acrylate copolymer-based patches (F5-F7) were generally superior in terms of release compared to polyisobutene based patches (F2-F4). The patch that was made with DURO-TAK 87-9301 had the highest release, 1678±669 μg/cm², which corresponded to 55,016±21,934 IU, but there was no significant difference between the 3 different DURO-TAK patches (p>0.05, FIG. 35). It was decided to focus the subsequent development on DURO-TAK 87-2052 (F6) due its good release and the absence of the solubility enhancer (dodecyl amine).

### 3.5.3.5 Cholecalciferol Phosphate Acid Patch Permeation and Deposition

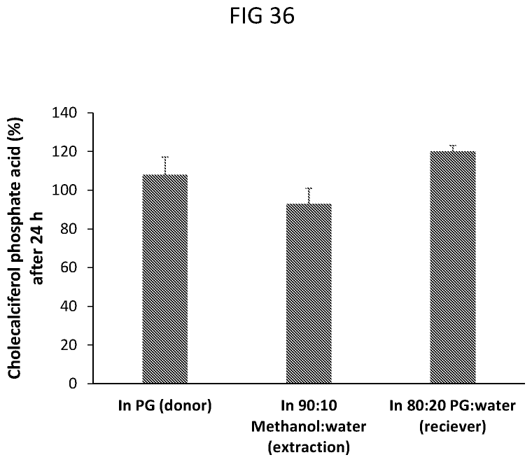

Cholecalciferol phosphate acid showed an appropriate stability in the donor, extraction and receiver solvent (FIG. 36) over 24 hours. A total of 80±9% was recovered from spiked dermatomed porcine skin (data not shown). Due to these recovery percentages then no correction was applied to the subsequent experiments.

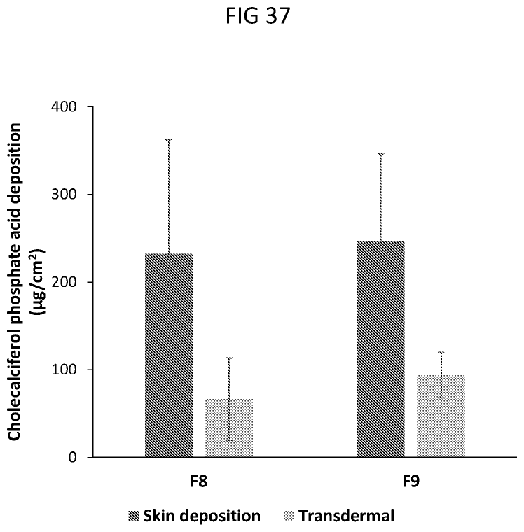

The permeation of cholecalciferol phosphate acid from the two test patches (F8 and F9) (Table 41) was tested using porcine skin (FIG. 37). Formulation F8 delivered 67±47 μg/cm²/24 h (4403±3133 IU) across the skin and formulation F9 delivered 94±26 μg/cm²/24 h (6204±1722 IU) across the skin. The deposition within the sin was 232±130 μg/cm²/24 h (7677±56 IU) and 247±100 μg/cm²/24 h (8168±40 IU) for F8 and F9 respectively. There was no statistically significant differences between the two patches in terms of deposition or transdermal permeation (p>0.05, ANOVA).

TABLE 52
Permeation of cholecalciferol phosphate acid from drug in adhesive
patch (F8 and F9).

| | Concentration (μg/cm²/24 h) | | IU/cm²/24 h | |
|---|---|---|---|---|
| | F8 | F9 | F8 | F9 |
| Skin | <LOD | <LOD | <LOD | <LOD |
| surface | | | | |
| Skin | 232.0 ± 129.9 | 246.8 ± 99.6 | 7606.6 ± 4259.0 | 8091.8 ± 3265.6 |
| deposition | | | | |
| Receiver | 66.54 ± 47.35 | 93.77 ± 26.04 | 2199.7 ± 1565.3 | 3100.8 ± 859.5 |

Considering the Sponsor would like to apply a 1 inch×1 inch patch (6.45 cm²) for 12 h the data in Table 52 for F9 would correspond to 26,093 IU in the skin and 10,000 IU through the skin from a patch containing 66, 788 IU of the active.

TABLE 53
Example of vitamin D Phosphate Patch

| | Required | | Formulation Solid |
|---|---|---|---|
| Materials | Quantity | | Content (%) |
| Vitamin D Phosphate | 480 | mg | 7.9 |
| Transcutol ® CG | 720 | μL | 11.9 |
| Tween 85 | 1200 | μL | 19.8 |
| Methanol | 18 | mL | n/a |
| Ethyl Acetate | 12 | mL | n/a |
| Durotak (387-2051) | 6834 | μL | 57.4 |
| Propyl Gallate | 182.1 | mg | 3 |

For 30 mL formulation=two 30×25×0.018 cm sheets with overage.

The solution was applied on polyester or paper-based backing (80 μm thickness) using a micro applicator set at a height of 350 μm and placed in position on the applicator bed set at 40° C. Following drying of formulation at 70° C. for 30 min or at room temperature overnight, the backing was applied.

TABLE 54
Second Example of vitamin D phosphate patch

| | Required | | Formulation Solid |
|---|---|---|---|
| Materials | Quantity | | Content (%) |
| Vitamin D Phosphate | 480 | mg | 7.9 |
| Transcutol ® CG | 720 | μL | 11.9 |
| Tween 85 | 1200 | μL | 19.8 |
| Methanol | 18 | mL | n/a |
| Ethyl Acetate | 12 | mL | n/a |
| Durotak (387-2051) | 6834 | μL | 57.4 |
| Butylated Hydroxyanisole | 182.1 | mg | 3 |

The patches were produced as above.

### 3.6 Conclusions

Cholecalciferol phosphate (vitamin D phosphate) was synthesised in both the disodium salt and acid forms. Structural analysis experiments evidenced the two-step synthetic procedure was successful in the addition of the phosphate, but the purity of the final product, which ranged from 80-97% suggests that further process optimisation is needed prior to translation into synthesis at commercial scale. When the cholecalciferol phosphate was compared to cholecalciferol in skin permeation studies using a propylene glycol solution, the phosphorylated version of the vitamin delivered 5 times more vitamin D into the skin compared to cholecalciferol (regular non-phosphorylated form) with 210 μg (6,885 IU) delivered through the skin compared to 0 for cholecalciferol. It was also noted that that both cholecalciferol and cholecalciferol phosphate showed an indication of chemical instability and it is suggested that this should be addressed by suitable formulation approaches prior to commercial development. Compatibility studies with patch adhesives demonstrated poor solubility of the cholecalciferol phosphate sodium salt in commonly used adhesives whilst the cholecalciferol phosphate (acid form) dissolved in hexane was readily compatible. DURO-TAK 87-2052 showed good release of cholecalciferol phosphate and when combined with Transcutol® it delivered 3,100.8±859.5 IU of vitamin D per cm² of skin surface. Using a 1 inch square patch the studies predicted that 10,000 IU would pass through the skin which may provide the possibility of once weekly dosing as the daily target dose is 400-1000 IU.

The patch developed in this project exceeds the target transdermal dose by 10-fold, it uses commercially available excipients and can be produced using a traditional manufacture process, thus represents a very strong commercial opportunity.

### REFERENCES

All references, including publications, patent applications, and patents, cited herein are hereby incorporated by reference to the same extent as if each reference were individually and specifically indicated to be incorporated by reference and were set forth in its entirety herein.
1. Ågren M S. Percutaneous Absorption of Zinc from Zinc Oxide Applied Topically to Intact Skin in Man. Dermatologica 1990; 180:36-39.
2. Chang, H., Kim, B. H., Jeong, H. Y., Moon, J. H., Park, M., Shin, K., Chae, S. I., Lee, J., Kang, T., Choi, B. K. and Yang, J., 2019. Molecular-level understanding of continuous growth from iron-oxo clusters to iron oxide nanoparticles. Journal of the American Chemical Society, 141(17), pp. 7037-7045.
3. Stookey, L. L., 1970. Ferrozine—a new spectrophotometric reagent for iron. Analytical chemistry, 42(7), pp. 779-781.
4. Gibbs, C. R., 1976. Characterization and application of ferrozine iron reagent as a ferrous iron indicator. Analytical Chemistry, 48(8), pp. 1197-1201.
5. Carter, P., 1971. Spectrophotometric determination of serum iron at the submicrogram level with a new reagent (ferrozine). Analytical biochemistry, 40(2), pp. 450-458.
6. FDA (CDER), 1997, Guidance for industry—SUPAC-SS Non-sterile Semisolid Dosage Form, Scale-up and post approval changes: Chemistry, manufacturing and controls; in vitro release testing and in vivo bioequivalence documentation]
7. Jeitner, T. M., 2014. Optimized ferrozine-based assay for dissolved iron. Analytical biochemistry, 454, pp. 36-37.
8. Mohammed, E., Mohammed, T. and Mohammed, A., 2017. Optimization of an acid digestion procedure for the determination of Hg, As, Sb, Pb and Cd in fish muscle tissue. MethodsX, 4, pp. 513-523.

1. A patch for transdermal delivery of a compound, comprising:
a backing layer;
an active agent layer comprising at least one compound; and
a permeable-membrane release layer configured to transfer the at least one compound to a skin surface.

2. The patch of claim 1, wherein the compound is selected from zinc, iron, vitamin D, copper, selenium, magnesium, or any combinations thereof.

3. The patch of claim 2, wherein vitamin D comprises at least one of cholecalciferol or cholecalciferol phosphate.

4. The patch of claim 1, wherein the at least one compound is a salt comprising a nutrient and at least one counter ion.

5. The patch of claim 4, wherein the counter ion is selected from ethyl maltol, maltol, kojic acid, propionate, hexanoate, oleate, chloride, phosphate, or any combination thereof.

6. The patch of claim 1, wherein the active agent layer further comprises a solvent.

7. The patch of claim 6, wherein the active agent layer further comprises a solubility enhancer.

8. The patch of claim 7, wherein the solubility enhancer is Tween-85.

9. The patch of claim 6, wherein the solvent is selected from propylene glycol, methanol, ethyl acetate, ethanol, hexane, polyethylene glycol, diethylene glycol monoethyl ether, glycerol, triacetin, miglyol, liquid paraffin, sodium laureth sulfate, Tween-85, Span 20, Kolliphore®, Natrsol M250, dimethicone, or any combination thereof.

10. (canceled)

11. The patch of claim 6, wherein the at least one compound and the solvent form a supersaturated solution.

12. The patch of claim 6, wherein the active agent layer is iron maltol, the solvent is polyethylene glycol, and the permeable-membrane release layer is nylon.

13. The patch of claim 12, wherein the patch delivers about 27 μg/cm²/24 hours of iron into the skin.

14. The patch of claim 6, wherein the active agent layer comprises zinc ethyl maltol and PEG400:PEG 1000 (8% w/w:2% w/w) in a supersaturated solution, and the permeable-membrane release layer is nylon.

15. The patch of claim 14, wherein the patch delivers about 55.3 μg/cm²/24 hours of zinc to the skin.

16.-20. (canceled)

21. The patch of claim 1, wherein the active agent layer comprises cholecalciferol phosphate and acrylate adhesive.

22.-23. (canceled)

24. The patch of claim 1, further comprises one or more additives, wherein the one or more additives is a penetration enhancer; and wherein the penetration enhancer is polyethylene glycol.

25. The patch of claim 1, further comprising one or more additives; and wherein the one or more additives is an antioxidant is selected from vitamin E, vitamin E derivatives, butylated hydroxytoluene (BHT), butylated hydroxyanisole, (BHA), L-cysteine, propyl gallate, or any combinations thereof.

26. A method of delivering a compound to a subject in need, comprising:
applying a patch for transdermal delivery of a compound, comprising:
an outer backing layer;
an intermediate active agent layer comprising at least one compound; and
a permeable-membrane release layer configured to transfer the at least one compound to a skin surface.

27. A method of synthesizing a nutrient-counter ion salt for transdermal delivery, comprising:
providing at least one nutrient;
providing at least one counter ion;
solubilizing the at least one nutrient and the at least one counter ion in at least one solvent to produce a solution;
optionally adjusting a pH of the solution; and
removing the solvent from the solution to produce a nutrient-counter ion salt.

28. The method of claim 27, wherein adjusting a pH of the solution further comprises:
adjusting the pH of the solution to between 1.0 and 8.0.

29.-52. (canceled)
